(19) United States
(12) Patent Application Publication
(10) Pub. No.: US 2025/0019373 A1
(43) Pub. Date: Jan. 16, 2025

(54) ARYL OR HETEROARYL DERIVATIVE, AND PHARMACEUTICAL COMPOSITION COMPRISING SAME AS ACTIVE INGREDIENT FOR PREVENTION OR TREATMENT OF KINASE-RELATED DISEASES

(71) Applicant: VORONOI INC., Incheon (KR)

(72) Inventors: Seohyun JO, Incheon (KR); Dahoon MA, Incheon (KR); Yeseul PARK, Incheon (KR); Gyuseok SIM, Incheon (KR); Jungbeom SON, Incheon (KR); Namdoo KIM, Incheon (KR); Sunghwan KIM, Incheon (KR)

(21) Appl. No.: 18/702,203

(22) PCT Filed: Oct. 21, 2022
(86) PCT No.: PCT/KR2022/016188

§ 371 Date: Apr. 17, 2024

(30) Foreign Application Priority Data
Oct. 22, 2021 (KR) 10-2021-0141877
Feb. 4, 2022 (KR) 10-2022-0014856

(51) Int. Cl. C07D 413/14; A61K 31/422; A61K 31/423; A61K 31/428; A61K 31/437; A61K 31/4439; A61K 31/444; A61K 31/5025; A61K 31/519; A61K 31/5355; A61K 31/5377; C07D 413/12; C07D 417/12; C07D 471/04; C07D 487/04; C07D 495/04; C07D 513/04

(52) U.S. Cl. CPC C07D 413/14; A61K 31/422; A61K 31/423; A61K 31/428; A61K 31/437; A61K 31/4439; A61K 31/444; A61K 31/5025; A61K 31/519; A61K 31/5355; A61K 31/5377; C07D 413/12; C07D 417/12; C07D 471/04; C07D 487/04; C07D 495/04; C07D 513/04

(57) ABSTRACT

The present invention relates to an aryl or heteroaryl derivative and a pharmaceutical composition for the treatment of kinase-related diseases comprising same as an active ingredient. An aryl or heteroaryl derivative provided according to an aspect of the present invention exhibits excellent inhibitory activity against kinases, especially receptor-interacting serine/threonine-protein kinase 1 (RIPK1), and thus can be used as a therapeutic agent for RIPK1-related diseases.

### TECHNICAL FIELD

The present invention relates to an aryl or heteroaryl derivative, and a pharmaceutical composition for preventing or treating a kinase-related disease, which includes the aryl or heteroaryl derivative as an active ingredient.

### BACKGROUND ART

Protein kinases are enzymes that catalyze a phosphorylation reaction that transfers a γ-phosphate group of ATP to the hydroxy groups of tyrosine, serine, and threonine in proteins, and are responsible for cell metabolism, gene expression, cell growth, differentiation, and cell division and play an important role in cell signaling (Non-Patent Document 1, Thomas A. Hamilton, in Encyclopedia of Immunology (Second Edition), 1998, 2028-2033). Protein kinases are classified into tyrosine protein kinases and serine/threonine kinases and more than 90 types are tyrosine kinases.

Protein kinases are molecular switches that must actively regulate the transition between active and inactive states in cells. When the transition between the active and inactive states is abnormally regulated, intracellular signaling is excessively activated, leading to uncontrolled cell division and proliferation. In addition, the abnormal activation of a protein kinase due to genetic mutation, amplification, and overexpression is associated with the development and progression of various tumors and plays a critical role in the development of various diseases such as inflammatory diseases, degenerative brain diseases, autoimmune diseases, and cancer. Examples of the related kinases include ABL1, ABL2, BRAF, CDK11, CDK8, CDKL2, CIT, CSF1R, DDR1, DDR2, FLT3, KIT, LOK, LTK, MUSK, PAK3, PDGFRA, PDGFRB, RAF1, and RIPK1. Receptor-interacting serine/threonine-protein kinase 1 (RIPK1), which is one example of a protein kinase, is a multifunctional signal transducer that is involved in mediating NF-κB activation, apoptosis, and/or necroptosis.

Particularly, RIPK1 activity is known to be critically involved in mediating necroptosis, which is a caspase-independent pathway of necrotic cell death (Non-Patent Document 2, Holler et al., Nat Immunol 2000; 1:489-495; Non-Patent Document 3, Degterev et al., Nat Chem Biol 2008; 4:313-321).

Accordingly, if RIPK1 activity can be effectively inhibited, cell protection is possible under conditions for inducing cell death caused by tumor necrosis factor-alpha (TNF-α), thereby blocking necroptosis.

It has been reported that RIPK1-mediated necroptosis is associated with various diseases such as inflammatory diseases, degenerative brain diseases, autoimmune diseases, and cancer.

First, since it has been confirmed that RIP3 knockout mice designed to completely block RIPK1-mediated programmed necrosis have protective effects in inflammatory bowel diseases (including ulcerative colitis and Crohn's disease) (Non-Patent Document 4, (2011) Nature 477, 330-334), psoriasis (Non-Patent Document 5, (2011) Immunity 35, 572-582), retinal detachment-induced photoreceptor necrosis (Non-Patent Document 6, (2010) PNAS 107, 21695-21700), retinitis pigmentosa (Non-Patent Document 7, (2012) Proc. Natl. Acad. Sci., 109:36, 14598-14603), cerulein-induced acute pancreatitis (Non-Patent Document 8, (2009) Cell 137, 1100-1111), and sepsis/systemic inflammatory response syndrome (SIRS) (Non-Patent Document 9, (2011) Immunity 35, 908-918), if a specific compound can block RIPK1-mediated necroptosis, it is possible to develop the corresponding compound as a treatment for an inflammatory disease.

In addition, since it is known that RIPK1 mediates microglial responses in Alzheimer's disease (Non-Patent Document 10, PNAS Oct. 10, 2017. 114 (41) E8788-E8797), if a specific compound can target RIPK1 to effectively inhibit its activity, it is possible to develop the corresponding compound as a treatment for degenerative brain diseases (i.e., degenerative neurological diseases) such as Alzheimer's disease, Down syndrome, Parkinson's disease, Lou Gehrig's disease, dementia, Huntington's disease, multiple sclerosis, amyotrophic lateral sclerosis, palsy, stroke, and mild cognitive impairment.

Further, it is known that RIPK1 regulates the production of tumor necrosis factor-alpha (TNF-α), and TNF-α is a pro-inflammatory cytokine that is involved in mediating cell death and inflammation in various diseases, including rheumatoid arthritis and cancer (Non-Patent Document 11, Cell Death and Disease (2012) 3, e320). Therefore, if a specific compound can target RIPK1 to effectively inhibit activity, it is possible to develop the corresponding compound as a treatment for rheumatoid arthritis, polymyalgia rheumatica, ankylosing spondylitis, an autoimmune disease such as motor neurone disease, and cancer.

In addition, it has been disclosed that RIPK1 induces macrophage-mediated adaptive immune tolerance in pancreatic cancer (Non-Patent Document 12, Cancer Cell 34, 757-774, Nov. 12, 2018). More specifically, it has been shown that RIPK1 inhibition in TAMs results in cytotoxic T cell activation and T helper cell differentiation toward a mixed Th1/Th17 phenotype, resulting in tumor immunity in mice and organotypic models of human PDA.

Therefore, since targeting RIPK1 acts synergistically with PD-1 and inducible co-stimulator-based immunotherapies, it can be seen that RIPK1 is a checkpoint kinase that governs tumor immunity. That is, since it is possible to develop a compound that can effectively inhibit the activity of a protein kinase, including RIPK1, as a treatment for various diseases such as inflammatory diseases, degenerative brain diseases, autoimmune diseases, and cancer, there is a need for the development of a protein kinase inhibitor with a novel structure.

### DISCLOSURE

### Technical Problem

The present invention is directed to providing an aryl or heteroaryl derivative with a novel structure, an isomer thereof, a solvate thereof, a hydrate thereof, or a pharmaceutically acceptable salt thereof, which exhibits excellent inhibitory activity against various kinases and thus has a therapeutic effect on kinase-related diseases.

### Technical Solution

To achieve the above purpose, one aspect of the present invention provides a compound represented by Chemical Formula 1, an isomer thereof, a solvate thereof, a hydrate thereof, or a pharmaceutically acceptable salt thereof:

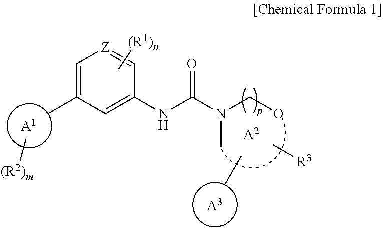

In Chemical Formula 1,
Z is N or CH,
ring A¹ is 5- to 12-membered heteroaryl,
ring A² is 5- to 10-membered heterocycloalkyl, 5- to 10-membered heterocycloalkyl-aryl bicyclic ring, or 5- to 10-membered heterocycloalkyl-heteroaryl bicyclic ring, wherein p is an integer from 0 to 2,
ring A³ is 4- to 12-membered aryl or 4- to 12-membered heteroaryl, and is unsubstituted or substituted with one or more substituents R⁴ selected from halogen, cyano, C₁₋₈ alkoxy, C₁₋₈ alkyl, C₁₋₈ haloalkyl, and C₁₋₈ alkylamino,
R¹ is hydrogen, halogen, C₁₋₈ alkyl, C₁₋₈ alkoxy, cyano, C₃₋₈ cycloalkyl, or C₃₋₈ heterocycloalkyl, and n is an integer from 1 to 3,
R² is halogen, C₁₋₈ alkyl, C₁₋₈ alkoxy, amine, cyano, C₁₋₈ haloalkyl, or C₂₋₈ alkynyl that is unsubstituted or substituted with a hydroxyl group,

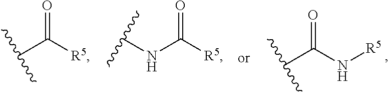

and m is an integer from 0 to 4,
R⁵ is C₁-8 alkyl, —C₁₋₈ alkyl-C₁₋₈ alkoxy, —C₁₋₈ alkyl-hydroxy, or C₃₋₈ cycloalkyl, where the C₃₋₈ cycloalkyl of R⁵ is unsubstituted or substituted with C₁₋₈ alkylamino or halogen, and
R³ is hydrogen or C₁₋₈ alkyl.

Another aspect of the present invention provides a method of preparing a compound of Chemical Formula 1, which includes preparing a compound of Chemical Formula 3 from a compound of Chemical Formula 2 (Step 1); and
preparing a compound of Chemical Formula 1 from the compound of Chemical Formula 3 (Step 2).

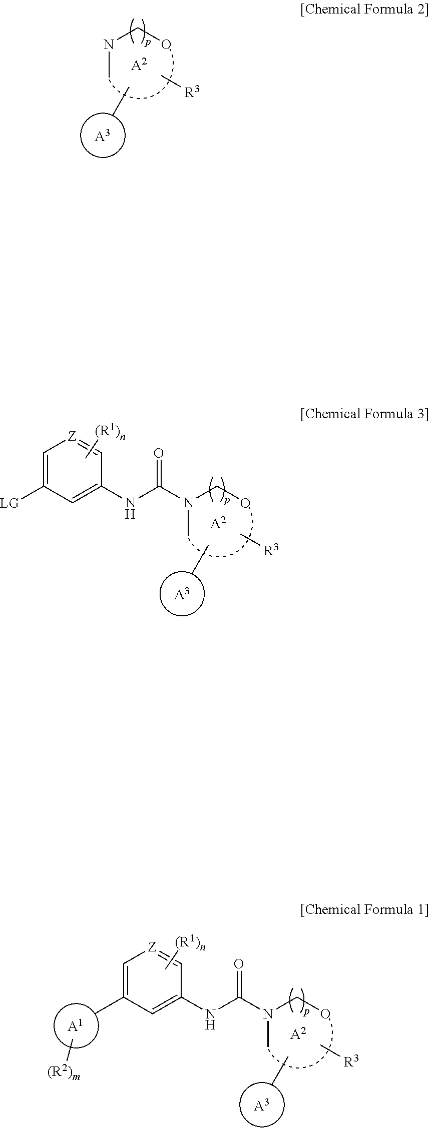

In the above Formulas, LG is a leaving group, and Z, rings A¹ to A³, R¹ to R³, m, n, and p may be the same as defined in this specification.

Still another aspect of the present invention provides a pharmaceutical composition for preventing or treating one or more of kinase-related diseases, which includes the above compound, an isomer thereof, a solvate thereof, a hydrate thereof, or a pharmaceutically acceptable salt thereof as an active ingredient.

### Advantageous Effects

A compound provided in one aspect of the present invention, an isomer thereof, a solvate thereof, a hydrate thereof, or a pharmaceutically acceptable salt thereof exhibits excellent inhibitory activity against kinases, especially RIPK1, and thus has an excellent inhibitory effect on cell necrosis. Therefore, it can be effectively used as a treatment for the kinase-related diseases.

### [Modes of the Invention]

Hereinafter, the present invention will be described in detail.

Meanwhile, embodiments of the present invention may be modified in various different forms, and the scope of the present invention is not limited to embodiments to be described below. In addition, the embodiments of the present invention are provided to more completely explain the present invention to those of ordinary skill in the art. Further, when a certain part “includes” a certain component, it means that, unless specifically stated otherwise, another component may be further included, rather than excluded.

Hereinafter, the present invention will be described in detail with reference to the following embodiments.

Embodiments of the present invention may be modified in a variety of different forms, and the scope of the present invention is not limited to embodiments to be explained below. In addition, the embodiments of the present invention are provided to more completely explain the present invention to those of ordinary skill in the art.

Further, when a certain part “includes” a certain component, it means that, unless particularly stated otherwise, another component may be further included, rather than excluded.

In the structural formulas of this specification, the symbol “−” and “=”, which combine atoms and/or groups, may refer to a single bond and a double bond, respectively. These symbols may be omitted, and may be displayed, if necessary, for example, when specifying a bonding atom or a bonding position.

In this specification, “linking” between atoms may represent not only the case of directly linking atoms but also the case of indirectly linking atoms via another atom and/or group. Here, the other atom and/or group may be, but are not limited to, oxygen, sulfur, a C₁₋₈ alkylamino, or a C₁₋₈ alkylene, and the atom and/or group may be substituted or unsubstituted.

In this specification, “substituted or unsubstituted” may mean that, unless otherwise specified, one or more hydrogen atoms are substituted with another atom or substituent, or not substituted. The substituent may be, but is not limited to, one or more selected from the group consisting of halogen (chloro (Cl), iodo (I), bromo (Br), or fluoro (F)), C₁₋₁₀ alkyl, C₂₋₁₀ alkenyl, C₂₋₁₀ alkynyl, hydroxyl, C₁₋₁₀ alkoxy, amino, nitro, thiol, thioether, imine, cyano, phosphine, carboxy, carbamoyl, acetal, thiocarbonyl, sulfonyl, sulfonamide, ketone, aldehyde, ester, acetyl, amide, oxygen (═O), haloalkyl (e.g., trifluoromethyl), substituted aminoacyl and amino alkyl, monocyclic or fused or unfused polycyclic carbon ring cycloalkyl (e.g., cyclopropyl, cyclobutyl, cyclopentyl, or cyclohexyl), or monocyclic or fused or unfused polycyclic heterocycloalkyl (e.g., pyrrolidinyl, piperidinyl, piperazinyl, morpholinyl, or thiazinyl), cyclic or heterocyclic, monocyclic, or fused or unfused polycyclic aryl (e.g., phenyl, naphthyl, pyrrolyl, indolyl, furanyl, thienyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridizinyl, pyrimidinyl, benzimidazolyl, benzothienyl, or benzofuranyl), amino (primary, secondary or tertiary), aryl, aryloxy, and aryl-alkyl. In addition, each of the exemplified substituents may be unsubstituted or substituted with a substituent selected from the above substituent group.

In this specification, “halogen” may be F, Cl, Br, or I.

In this specification, “alkyl” may refer to, unless otherwise stated, a saturated hydrocarbon of a straight-chain or branched chain noncyclic alkyl; a cyclic alkyl; or one formed by the combination of the both above-mentioned alkyls. In addition, “C₁₋₈ alkyl” may refer to alkyl containing 1 to 8 carbon atoms. The acyclic alkyl may include, for example, but is not limited to, methyl, ethyl, N-propyl, N-butyl, isopropyl, sec-butyl, isobutyl, tert-butyl, isopentyl, or 2-methylpentyl. The cyclic alkyl may include, for example, but is not limited to, cyclopropyl, cyclobutyl, cyclopentyl, or cyclohexyl. The alkyl, which is a combination of acyclic and cyclic alkyl, may include, for example, but is not limited to, methylcyclopropyl, cyclopropylmethyl, ethylcyclopropyl, or cyclopropylethyl.

In this specification, “cycloalkyl” may refer to, particularly, a cyclic alkyl among alkyls, where the alkyl is as defined above.

In this specification, “heterocycloalkyl” may refer to a ring containing 1 to 5 hetero atoms selected from N, O, and S as ring-forming atoms, and may be saturated or partially unsaturated. Unless otherwise specified, a heterocycloalkyl may be monocyclic, or polycyclic such as a spiro ring, a bridged ring, or a fused ring. In addition, “3- to 10-membered heterocycloalkyl” may refer to a heterocycloalkyl that include 3 to 10 ring-forming atoms, including pyrrolidine, piperidine, N-methylpiperidine, imidazolidine, pyrazolidine, imidazolidinone, piperidine, pyrimidine-2,4(1H,3H)-dione, 1,4-dioxane, morpholine, thiomorpholine, piperazine, pyran, pyridone, 3-pyrroline, thiopyran, pyrone, tetrahydrofuran, tetrahydrothiophene, 2-azaspiro[3.3]heptane, isoxazolidine, benzo[d]isoxazolidine, oxazinane, 5-oxa-6-azaspiro[2.4]heptane, (1R,5S)-3-azabicyclo[3.2.1]octane, (1s,4s)-2-azabicyclo[2.2.2]octane, or (1R,4R)-2-oxa-5-azabicyclo[2.2.2]octane, but the present invention is not limited thereto.

In this specification, “bicyclic ring” may refer to two rings that are fused while having one or more atoms in common. Specifically, it may be formed by a common single or double bond (polycyclic bicyclic ring), by sharing three or more arranged atoms (bridged bicyclic ring), or through a common single atom (spiro bicyclic ring). A bicyclic ring may be a saturated, partially unsaturated, unsaturated, or aromatic ring, and may include a hetero atom. Moreover, the ring may also include a ring in which hydrogen has been removed.

In this specification, “alkoxy” may refer to an alkyl ether group, i.e., —(O-alkyl), where the alkyl is as defined above. In addition, “C₁₋₈ alkoxy” may refer to alkoxy containing C₁₋₈ alkyl, i.e., —(O—C₁₋₈ alkyl).

In this specification, “alkylamino” may refer to —(NR′R″), where R′ and R″ may each be independently selected from the group consisting of hydrogen and C₁₋₈ alkyl, and the selected R′ and R″ may each be independently substituted or unsubstituted. In addition, “C₁₋₈ alkylamino” may refer to C₁₋₈ alkyl-containing amino, that is, —N—H(C₁₋₈ alkyl) or —N—(C₁₋₈ alkyl)₂, and may be, but is not limited to, dimethylamino, diethylamino, methylethylamino, methylpropylamino, or ethylpropylamino.

In this specification, “haloalkyl” may refer to —RX (X is one or more halogens (F, Cl, Br, or I)). In other words, “haloalkyl” may be an alkyl form substituted with one or more halogens. For example, “C₁₋₈ haloalkyl” may be, but is not limited to, trifluoromethyl or difluoromethyl.

In this specification, “haloalkoxy” may refer to —(O—RX) (X is one or more halogens (F, Cl, Br, or I)). In other words, “haloalkoxy” may be an alkoxy form substituted with one or more halogens. For example, “C₁₋₈ haloalkoxy” may be, but is not limited to, trifluoromethoxy or difluoromethoxy.

In this specification, “aryl” may refer to an aromatic ring in which one hydrogen is removed from an aromatic hydrocarbon ring, and may be a monocyclic ring or a polycyclic ring. “C₃₋₁₂ aryl” may refer to an aryl including 3 to 12 ring-forming atoms, and may be, for example, but is not limited to, phenyl, benzyl, naphthyl, anthracenyl, phenanthryl, biphenyl, or terphenyl.

In this specification, “heteroaryl” may refer to an aromatic ring containing one or more hetero atoms of N, O, and S as ring-forming atoms, and may be a monocyclic ring or a polycyclic ring. In addition, “C₃₋₁₂ heteroaryl” may refer to heteroaryl including 3 to 12 ring-forming atoms, and may be, for example, but is not limited to, thienyl, thiophenyl, furyl, pyrrolyl, pyrazolyl, imidazolyl, thiazolyl, oxazolyl, isothiazolyl, oxadiazolyl, triazoyl, pyridinyl, bipyridinyl, pyrimidinyl, triazinyl, acridyl, pyridazinyl, pyrazinyl, quinolinyl, or quinazoline.

In this specification, “hydroxy” may refer to —OH.

In this specification, “alkenyl” may refer to, unless otherwise stated, a linear or branched, acyclic or cyclic hydrocarbon with one or more double bonds.

In this specification, “cyano” may refer to —(CN).

In this specification, “alkynyl” may refer to, unless otherwise stated, a linear or branched, acyclic or cyclic hydrocarbon with one or more triple bonds. For example, “C₂₋₄ alkynyl” may refer to alkynyl that has 2 to 4 carbon atoms and one or more triple bonds. Specifically, the alkynyl may include, but is not limited to, ethynyl, propyn-1-yl, propyn-2-yl, or methylbutyn-1-yl.

In this specification, “hydrate” may refer to a compound of the present invention that includes a stoichiometric or non-stoichiometric amount of water bound by a non-covalent intermolecular force, or a salt thereof. A hydrate of the compound represented by Chemical Formula 1 of the present invention may include a stoichiometric or non-stoichiometric amount of water bound by a non-covalent intermolecular force. The hydrate may contain 1 eq or more, and preferably, 1 eq to 5 eq of water. Such a hydrate may be prepared by crystallizing the compound represented by Chemical Formula 1 of the present invention, an isomer thereof, or a pharmaceutically acceptable salt thereof from water or a water-containing solvent.

In this specification, “solvate” may refer to a compound of the present invention, which includes a stoichiometric or non-stoichiometric amount of solvent bound by a non-covalent intermolecular force, or a salt thereof. Exemplary solvents are solvents that are volatile, non-toxic, and/or suitable for administration to humans.

In this specification, “isomer” may refer to a compound of the present invention, which has the same chemical or molecular formula, but is structurally or sterically different, or a salt thereof. These isomers include structural isomers such as tautomers, R or S isomers having an asymmetric carbon center, isomers such as geometric isomers (trans, cis), and optical isomers (enantiomers). All of these isomers and mixtures thereof are also included within the scope of the present invention.

The present invention provides a compound of Chemical Formula 1 below, an isomer thereof, a solvate thereof, a hydrate thereof, or a pharmaceutically acceptable salt thereof:

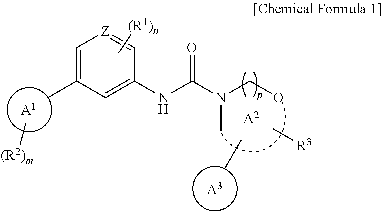

In Chemical Formula 1,
Z is N or CH,
ring A¹ is 5- to 12-membered heteroaryl,
ring A² is 5- to 10-membered heterocycloalkyl, 5- to 10-membered heterocycloalkyl-aryl bicyclic ring, or 5- to 10-membered heterocycloalkyl-heteroaryl bicyclic ring, wherein p is an integer from 0 to 2,
ring A³ is 4- to 12-membered aryl or 4- to 12-membered heteroaryl, and is unsubstituted or substituted with one or more substituents R⁴ selected from halogen, cyano, C₁₋₈ alkoxy, C₁₋₈ alkyl, C₁₋₈ haloalkyl, and C₁₋₈ alkylamino,
R¹ is hydrogen, halogen, C₁₋₈ alkyl, C₁₋₈ alkoxy, cyano, C₃₋₈ cycloalkyl, or C₃₋₈ heterocycloalkyl, and n is an integer from 1 to 3,
R² is halogen, C₁₋₈ alkyl, C₁₋₈ alkoxy, amine, cyano, C₁₋₈ haloalkyl, C₂₋₈ alkynyl which is unsubstituted or substituted with a hydroxyl group,

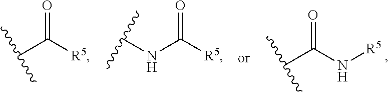

and m is an integer from 0 to 4,
R⁵ is C₁₋₈ alkyl, —C₁₋₈ alkyl-C₁₋₈ alkoxy, —C₁₋₈ alkyl-hydroxy, or C₃₋₈ cycloalkyl, where the C₃₋₈ cycloalkyl of R⁵ is unsubstituted or substituted with C₁₋₈ alkylamino or halogen, and
R³ is hydrogen or C₁₋₈ alkyl.

According to an exemplary embodiment of the present invention, in the compound of Chemical Formula 1,
Z is N or CH,
ring A¹ is 8- to 10-membered heteroaryl that includes one or more hetero atoms selected from the group consisting of N, O, and S,
ring A² is 5- to 8-membered heterocycloalkyl or 8- to 10-membered heterocycloalkyl-aryl bicyclic ring, where p is an integer from 0 to 2,
ring A³ is 5- to 10-membered aryl or 4- to 8-membered heteroaryl including one or more hetero atoms selected from N, O, and S, and is unsubstituted or substituted with one or more substituents R⁴ selected from halogen, cyano, C₁₋₆ alkoxy, C₁₋₆ alkyl, C₁₋₆ haloalkyl, and C₁₋₆ alkylamino,
R¹ is hydrogen, halogen, C₁₋₆ alkyl, C₁₋₆ alkoxy, cyano, or C₃₋₆ cycloalkyl, and n is an integer from 1 to 3,
R² is halogen, C₁₋₆ alkyl, C₁₋₆ alkoxy, amine, cyano, C₁₋₆ haloalkyl, or C₂₋₆ alkynyl that is unsubstituted or substituted with a hydroxyl group,

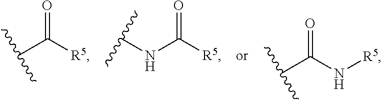

and m is an integer from 0 to 4,
R⁵ is C₁-6 alkyl, —C₁₋₆ alkyl-C₁₋₆ alkoxy, —C₁₋₆ alkyl-hydroxy, or C₃₋₆ cycloalkyl, where the C₃₋₆ cycloalkyl of R⁵ is unsubstituted or substituted with C₁₋₆ alkylamino or halogen, and
R³ is hydrogen or C₁₋₆ alkyl.

According to an exemplary embodiment of the present invention, in the compound of Chemical Formula 1,
ring A¹ is 5- to 12-membered heteroaryl and may include one or more of hetero atoms selected from the group consisting of N, O, and S.

According to an exemplary embodiment of the present invention, ring A¹ is 8- to 10-membered heteroaryl that includes one or more hetero atoms selected from the group consisting of N, O, and S, and more specifically, ring A¹ may include two or more of these hetero atoms.

More specifically, ring A¹ may be imidazo[1,2-b]pyridazine, benzo[d]oxazole, benzo[d][1,2,3]triazole, benzo[d]thiazole, indazole, thiazolo[5,4-b]pyridine, triazolo[1,5-a]pyridine, pyrrolo[2,1-f][1,2,4]triazine, triazolo[4,3-b]pyridazine, imidazo[1,2-a]pyridine, thieno[3,2-d]pyrimidine, pyrazolo[1,5-a]pyridine, thiazolo[4,5-c]pyridine, imidazo[4,5-c]pyridine, or imidazo[4,5-b]pyridine.

According to an exemplary embodiment of the present invention, ring A¹ may be

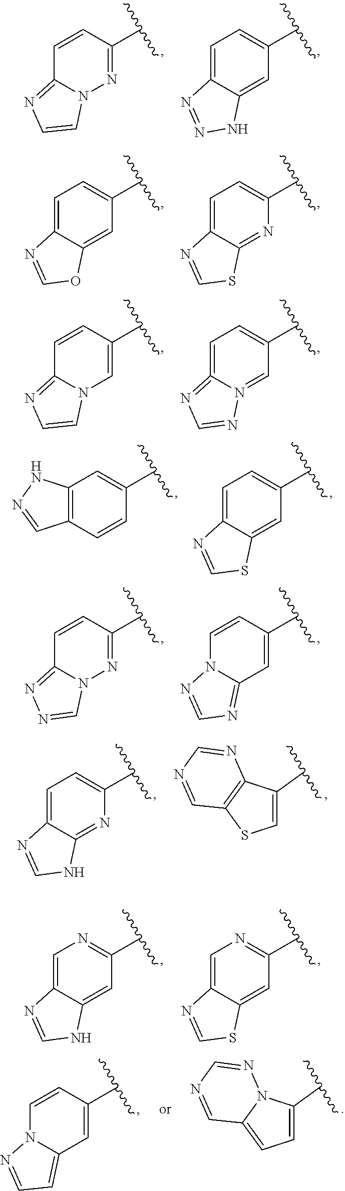

According to an exemplary embodiment of the present invention, in the compound of Chemical Formula 1,
ring A² is 5- to 10-membered heterocycloalkyl, 5- to 10-membered heterocycloalkyl-aryl bicyclic ring, or 5- to 10-membered heterocycloalkyl-heteroaryl bicyclic ring, where p may be an integer from 0 to 2.

Specifically, ring A² may be 5- to 8-membered heterocycloalkyl including hetero atoms of N and O or an 8- to 10-membered heterocycloalkyl-aryl bicyclic ring including hetero atoms of N and O.

More specifically, ring A² may be isoxazolidine, morpholine, benzo[d]isoxazolidine, oxa-azaspiro[2.4]heptane, or oxazinane.

According to an exemplary embodiment of the present invention, in the compound of Chemical Formula 1,
ring A³ is 4- to 12-membered aryl or 4- to 12-membered heteroaryl, and may be unsubstituted or substituted with one or more substituents R⁴ selected from halogen, cyano, C₁₋₈ alkoxy, C₁₋₈ alkyl, C₁₋₈ haloalkyl, and C₁₋₈ alkylamino.

Specifically, ring A³ may be 5- to 10-membered aryl or 4- to 8-membered heteroaryl, more specifically, ring A³ may be 5- to 10-membered aryl or 4- to 8-membered heteroaryl including one or more hetero atoms selected from N, O, and S, and most specifically, ring A³ may be phenyl, pyridinyl, naphthalenyl, thiazolyl, pyrazolyl, or furanyl.

In the present invention, ring A³ may be unsubstituted or substituted with one or more substituents R⁴ selected from halogen, cyano, C₁₋₈ alkoxy, C₁₋₈ alkyl, C₁₋₈ haloalkyl, and C₁₋₈ alkylamino.

Specifically, R⁴ may be selected from halogen, cyano, C₁₋₆ alkoxy, C₁₋₆ alkyl, C₁₋₆ haloalkyl, and C₁₋₆ alkylamino, and more specifically, R⁴ may be selected from halogen, cyano, methoxy, methyl, trifluoromethyl, and dimethylamine.

According to an exemplary embodiment of the present invention, in the compound of Chemical Formula 1,
R¹ is hydrogen, halogen, C₁₋₈ alkyl, C₁₋₈ alkoxy, cyano, C₃₋₈ cycloalkyl, or C₃₋₈ heterocycloalkyl, and n may be an integer from 1 to 3.

More specifically, R¹ may be hydrogen, halogen, C₁₋₆ alkyl, C₁₋₆ alkoxy, cyano, C₃₋₆ cycloalkyl, or C₃₋₆ heterocycloalkyl, and most specifically, R¹ may be hydrogen, halogen, C₁₋₃ alkyl, C₁₋₃ alkoxy, cyano, or C₃₋₆ cycloalkyl.

According to an exemplary embodiment of the present invention, when Z is N, R¹ may be C₁₋₈ alkyl or C₁₋₈ alkoxy.

According to an exemplary embodiment of the present invention, in the compound of Chemical Formula 1,
R² is halogen, C₁₋₈ alkyl, C₁₋₈ alkoxy, amine, cyano, C₁₋₈ haloalkyl, or C₂₋₈ alkynyl that is unsubstituted or substituted with a hydroxyl group,

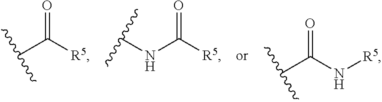

and m is an integer from 0 to 4, and

R⁵ is C₁₋₈ alkyl, —C₁₋₈ alkyl-C₁₋₈ alkoxy, —C₁₋₈ alkyl-hydroxy, or C₃₋₈ cycloalkyl, where the C₃₋₈ cycloalkyl of R⁵ is unsubstituted or substituted with C₁₋₈ alkylamino or halogen.

According to an exemplary embodiment of the present invention, R² may be halogen, C₁₋₆ alkyl, C₁₋₆ alkoxy, amine, cyano, C₁₋₆ haloalkyl, or C₂₋₆ alkynyl that is unsubstituted or substituted with a hydroxyl group,

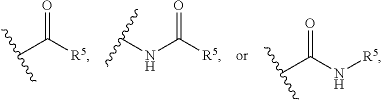

and more specifically, R² may be C₁₋₃ alkyl, amine, cyano, C₁₋₃ haloalkyl, ethynyl, hydroxymethylbutynyl,

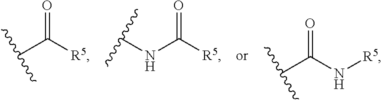

Here, m may be an integer from 0 to 3, and more specifically, an integer from 0 to 2. When m is 0, ring A¹ means an unsubstituted ring.

According to an exemplary embodiment of the present invention, R⁵ is C₁-6 alkyl, —C₁₋₆ alkyl-C₁₋₆ alkoxy, —C₁₋₆ alkyl-hydroxy, or C₃₋₆ cycloalkyl, where the C₃₋₆ cycloalkyl of R⁵ may be unsubstituted or substituted with C₁₋₆ alkylamino or halogen.

According to an exemplary embodiment of the present invention,
when R² is

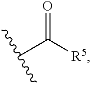

R⁵ is C₃₋₈ cycloalkyl that is unsubstituted or substituted with halogen,
when R² is

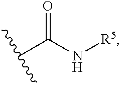

R⁵ is C₁₋₈ alkyl or C₃₋₈ cycloalkyl that is unsubstituted or substituted with C₁₋₈ alkylamino,
when R² is

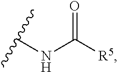

R⁵ may be C₃₋₈ cycloalkyl that is unsubstituted or substituted with C₁₋₈ alkyl, —C₁₋₈ alkyl-C₁₋₈ alkoxy, —C₁₋₈ alkyl-hydroxy, or halogen.

According to an exemplary embodiment of the present invention, in the compound of Chemical Formula 1,
R³ may be hydrogen or C₁₋₈ alkyl, and specifically, R³ may be hydrogen or C₁₋₃ alkyl.

Examples of the compound of Chemical Formula 1 according to the present invention may include Compounds 1 to 158 listed in [Table 2] in the following examples, or free bases (when shown as pharmaceutically acceptable salts in Table 2), isomers thereof, hydrates thereof, solvates thereof, or pharmaceutically acceptable salts thereof.

The compound represented by Chemical Formula 1 of the present invention may be used in the form of a pharmaceutically acceptable salt thereof. Particularly, the pharmaceutically acceptable salt may be an acid addition salt formed by a free acid. Here, acid addition salts may be obtained from inorganic acids such as hydrochloric acid, nitric acid, phosphoric acid, sulfuric acid, hydrobromic acid, hydroiodic acid, nitrous acid, and phosphorous acid; non-toxic organic acids such as aliphatic mono- and di-carboxylates, phenyl-substituted alkanoates, hydroxy alkanoates, and alkanedioates, aromatic acids, and aliphatic and aromatic sulfonic acids; and organic acids such as trifluoroacetic acid, acetate, benzoic acid, citric acid, lactic acid, maleic acid, gluconic acid, methanesulfonic acid, 4-toluenesulfonic acid, tartaric acid, and fumaric acid. The types of such pharmaceutically acceptable salts may include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitro benzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxyburyrate, glycolate, maleate, tartrate, methanesulfonate, propanesulfonate, naphthalene-1-sulfonate, naphthalene-2-sulfonate, and mandelate. The acid addition salt may be prepared by a conventional method, for example, by dissolving a derivative of Chemical Formula 1 in an organic solvent such as methanol, ethanol, acetone, methylene chloride, or acetonitrile, adding an organic acid or inorganic acid, and filtering and drying the resulting precipitate, or prepared by distilling the solvent and excess acid under reduced pressure, dehydrating the resulting product and crystallizing the dehydrated product in an organic solvent. In addition, the pharmaceutically acceptable salt may be a salt or metal salt obtained using a base. As an example of the metal salt, an alkali metal or alkaline earth metal salt may be obtained by dissolving the compound in an excess alkali metal hydroxide or alkaline earth metal hydroxide solution, filtering an undissolved compound salt, and evaporating and dehydrating the filtrate. As an alkali metal salt, a sodium, potassium, or calcium salt may be pharmaceutically acceptable. In addition, corresponding salts may be obtained by reacting an alkali metal or alkaline earth metal salt with a suitable silver salt (e.g., silver nitrate).

Further, the present invention may include not only the compound represented by Chemical Formula 1 and a pharmaceutically acceptable salt thereof, but also an isomer thereof, especially a stereoisomer or enantiomer, or a hydrate and/or solvate that can be prepared therefrom.

The present invention may also provide a method of preparing the compound of Chemical Formula 1.

The method of preparing the compound of Chemical Formula 1 may include
preparing a compound of Chemical Formula 3 from a compound of Chemical Formula 2 (Step 1); and
preparing the compound of Chemical Formula 1 from the compound of Chemical Formula 3 (Step 2).

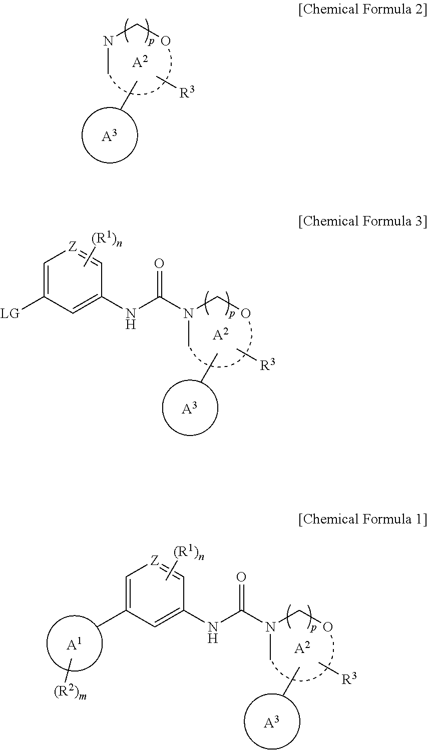

In the above Formulas, LG is a leaving group, and Z, rings A¹ to A³, R¹ to R³, m, n, and p may each be independently the same as defined in this specification. The leaving group may be a functional group such as a halogen, sulfonic acid ester, boronic ester, boronic acid, or alkoxy, and it is not limited as long as it is a functional group capable of producing the compound of Chemical Formula 1 by being detached from the compound of Chemical Formula 3.

In the method of preparing the compound of Chemical Formula 1 of the present invention, Step 1 may be a step of preparing the compound of Chemical Formula 3 by reacting the compound of Chemical Formula 2 with

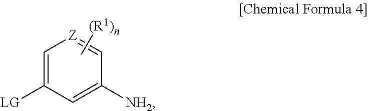

and LG-C(═O)-LG. Here, the leaving group (LG) is not limited as long as it is a functional group that can produce the compound of Chemical Formula 3 by being detached through the reaction of the compound of Chemical Formula 2 and the compound of Chemical Formula 4, and is preferably alkoxy or halogen.

In the method of preparing the compound of Chemical Formula 1 of the present invention, Step 2 may include substituting the LG group of the compound of Chemical Formula 3 with boronic ester or dioxaborolane, and reacting the resulting product with (R²)ₘ-A¹-LG (LG may be halogen). Step 2 may be to link ring A¹ with a

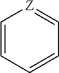

group through the Suzuki coupling reaction.

The present invention may also provide a pharmaceutical composition for preventing or treating one or more kinase-related diseases, which includes the compound, an isomer thereof, a solvate thereof, a hydrate thereof, or a pharmaceutically acceptable salt thereof as an active ingredient.

The compound of Chemical Formula 1 of the present invention may exhibit excellent inhibitory activity against various kinases, particularly, receptor-interacting serine/threonine-protein kinase 1 (RIPK1). In addition, the compound of Chemical Formula 1 may effectively inhibit cell necrosis, particularly, cell necrosis caused by necroptosis.

In the present invention, the disease may be a disease/disorder that may be at least partially regulated by programmed necrosis, apoptosis, or production of an inflammatory cytokine, particularly, inflammatory bowel disease (including Crohn's disease and ulcerative colitis), psoriasis, retinal detachment (and degeneration), retinitis pigmentosa, macular degeneration, pancreatitis, atopic dermatitis, arthritis (including rheumatoid arthritis, spondyloarthritis, gout, juvenile idiopathic arthritis (systemic juvenile idiopathic arthritis (SoJIA)), and psoriatic arthritis), systemic lupus erythematosus (SLE), Sjogren's syndrome, systemic scleroderma, anti-phospholipid syndrome (APS), vasculitis, osteoarthritis, a liver injury/disease (non-alcoholic steatohepatitis, alcoholic steatohepatitis, autoimmune hepatitis, autoimmune hepatobiliary disease, primary sclerosing cholangitis (PSC), acetaminophen toxicity, or hepatotoxicity), kidney injury/damage (nephritis, kidney transplant, surgery, administration of a nephrotoxic drug, such as cisplatin, or acute kidney injury (AKI)), celiac disease, autoimmune idiopathic thrombocytopenia purpura (autoimmune ITP), transplant rejection (rejection of a transplanted organ, tissue, or cells), ischemia-reperfusion injury of parenchymal organs, sepsis, systemic inflammatory response syndrome (SIRS), cerebrovascular accident (CVA or stroke), myocardial infarction (MI), atherosclerosis, Huntington's disease, Alzheimer's disease, Parkinson's disease, amyotrophic lateral sclerosis (ALS), neonatal hypoxic brain injury, ischemic brain injury, traumatic brain injury, allergic disease (including asthma and atopic dermatitis), burns, multiple sclerosis, type I diabetes, Wegener's granulomatosis, pulmonary sarcoidosis, Behcet's disease, interleukin-1 converting enzyme (ICE, also known as caspase-1)-associated fever syndrome, chronic obstructive pulmonary disease (COPD), cigarette smoke-induced injury, cystic fibrosis, tumor necrosis factor receptor-associated periodic syndrome (TRAPS), neoplastic tumors, periodontitis, NEMO mutation (mutation in NF-kappa-B essential regulator gene (also known as IKK gamma or IKKG)), particularly, NEMO-deficient syndrome, HOIL-1 deficiency (also known as RBCK1), heme-oxidized IRP2 ubiquitin ligase-1 deficiency), linear ubiquitin chain assembly complex (LUBAC)-deficient syndrome, blood and parenchymal malignancies, bacterial and viral infections (e.g., influenza, staphylococcus, and mycobacterium (tuberculosis)), and a lysosomal storage disease (particularly, Gaucher disease, GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile sialic acid storage disease, juvenile hexosaminidase A deficiency, Krabbe disease, lysosomal acid lipid deficiency, metachromatic leukodystrophy, mucopolysaccharidosis, multiple sulfatase deficiency, Niemann-Pick disease, neuronal ceroid lipofuscinose, Pompe disease, pyknodysostosis, Sandhoff disease, Shindler disease, sialic acid storage disease, Tay-Sachs' disease, or Wolman's disease), Stevens-Johnson syndrome, glaucoma, spinal cord injury, mesothelioma, melanoma, or acute liver failure, but the present invention is not limited thereto.

More specifically, the disease may be selected from the group consisting of inflammatory bowel disease, Crohn's disease, ulcerative colitis, psoriasis, retinal detachment, retinitis pigmentosa, macular degeneration, pancreatitis, atopic dermatitis, rheumatoid arthritis, spondyloarthritis, gout, Sjogren's syndrome, systemic scleroderma, anti-phospholipid syndrome, vasculitis, osteoarthritis, non-alcoholic steatohepatitis, alcoholic steatohepatitis, primary sclerosing cholangitis, nephritis, celiac disease, transplant rejection, sepsis, systemic inflammatory response syndrome, myocardial infarction, Huntington's disease, Alzheimer's disease, Parkinson's disease, allergic disease, asthma, atopic dermatitis, multiple sclerosis, type I diabetes, Wegener's granulomatosis, pulmonary sarcoidosis, Behcet's disease, motor neurone disease, chronic obstructive pulmonary disease, and periodontitis, but the present invention is not limited thereto.

The pharmaceutical composition of the present invention may inhibit the expression or activity of RIPK1 kinase.

The pharmaceutical composition of the present invention may include a pharmaceutically acceptable carrier. The pharmaceutically acceptable carrier may include diluents or excipients such as a filler, a thickening agent, a binder, a wetting agent, a disintegrant, and a surfactant, and the composition of the present invention may be formulated with them.

The pharmaceutical composition of the present invention may be used in clinical administration and prepared to be administered in a variety of oral and parenteral formulations.

Solid preparations for oral administration may include tablets, pills, powders, granules, and capsules, and such solid preparations may be prepared by mixing at least one excipient, such as starch, calcium carbonate, sucrose, lactose, or gelatin with one or more compounds. Aside from simple excipients, lubricants such as magnesium stearate, talc, etc. may also be used.

In addition, as liquid preparations for oral administration, a suspension, a liquid for internal use, an emulsion, or a syrup may be used, and a generally-used simple diluent such as water or liquid paraffin, as well as various types of excipients, for example, a wetting agent, a sweetener, a fragrance and a preservative may be included.

As preparations for parenteral administration, a sterile aqueous solution, a non-aqueous solvent, a suspension, or an emulsion may be used, and as the non-aqueous solvent or suspension, propylene glycol, polyethylene glycol, a vegetable oil such as olive oil, or an injectable ester such as ethyl oleate may be used.

In addition, parenteral administration may be performed by subcutaneous injection, intravenous injection, intramuscular injection, or intrathoracic injection. Here, for preparation into formulations for parenteral administration, the pharmaceutical composition may be prepared in a solution or suspension by mixing a compound represented by Chemical Formula 1 or a pharmaceutically acceptable salt thereof with a stabilizer or buffer in water, and prepared in unit-dose ampoules or vials. The composition may be sterilized and/or contain preservatives, stabilizers, wetting agents or emulsion accelerators, auxiliary materials such as salts and/or buffers for regulating osmotic pressure, and other therapeutically useful substances through mixing, granulating or coating according to a conventional method.

The present invention provides a method of preventing or treating a kinase-related disease, which includes administering the compound, an isomer thereof, a solvate thereof, a hydrate thereof, a pharmaceutically acceptable salt thereof, or a pharmaceutical composition including the same to a subject in need thereof at a pharmaceutically effective amount. The subject may be a mammal, including a human.

The term “pharmaceutically effective amount” used herein refers to an amount of the compound represented by Chemical Formula 1 that is effective in treatment or prevention of the kinase-related disease. Specifically, the “pharmaceutically effective amount” used herein refers to an amount sufficient for treating a disease at a reasonable benefit/risk ratio applicable for medical treatment, and an effective dosage may be determined by factors including a type of individual, severity, age, gender, a type of disease, drug activity, sensitivity to a drug, administration time, an administration route, an excretion rate, the duration of treatment, and concurrently used drugs, and other factors well known in the medical field. The pharmaceutical composition of the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without side effects, and such a dose may be easily determined by one of ordinary skill in the art. The dosage of the pharmaceutical composition of the present invention may be determined by an expert depending on various factors such as a patient's condition, age, gender, and complications. Since the active ingredient of the pharmaceutical composition of the present invention has excellent safety, the pharmaceutical composition of the present invention may be used at a dose exceeding the determined dosage.

The present invention provides a use of the compound of Chemical Formula 1, an isomer thereof, a solvate thereof, a hydrate thereof, or a pharmaceutically acceptable salt thereof for preparing a medicament used in the prevention or treatment of a kinase-related disease.

In the use and treatment method of the present invention, the compound of Chemical Formula 1, an isomer thereof, a solvate thereof, a hydrate thereof, a pharmaceutically acceptable salt thereof, and a kinase-related disease may be applied as described above.

Hereinafter, the present invention will be described in detail with reference to examples and experimental examples.

However, the following examples and experimental examples are merely provided to exemplify the present invention, and the contents of the present invention are not limited to the following examples.

### EXAMPLES

Compounds synthesized in Examples of the present invention were purified or subjected to structural analysis under the following HPLC conditions: Medium pressure liquid chromatography (MPLC) for purification; For MPLC, CombiFlash Rf+UV (TELEEDYNE ISCO) was used.

### HPLC Conditions for Analysis (ACQUITY UPLC H-Class Core System)

For the Waters UPLC system (ACQUITY UPLC PDA Detector), equipment equipped with the Waters mass QDA detector was used. The column used was Waters ACQUITY UPLC® BEH C18 (1.7 μm, 2.1×50 mm), and a column temperature was 30° C.

Mobile phase A was water containing 0.1% formic acid, and mobile phase B was acetonitrile containing 0.1% formic acid.

Gradient conditions (10-100% B for 3 min; flow rate=0.6 mL/min)

### Prep-150 LC System for Purification (Preparative-Liquid Chromatography UV Spectrometry)

For the Waters Prep 150 LC system (2545 Quaternary gradient module, 2998 Photodiode Array Detector, Fraction collector III), equipment manufactured by Waters was used. The column used was Waters XTERRA® Prep RP18 OBD™ (10 μm, 30×300 mm), and a column temperature was room temperature.

Gradient conditions (3-100% B for 120 min; flow rate=40 mL/min)

Commercial reagents used were used without further purification. In the present invention, room temperature is approximately 1 to 35° C. For concentration under reduced pressure or distillation for removing a solvent, a rotary evaporator was used.

### <Preparation Example 1> Preparation of(S)-3-phenylisoxazolidine

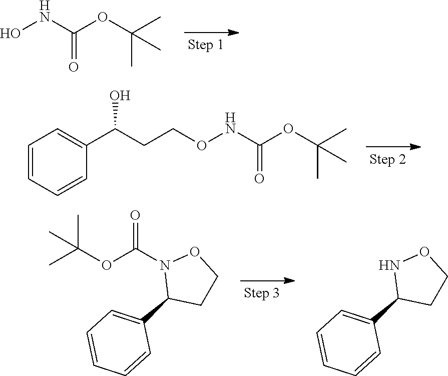

### Step 1: Preparation of tert-butyl (R)-(3-hydroxy-3-phenylpropoxy)carbamate

After dissolving tert-butyl hydroxycarbamate (7.8 g, 58.6 mmol) in dimethylformamide (DMF, 140 mL), NaH (2.58 g, 64.5 mmol) was added at 0° C. and reacted for 30 minutes. Then, (R)-3-chloro-1-phenylpropan-1-ol (5 g, 29.3 mmol) dissolved in DMF (10 mL) was slowly added dropwise for 10 minutes at 0° C. and stirred at room temperature for 72 hours. The reaction was terminated by adding an aqueous ammonium chloride solution to the reaction mixture, the resulting mixture was extracted with ethyl acetate (EA) and brine, and then organic layers were combined. The organic layer was dried over sodium sulfate, concentrated under reduced pressure, and purified by MPLC (ethyl acetate/hexane, EA/Hex), thereby obtaining the target compound (2.8 g, 68%).

MS (m/z): 150.17 [M+1]⁺

¹H NMR (400 MHZ, CDCl₃) δ=7.43-7.39 (m, 2H), 7.38-7.32 (m, 2H), 7.27-7.24 (m, 1H), 5.05-4.97 (m, 1H), 4.15-4.08 (m, 1H), 4.07-4.00 (m, 1H), 2.10-1.93 (m, 2H), 1.54-1.48 (m, 9H)

### Step 2: Preparation of tert-butyl(S)-3-phenylisoxazolidine-2-carboxylate

Tert-butyl (R)-(3-hydroxy-3-phenylpropoxy)carbamate (2.55 g, 9.54 mmol) obtained in Step 1 of Preparation Example 1 and triethylamine (TEA, 3.13 mL, 22.44 mmol) were dissolved in dichloromethane (DCM, 250 mL) and cooled to 0° C. Then, methanesulfonyl chloride (1 mL, 13 mmol) was added dropwise and stirred at 0° C. for 2 hours. The reaction mixture was extracted with brine and DCM, and then organic layers were combined. The organic layer was dried over sodium sulfate, concentrated under reduced pressure to obtain the target compound, and then the obtained compound was used in the next reaction without purification.

MS (m/z): 194.13 [M+1]⁺

¹H NMR (400 MHZ, CDCl₃) δ=7.29-7.23 (m, 4H), 7.18-7.17 (m, 1H), 5.12-5.11 (m, 1H), 4.10-4.03 (m, 1H), 3.82-3.80 (m, 1H), 2.75-2.65 (m, 1H), 2.29-2.15 (m, 1H), 1.37 (s, 9H)

### Step 3: Preparation of(S)-3-phenylisoxazolidine

Tert-butyl (S)-3-phenylisoxazolidine-2-carboxylate (2.3 g) obtained in Step 2 of Preparation Example 1 was dissolved in DCM (90 mL), and trifluoroacetic acid (TFA, 14 mL) was added and reacted at room temperature for 1 hour. The reaction mixture was neutralized with an NaHCO₃ aqueous solution, and then organic layers were combined. The organic layer was dried over sodium sulfate, concentrated under reduced pressure, and purified by MPLC (tetrahydrofuran/hexane, THF/Hex), thereby obtaining the target compound (1.3 g, 94%).

MS (m/z): 150.08 [M+1]⁺

¹H NMR (400 MHZ, DMSO-d₆) δ=7.59-7.52 (m, 2H), 7.50-7.39 (m, 3H), 5.01-4.93 (m, 1H), 2.93-2.82 (m, 1H), 2.62-2.53 (m, 2H)

### <Preparation Example 2> Preparation of (S)-3-(3-fluorophenyl)isoxazolidine

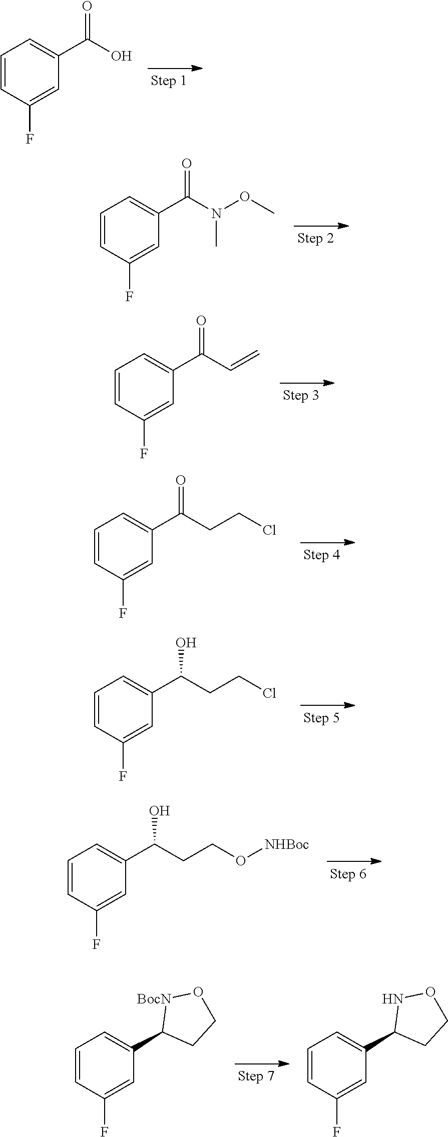

### Step 1: Preparation of 3-fluoro-N-methoxy-N-methylbenzamide

After dissolving 3-fluorobenzoic acid (90 g, 642.35 mmol, 1 eq) in pyridine (150 mL), N,O-dimethylhydroxylamine hydrochloride (75.19 g, 770.81 mmol, 1.2 eq) was added. Afterward, 1-ethyl-3-(3-dimethylaminopropyl) carbodiimide (EDCI, 147.77 g, 770.81 mmol, 1.2 eq) was added at 15° C. The reaction mixture was stirred at 50° C. for 30 minutes. As a result of TLC analysis (petroleum ether (PE):EA=3:1), all starting materials disappeared and a new spot with low polarity was detected. The pyridine solvent was removed by concentration under reduced pressure, and an organic layer was extracted using DCM (500 mL), 2N HCl (500 mL), and brine (200 mL). The organic layer was dried over sodium sulfate and concentrated under reduced pressure, thereby obtaining the target compound as a yellow oil (110 g, yield: 93.5%).

### Step 2: Preparation of 1-(3-fluorophenyl)prop-2-en-1-one

After dissolving the 3-fluoro-N-methoxy-N-methylbenzamide (110 g, 600.50 mmol, 1 eq) obtained in Step 1 of Preparation Example 2 in THF (1 L), at 0° C., bromo (vinyl) magnesium (1 M, 630.53 mL, 1.05 eq) was added dropwise at −78° C. Afterward, the reaction mixture was stirred at 0° C. for 30 minutes. As a result of TLC analysis (PE:EA=4:1), all starting materials disappeared and a new spot with low polarity was detected. After terminating the reaction by adding an HCl aqueous solution (4N, 500 mL), an organic layer was extracted using methyl tert-butyl ether (MTBE, 2000 mL) and brine (500 mL). The organic layer was dried over sodium sulfate and concentrated under reduced pressure. The concentrated compound was purified by MPLC (PE/EA=30/1), thereby obtaining the target compound as a yellow oil (80 g, 532.80 mmol, yield: 88.73%).

### Step 3: Preparation of 3-chloro-1-(3-fluorophenyl)propane-1-one

After dissolving the 1-(3-fluorophenyl)prop-2-en-1-one (71 g, 472.86 mmol, 1.0 eq) obtained in Step 2 of Preparation Example 2 in DCM (71 mL), HCl in 1,4-dioxane (4 M, 295.54 mL, 2.5 eq) was added at 0° C. Afterward, the reaction mixture was stirred at 15° C. for 1.5 hours. As a result of TLC analysis (PE:EA=10:1), all starting materials disappeared, and the target compound was detected. The reaction mixture was concentrated under reduced pressure, an organic layer was extracted by adding DCM (450 mL) and water (200 mL*5), dried over sodium sulfate, and concentrated under reduced pressure, thereby obtaining the target compound as a yellow solid (73 g, yield: 82.7%).

¹H NMR (400 MHZ, CDCl₃) δ=7.78-7.72 (m, 1H), 7.69-7.60 (m, 1H), 7.53-7.44 (m, 1H), 7.37-7.24 (m, 1H), 3.93 (t, J=6.8 Hz, 2H), 3.46 (t, J=6.8 Hz, 2H)

### Step 4: Preparation of (R)-3-chloro-1-(3-fluorophenyl)propane-1-ol

After dissolving(S)-1-methyl-3,3-diphenyltetrahydro-1H,3H-pyrrolo[1,2-c][1,3,2]oxazaborol (8.61 g, 31.08 mmol, 9.27 mL, 0.1 eq) in THF (380 mL), BH₃·THF (1 M, 186.48 mL, 0.6 eq) was added dropwise under a nitrogen stream at 0° C. The reaction mixture was stirred at 0° C. for 30 minutes. Afterward, after diluting the reaction mixture in THF (390 mL), the 3-chloro-1-(3-fluorophenyl)propan-1-one (70 g, 375.11 mmol, 1 eq) obtained in Step 3 of Preparation Example 2 was added dropwise at 0° C. The reaction mixture was stirred at 0° C. for 30 minutes. As a result of TLC analysis (PE:EA=5:1), all starting materials disappeared and a target compound spot was detected. After terminating the reaction by adding methanol (MeOH, 100 mL) at 0° C., the solvent was concentrated under reduced pressure. The organic layer of the concentrated compound was extracted using DCM (100 mL*3) and an NH₄Cl solution (300 mL). The organic layer was dried over sodium sulfate and concentrated under reduced pressure. The concentrated compound was purified by MPLC (PE:EA=50:1 to 5:1), thereby obtaining the target compound as colorless oil (55 g, yield: 92%).

MS (m/z): 135.2 [M-56+H]⁺

¹H NMR (400 MHZ, CDCl₃) δ=7.35-7.28 (m, 1H), 7.19-7.04 (m, 2H), 7.01-6.91 (m, 1H), 5.00-4.88 (m, 1H), 3.76-3.70 (m, 1H), 3.60-3.51 (m, 1H), 2.24-2.13 (m, 1H), 2.12-1.98 (m, 2H)

### Step 5: Preparation of tert-butyl (R)-(3-(3-fluorophenyl)-3-hydroxypropoxy)carbamate

After dissolving tert-butyl hydroxycarbamate (40.76 g, 306.16 mmol, 1.05 eq) in DMF (400 mL), NaH (12.83 g, 320.74 mmol, 60% purity, 1.1 eq) was added under a nitrogen stream at 0° C. The reaction mixture was stirred at 10° C. for 1 hour, the (R)-3-chloro-1-(3-fluorophenyl)propan-1-ol (55 g, 291.58 mmol, 1 eq) obtained in Step 4 of Preparation Example 2, which had been diluted in DMF (150 mL), was added dropwise at 0° C., and stirred at 10° C. for 16 hours. As a result of TLC analysis (PE:EA=2:1), all starting materials disappeared and the target compound was detected. After terminating the reaction by adding water (800 mL), the resulting solid was filtered. The obtained solid was treated with EA (800 mL), water (100 mL), and brine (100 mL) to extract an organic layer. The organic layer was dried over sodium sulfate and concentrated under reduced pressure, thereby obtaining the target compound as a light yellow solid (72 g, yield: 65%).

MS (m/z): 167.9 [M-118+H]⁺

¹H NMR (400 MHZ, CDCl₃) δ=7.17-7.06 (m, 1H), 7.01-6.93 (m, 2H), 6.80-6.73 (m, 1H), 4.89-4.79 (m, 1H), 3.95-3.89 (m, 1H), 3.88-3.82 (m, 1H), 1.89-1.81 (m, 1H), 1.80-1.69 (m, 1H), 1.32 (s, 9H)

### Step 6: Preparation of tert-butyl(S)-3-(3-fluorophenyl)isoxazolidine-2-carboxylate

After dissolving the tert-butyl (R)-(3-(3-fluorophenyl)-3-hydroxypropoxy)carbamate (72 g, 252.36 mmol, 1 eq) obtained in Step 5 of Preparation Example 2 and TEA (76.61 g, 757.07 mmol, 105.38 mL, 3 eq) in DCM (700 mL), anhydrous methanesulfonic acid (65.94 g, 378.53 mmol, 1.5 eq) was slowly added at 0° C. The reaction mixture was stirred at 20° C. for 12 hours. As a result of TLC analysis (PE:EA=3:1), all starting materials disappeared and a new spot was detected. After terminating the reaction by adding water (1000 mL), an organic layer was extracted using DCM (200 mL*3). The organic layer was dried over sodium sulfate and concentrated under reduced pressure. The concentrated compound was purified by MPLC (PE:EA=50:1 to 5:1) to extract 35 g of the target compound with an ee value of 57.7%. The target compound was purified through SFC (column: DAICEL CHIRALPAK AD-H (250 mm*30 mm, 5 μm); mobile phase: [Neu-MeOH]; B %: 15%, 2.9 min; 760 min), thereby obtaining the target compound as a light yellow solid (25 g, yield: 36.2%) and tert-butyl (R)-3-(3-fluorophenyl)isoxazolidine-2-carboxylate (6.5 g, yield: 8.7%).

MS (m/z): 212.2 [M-56+H]⁺

¹H NMR (400 MHZ, CDCl₃) δ=7.34-7.27 (m, 1H), 7.17-7.05 (m, 2H), 6.99-6.91 (m, 1H), 5.20 (dd, J=5.6, 8.8 Hz, 1H), 4.22-4.14 (m, 1H), 3.94-3.84 (m, 1H), 2.84-2.73 (m, 1H), 2.34-2.22 (m, 1H), 1.47 (s, 9H)

### Step 7: Preparation of(S)-3-(3-fluorophenyl)isoxazolidine

After dissolving the tert-butyl(S)-3-(3-fluorophenyl)isoxazolidine-2-carboxylate (25 g, 93.53 mmol, 1 eq) obtained in Step 6 of Preparation Example 2 in EA (50 mL), HCl in EA (4M, 250 mL) was added at 0° C. Afterward, the reaction mixture was stirred at 10° C. for 1 hour. As a result of LC-MS analysis, all starting materials disappeared and 96.97% of the target compound was detected. The target compound (19.12 g, yield: 93%) was obtained as a white solid by concentration under reduced pressure for obtaining a solid.

MS (m/z): 168.3 [M+H]⁺

¹H NMR (400 MHZ, DMSO-d₆) δ=12.51 (br s, 1H), 7.55-7.44 (m, 2H), 7.44-7.37 (m, 1H), 7.32-7.22 (m, 1H), 5.03 (t, J=8.0 Hz, 1H), 4.54-4.43 (m, 1H), 4.34-4.24 (m, 1H), 2.95-2.83 (m, 1H), 2.64-2.52 (m, 1H)

The compounds of Preparation Examples 3 to 33 were prepared in a similar manner to Preparation Examples 1 and 2, and the names, chemical structure formulas, and LC-MS analysis results for the compounds of Preparation Examples 3 to 33 are summarized in Table 1.

TABLE 1

| Preparation | | | LC-MS |
|---|---|---|---|
| Example | Structural Formula | Name of Compound | [M + H]⁺ |
| 3 | 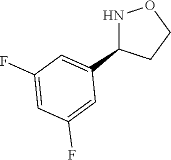 [SMILES: Fc1cc(F)cc([C@@H]2CCON2)c1] | (S)-3-(3,5-difluorophenyl)isoxazolidine | 186.1 |
| 4 | 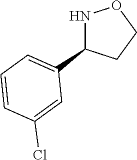 [SMILES: Clc1cccc([C@@H]2CCON2)c1] | (S)-3-(3-chlorophenyl)isoxazolidine | 184.1 |
| 5 | 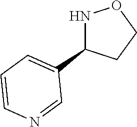 [SMILES: c1cncc([C@@H]2CCON2)c1] | (S)-3-(pyridin-3-yl)isoxazolidine | 151.1 |
| 6 | 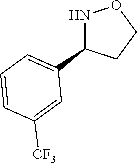 [SMILES: FC(F)(F)c1cccc([C@@H]2CCON2)c1] | (S)-3-(3-(trifluoromethyl)phenyl)isoxazolidine | 218.1 |
| 7 | 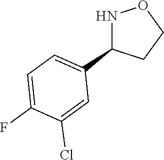 [SMILES: Fc1ccc([C@@H]2CCON2)cc1Cl] | (S)-3-(3-chloro-4-fluorophenyl)isoxazolidine | 202.0 |
| 8 | 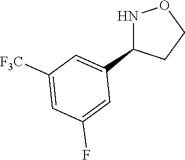 [SMILES: Fc1cc([C@@H]2CCON2)cc(C(F)(F)F)c1] | (S)-3-(3-fluoro-5-(trifluoromethyl)phenyl)isoxazolidine | 236.1 |
| 9 | 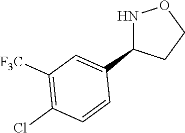 [SMILES: FC(F)(F)c1cc([C@@H]2CCON2)ccc1Cl] | (S)-3-(4-chloro-3-(trifluoromethyl)phenyl)isoxazolidine | 252.0 |
| 10 | 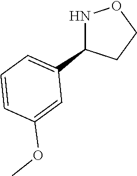 | (S)-3-(3-methoxyphenyl)isoxazolidine | 180.1 |
| 11 | 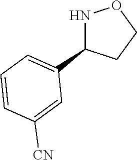 [SMILES: N#Cc1cccc([C@@H]2CCON2)c1] | (S)-3-(isoxazolidine-3-yl)benzonitrile | 175.1 |
| 12 | 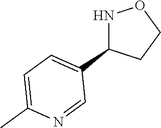 | (S)-3-(6-methylpyridin-3-yl)isoxazolidine | 165.1 |
| 13 | 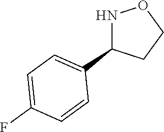 [SMILES: Fc1ccc([C@@H]2CCON2)cc1] | (S)-3-(4-fluorophenyl)isoxazolidine | 168.1 |
| 14 | 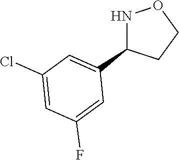 [SMILES: Fc1cc(Cl)cc([C@@H]2CCON2)c1] | (S)-3-(3-chloro-5-fluorophenyl)isoxazolidine | 202.0 |
| 15 | 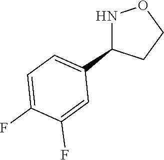 [SMILES: Fc1ccc([C@@H]2CCON2)cc1F] | (S)-3-(3,4-difluorophenyl)isoxazolidine | 186.1 |
| 16 | 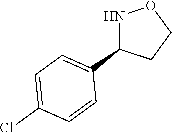 [SMILES: Clc1ccc([C@@H]2CCON2)cc1] | (S)-3-(4-chlorophenyl)isoxazolidine | 184.1 |
| 17 | 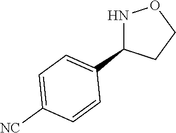 [SMILES: N#Cc1ccc([C@@H]2CCON2)cc1] | (S)-4-(isoxazolidine-3-yl)benzonitrile | 175.1 |
| 18 | 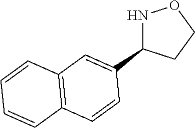 [SMILES: c1ccc2cc([C@@H]3CCON3)ccc2c1] | (S)-3-(naphthalen-2-yl)isoxazolidine | 200.1 |
| 19 | 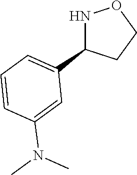 | (S)-3-(isoxazolidine-3-yl)-N,N-dimethylaniline | 193.1 |
| 20 | 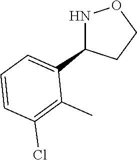 | (S)-3-(3-chloro-2-methylphenyl)isoxazolidine | 198.1 |
| 21 | 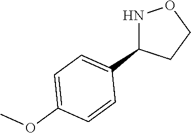 | (S)-3-(4-methoxyphenyl)isoxazolidine | 180.1 |
| 22 | 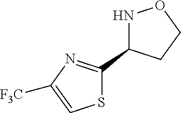 [SMILES: FC(F)(F)c1csc([C@@H]2CCON2)n1] | (S)-3-(4-(trifluoromethyl)thiazol-2-yl)isoxazolidine | 225.0 |
| 23 | 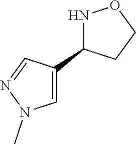 | (S)-3-(1-methyl-1H-pyrazol-4-yl)isoxazolidine | 154.1 |
| 24 | 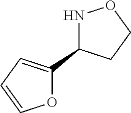 | (S)-3-(furan-2-yl)isoxazolidine | 140.1 |
| 25 | 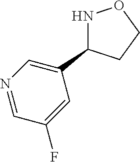 [SMILES: Fc1cncc([C@@H]2CCON2)c1] | (S)-3-(5-fluoropyridin-3-yl)isoxazolidine | 169.1 |
| 26 | 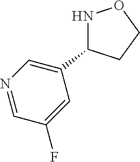 [SMILES: Fc1cncc([C@H]2CCON2)c1] | (R)-3-(5-fluoropyridin-3-yl)isoxazolidine | 169.1 |
| 27 | 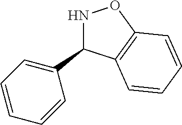 [SMILES: c1ccc([C@H]2NOc3ccccc32)cc1] | (R)-3-phenyl-2,3-dihydrobenzo[d]isoxazole | 198.1 |
| 28 | 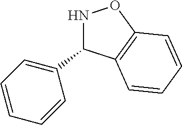 [SMILES: c1ccc([C@@H]2NOc3ccccc32)cc1] | (S)-3-phenyl-2,3-dihydrobenzo[d]isoxazole | 198.1 |
| 29 | 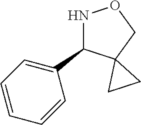 [SMILES: c1ccc([C@H]2NOCC23CC3)cc1] | (R)-7-phenyl-5-oxa-6-azaspiro[2.4]heptane | 176.1 |
| 30 | 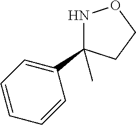 | (S)-3-methyl-3-phenylisoxazolidine | 164.1 |
| 31 | 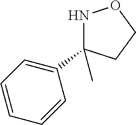 | (R)-3-methyl-3-phenylisoxazolidine | 164.1 |
| 32 | 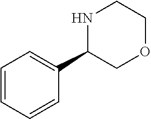 [SMILES: c1ccc([C@@H]2COCCN2)cc1] | (R)-3-phenylmorpholine | 164.1 |
| 33 | 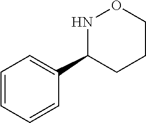 [SMILES: c1ccc([C@@H]2CCCON2)cc1] | (S)-3-phenyl-1,2-oxazinane | 164.1 |

### <Example 1> Preparation of (S)—N-(3-(2-acetamidoimidazo[1,2-b]pyrimidazin-6-yl)phenyl)-3-phenylisoxazolidine-2-carboxamide

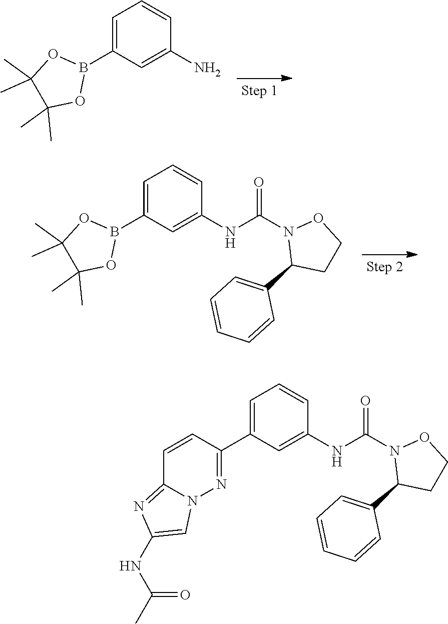 [SMILES: CC1(C)OB(c2cccc(N)c2)OC1(C)C ; CC1(C)OB(c2cccc(NC(=O)N3OCC[C@H]3c3ccccc3)c2)OC1(C)C ; CC(=O)Nc1cn2nc(-c3cccc(NC(=O)N4OCC[C@H]4c4ccccc4)c3)ccc2n1]

### Step 1: Preparation of (S)-3-phenyl-N-(3-(4,4,5,5-tetramethyl-1,3,2-dioxaborolan-2-yl)phenyl)isoxazolidine-2-carboxamide

After dissolving 3-(4,4,5,5-tetramethyl-1,3,2-dioxaborolan-2-yl) aniline (3 g) and TEA (1.39 g, 1 eq) in DCM (70 mL), phenyl chloroformate (2.36 g, 1.1 eq) was added at 0° C. The reaction mixture was stirred at 0° C. for 1 hour. Afterward, the (S)-3-phenylisoxazolidine obtained in Preparation Example 1 (2.45 g, 1.2 eq) and TEA (1.39 g, 1 eq) were added. After terminating the reaction by stirring at room temperature for 2 hours and adding MeOH (10 mL), an organic layer was extracted with water (200 mL*2), brine (200 mL), and DCM (200 mL*2). The organic layer was dried over sodium sulfate, concentrated under reduced pressure and purified by MPLC (EA/Hex), thereby obtaining the target compound (5.1 g, yield: 94%).

MS (m/z): 395.2 [M+H]⁺

### Step 2: Preparation of (S)—N-(3-(2-acetamidoimidazo[1,2-b]pyridazin-6-yl)phenyl)-3-phenylisoxazolidine-2-carboxamide

N-(6-iodoimidazo[1,2-b]pyridazin-2-yl) acetamide (66 mg), the (S)-3-phenyl-N-(3-(4,4,5,5-tetramethyl-1,3,2-dioxaborolan-2-yl)phenyl)isoxazolidine-2-carboxamide (95 mg, 1.1 eq) obtained in Step 1 of Example 1, KOAc (42 mg, 2 eq), and Pd(dtbpf)Cl₂ (14 mg, 0.1 eq) were dissolved in dioxane (0.6 mL) and water (0.15 mL) and stirred at 90° C. for 1 hour. The reaction mixture was filtered through diatomaceous earth and concentrated under reduced pressure. The target compound (32 mg, yield: 33%) was obtained through purification using the Prep-150 LC System.

### <Example 10> Preparation of (S)—N-(5-(2-(cyclopropanecarboxamido)imidazo[1,2-b]pyridazin-6-yl)-2-methylpyridin-3-yl)-3-phenylisoxazolidine-2-carboxamide

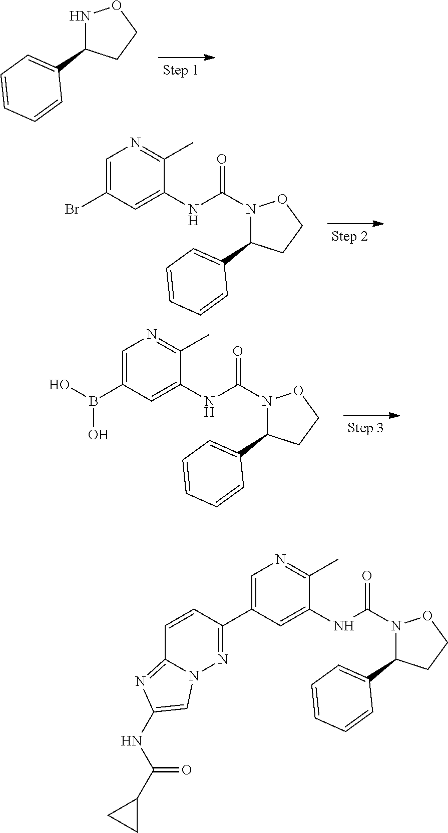

### Step 1: Preparation of(S)—N-(5-bromo-2-methylpyridin-3-yl)-3-phenylisoxazolidine-2-carboxamide

After dissolving 5-bromo-2-methylpyridine-3-amine (6 g) and pyridine (1.39 g, 1 eq) in acetonitrile (ACN) (70 mL), phenyl chloroformate (5.27 g, 1.05 eq) was added at 0° C. The reaction mixture was stirred at 0° C. for 1 hour. Afterward, the (S)-3-phenylisoxazolidine (5.03 g, 1.05 eq) obtained in Preparation Example 1 and TEA (3.57 g, 1.1 eq) were added. After terminating the reaction by stirring at room temperature for 6 hours and adding MeOH (20 mL), an organic layer was extracted using water (300 mL*2), brine (300 mL), and DCM (400 mL*2). The organic layer was dried over sodium sulfate, concentrated under reduced pressure, and purified by MPLC (EA/Hex), thereby obtaining the target compound (10.9 g, yield: 94%).

MS (m/z): 362.0 [M+H]⁺

### Step 2: Preparation of (S)-(6-methyl-5-(3-phenylisoxazolidin-2-carboxamido)pyridin-3-yl)boronic acid

The (S)—N-(5-bromo-2-methylpyridin-3-yl)-3-phenylisoxazolidine-2-carboxamide (1 g) obtained in Step 1 of Example 10, bis(pinacolato)diborone (0.84 g 1.2 eq), potassium trimethylacetate (0.78 g, 2 eq), and bis (triphenylphosphine) palladium (II) chloride (0.2 g, 0.1 eq) were dissolved in 2-methyl-tetrahydrofuran (2-MeTHF, 15 mL) and stirred at 90° C. for 2 hours. The reaction mixture was filtered through diatomaceous earth and concentrated under reduced pressure, and then the resulting product was used in the next reaction without purification.

MS (m/z): 328.1 [M+H]⁺

### Step 3: Preparation of (S)—N-(5-(2-(cyclopropanecarboxamido)imidazo[1,2-b]pyridazin-6-yl)-2-methylpyridin-3-yl)-3-phenylisoxazolidine-2-carboxamide

N-(6-iodoimidazo[1,2-b]pyridazin-2-yl) cyclopropanecarboxamide (25 mg), the (S)-(6-methyl-5-(3-phenylisoxazolidin-2-carboxamido)pyridin-3-yl)boronic acid (75 mg, 3 eq) obtained in Step 2 of Example 10, sodium carbonate (16 mg, 2 eq), and tetrakis(triphenylphosphine)palladium (0) (9 mg, 0.1 eq) were dissolved in 1,4-dioxane (0.3 mL) and water (0.1 mL) and stirred at 90° C. for 2 hours. The reaction mixture was filtered through diatomaceous earth and concentrated under reduced pressure. The target compound (10 mg, yield: 26%) was obtained through purification using the Prep-150 LC System.

Examples 1 to 158 were prepared in a similar manner to Examples 1 and 10 using Preparation Examples 1 to 33, and the names, chemical structure formulas, and NMR and LC-MS analysis results for the compounds of Examples 1 to 158 are summarized in Table 2 below.

TABLE 2

| | | | | LC-MS |
|---|---|---|---|---|
| Example | Structure | Name of Compound | ¹H NMR | [M + H]⁺ |
| 1 | 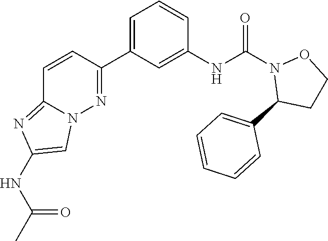 [SMILES: CC(=O)Nc1cn2nc(-c3cccc(NC(=O)N4OCC[C@H]4c4ccccc4)c3)ccc2n1] | (S)-N-(3-(2-acetamidoimidazo[1,2- b]pyridazin-6-yl)phenyl)-3- phenylisoxazolidine-2-carboxamide | ¹H NMR (400 MHz, DMSO-d₆) δ = 10.92 (s, 1H), 9.60 (s, 1H), 8.37 (t, J = 1.9 Hz, 1H), 8.28 (d, J = 0.7 Hz, 1H), 8.07 (dd, J = 9.4, 0.7 Hz, 1H), 7.82 (ddd, J = 8.2, 2.2, 1.0 Hz, 1H), 7.72-7.65 (m, 2H), 7.46 (t, J = 8.0 Hz, 1H), 7.43- 7.35 (m, 4H), 7.28 (tt, J = 6.2, 1.7 Hz, 1H), 5.44 (dd, J = 8.6, 5.9 Hz, 1H), 4.28 (td, J = 8.1, 3.1 Hz, 1H), 3.92 (ddd, J = 9.5, 8.1, 7.0 Hz, 1H), 2.87 (tdd, J = 8.7, 7.0, 3.1 Hz, 1H), 2.28-2.19 (m, 1H), 2.11 (s, 3H) | 443.3 |
| 2 | 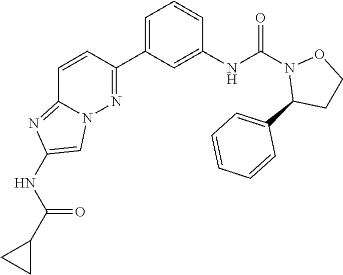 [SMILES: O=C(Nc1cn2nc(-c3cccc(NC(=O)N4OCC[C@H]4c4ccccc4)c3)ccc2n1)C1CC1] | (S)-N-(3-(2- (cyclopropanecarboxamido)imidazo[1,2- b]pyridazin-6-yl)phenyl)-3- phenylisoxazolidine-2-carboxamide | ¹H NMR (400 MHz, DMSO-d₆) δ = 11.20 (s, 1H), 9.60 (s, 1H), 8.35 (d, J = 2.0 Hz, 1H), 8.26 (s, 1H), 8.07 (d, J = 9.5 Hz, 1H), 7.85-7.80 (m, 1H), 7.71-7.65 (m, 2H), 7.45 (t, J = 7.9 Hz, 1H), 7.42-7.35 (m, 4H), 7.30-7.25 (m, 1H), 5.44 (dd, J = 8.6, 5.9 Hz, 1H), 4.27 (td, J = 8.0, 3.0 Hz, 1H), 3.92 (dt, J = 9.2, 7.3 Hz, 1H), 2.87 (tdd, J = 9.7, 7.7, 2.9 Hz, 1H), 2.29-2.20 (m, 1H), 1.97 (dq, J = 7.7, 4.9, 3.9 Hz, 1H), 0.85 (ddd, J = 9.5, 6.2, 2.5 Hz, 4H) | 469.3 |
| 3 | 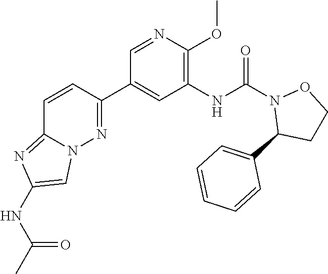 [SMILES: COc1ncc(-c2ccc3nc(NC(C)=O)cn3n2)cc1NC(=O)N1OCC[C@H]1c1ccccc1] | (S)-N-(5-(2-acetamidoimidazo[1,2- b]pyridazin-6-yl)-2-methoxypyridin-3-yl)-3- phenylisoxazolidine-2-carboxamide | ¹H NMR (400 MHz, DMSO-d₆) δ = 10.90 (s, 1H), 8.92 (d, J = 2.2 Hz, 1H), 8.56 (d, J = 2.3 Hz, 1H), 8.52 (s, 1H), 8.27 (s, 1H), 8.05 (d, J = 9.4 Hz, 1H), 7.76 (d, J = 9.5 Hz, 1H), 7.41-7.37 (m, 4H), 7.31-7.26 (m, 1H), 5.42 (dd, J = 8.5, 6.1 Hz, 1H), 4.33 (td, J = 7.8, 3.0 Hz, 1H), 4.05 (s, 3H), 4.01-3.96 (m, 1H), 2.92 (ddd, J = 8.7, 5.3, 2.5 Hz, 1H), 2.31-2.24 (m, 1H), 2.11 (s, 3H) | 474.3 |
| 4 | 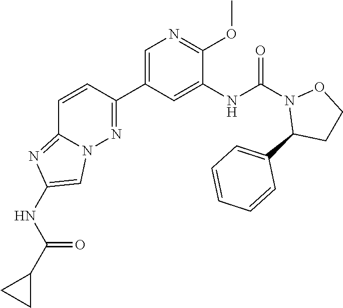 [SMILES: COc1ncc(-c2ccc3nc(NC(=O)C4CC4)cn3n2)cc1NC(=O)N1OCC[C@H]1c1ccccc1] | (S)-N-(5-(2- (cyclopropanecarboxamido)imidazo[1,2- b]pyridazin-6-yl)-2-methoxypyridin-3-yl)-3- phenylisoxazolidine-2-carboxamide | ¹H NMR (400 MHz, DMSO-d₆) δ = 11.18 (s, 1H), 8.91 (d, J = 2.3 Hz, 1H), 8.56 (d, J = 2.3 Hz, 1H), 8.52 (s, 1H), 8.24 (s, 1H), 8.05 (d, J = 9.5 Hz, 1H), 7.76 (d, J = 9.5 Hz, 1H), 7.43-7.35 (m, 4H), 7.31-7.25 (m, 1H), 5.41 (dd, J = 8.5, 6.2 Hz, 1H), 4.32 (td, J = 7.8, 3.0 Hz, 1H), 4.05 (s, 3H), 4.00 (dt, J = 9.4, 7.7 Hz, 1H), 2.92 (dddd, J = 11.9, 9.3, 6.7, 3.0 Hz, 1H), 2.28 (dq, J = 12.2, 7.6 Hz, 1H), 1.96 (td, J = 7.6, 4.2 Hz, 1H), 0.84 (tt, J = 8.2, 3.0 Hz, 4H) | 500.4 |
| 5 | 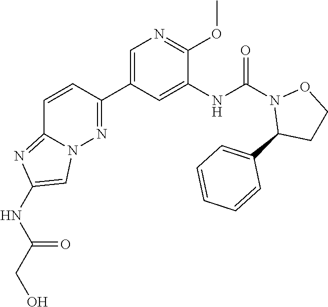 [SMILES: COc1ncc(-c2ccc3nc(NC(=O)CO)cn3n2)cc1NC(=O)N1OCC[C@H]1c1ccccc1] | (S)-N-(5-(2-(2- hydroxyacetamido)imidazo[1,2-b]pyridazin- 6-yl)-2-methoxypyridin-3-yl)-3- phenylisoxazolidine-2-carboxamide | ¹H NMR (400 MHz, DMSO-d₆) δ = 10.34 (s, 1H), 8.93 (d, J = 2.2 Hz, 1H), 8.56 (d, J = 2.3 Hz, 1H), 8.51 (s, 1H), 8.32 (s, 1H), 8.07 (d, J = 9.4 Hz, 1H), 7.77 (d, J = 9.5 Hz, 1H), 7.43-7.35 (m, 4H), 7.32-7.26 (m, 1H), 5.55 (s, 1H), 5.42 (dd, J = 8.5, 6.2 Hz, 1H), 4.33 (td, J = 7.8, 3.0 Hz, 1H), 4.14-4.08 (m, 2H), 4.05 (s, 3H), 4.04-3.97 (m, 1H), 2.92 (tdt, J = 9.3, 7.0, 3.0 Hz, 1H), 2.28 (dddd, J = 12.1, 9.6, 7.6, 6.0 Hz, 1H) | 490.3 |
| 6 | 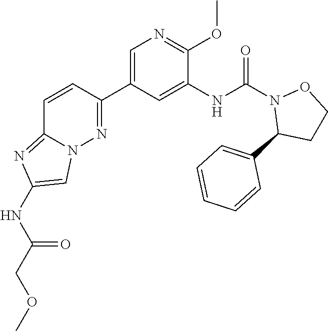 [SMILES: COCC(=O)Nc1cn2nc(-c3cnc(OC)c(NC(=O)N4OCC[C@H]4c4ccccc4)c3)ccc2n1] | (S)-N-(2-methoxy-5-(2-(2- methoxyacetamido)imidazo[1,2- b]pyridazin-6-yl)pyridin-3-yl)-3- phenylisoxazolidine-2-carboxamide | ¹H NMR (400 MHz, DMSO-d₆) δ = 10.69 (s, 1H), 8.93 (d, J = 2.2 Hz, 1H), 8.57 (d, J = 2.3 Hz, 1H), 8.52 (s, 1H), 8.32 (s, 1H), 8.07 (d, J = 9.5 Hz, 1H), 7.78 (d, J = 9.5 Hz, 1H), 7.43-7.35 (m, 4H), 7.31-7.26 (m, 1H), 5.42 (dd, J = 8.5, 6.2 Hz, 1H), 4.33 (td, J = 7.8, 3.0 Hz, 1H), 4.10 (s, 2H), 4.06 (s, 3H), 4.04-3.96 (m, 1H), 3.37 (s, 3H), 2.92 (tq, J = 8.7, 3.0 Hz, 1H), 2.28 (ddt, J = 12.3, 9.8, 7.4 Hz, 1H) | 504.3 |
| 7 | 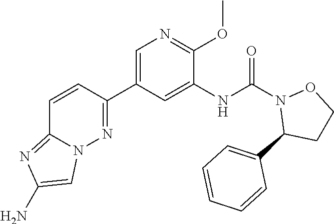 [SMILES: COc1ncc(-c2ccc3nc(N)cn3n2)cc1NC(=O)N1OCC[C@H]1c1ccccc1] | (S)-N-(5-(2-aminoimidazo[ 1,2-b]pyridazin- 6-y1)-2-methoxypyridin-3-yl)-3- phenylisoxazolidine-2-carboxamide | ¹H NMR (400 MHz, DMSO-d₆) δ = 8.88 (d, J = 2.2 Hz, 1H), 8.49 (d, J = 2.0 Hz, 2H), 7.75 (d, J = 9.2 Hz, 1H), 7.55 (d, J = 9.3 Hz, 1H), 7.41-7.36 (m, 5H), 7.29 (td, J = 6.2, 3.0 Hz, 1H), 5.41 (dd, J = 8.5, 6.2 Hz, 1H), 4.32 (td, J = 7.8, 3.0 Hz, 1H), 4.04 (s, 3H), 4.02-3.96 (m, 1H), 2.92 (tdt, J = 9.5, 7.1, 3.0 Hz, 1H), 2.32-2.22 (m, 1H) | 432.3 |
| 8 | 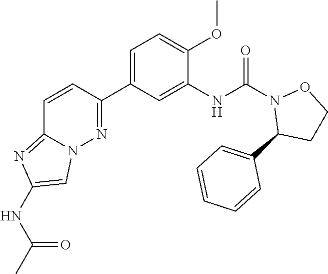 [SMILES: COc1ccc(-c2ccc3nc(NC(C)=O)cn3n2)cc1NC(=O)N1OCC[C@H]1c1ccccc1] | (S)-N-(5-(2-acetamidoimidazo[1,2- b]pyridazin-6-yl)-2-methoxyphenyl)-3- phenylisoxazolidine-2-carboxamide | ¹H NMR (400 MHz, DMSO-d₆) δ = 10.88 (s, 1H), 8.72 (d, J = 2.3 Hz, 1H), 8.59 (s, 1H), 8.24 (s, 1H), 8.01 (d, J = 9.5 Hz, 1H), 7.77 (dd, J = 8.6, 2.3 Hz, 1H), 7.67 (d, J = 9.5 Hz, 1H), 7.43-7.35 (m, 4H), 7.31-7.26 (m, 1H), 7.23 (d, J = 8.7 Hz, 1H), 5.43 (dd, J = 8.5, 6.0 Hz, 1H), 4.31 (td, J = 7.8, 3.1 Hz, 1H), 4.01-3.92 (m, 4H), 2.90 (dtt, J = 9.6, 6.9, 3.1 Hz, 1H), 2.27 (dddd, J = 12.3, 9.6, 7.8, 6.1 Hz, 1H), 2.10 (s, 3H) | 473.3 |
| 9 | 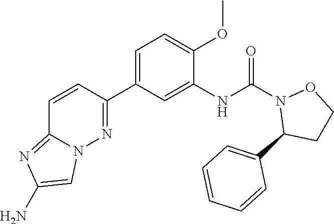 [SMILES: COc1ccc(-c2ccc3nc(N)cn3n2)cc1NC(=O)N1OCC[C@H]1c1ccccc1] | (S)-N-(5-(2-aminoimidazo[1,2-b]pyridazin- 6-yl)-2-methoxyphenyl)-3- phenylisoxazolidine-2-carboxamide | ¹H NMR (400 MHz, DMSO-d₆) δ = 8.68 (d, J = 2.2 Hz, 1H), 8.57 (s, 1H), 7.78 (d, J = 9.3 Hz, 1H), 7.70 (dd, J = 8.6, 2.3 Hz, 1H), 7.54 (d, J = 9.3 Hz, 1H), 7.41-7.36 (m, 5H), 7.30-7.26 (m, 1H), 7.21 (d, J = 8.7 Hz, 1H), 5.41 (dd, J = 8.5, 6.1 Hz, 1H), 4.31 (td, J = 7.8, 3.1 Hz, 1H), 3.96 (s, 4H), 2.90 (dddd, J = 12.0, 9.4, 6.9, 3.1 Hz, 1H), 2.26 (dddd, J = 9.5, 6.2, 4.0, 1.7 Hz, 1H) | 431.3 |
| 10 | 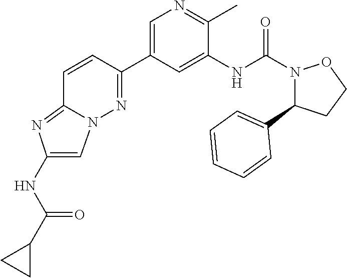 [SMILES: Cc1ncc(-c2ccc3nc(NC(=O)C4CC4)cn3n2)cc1NC(=O)N1OCC[C@H]1c1ccccc1] | (S)-N-(5-(2- (cyclopropanecarboxamido)imidazo[1,2- b]pyridazin-6-yl)-2-methylpyridin-3-yl)-3- phenylisoxazolidine-2-carboxamide | ¹H NMR (400 MHz, DMSO-d₆) δ 11.21 (s, 1H), 9.23 (s, 1H), 8.93 (t, J = 1.5 Hz, 1H), 8.38 (d, J = 2.0 Hz, 1H), 8.28 (s, 1H), 8.09 (d, J = 9.4 Hz, 1H), 7.82 (d, J = 9.5 Hz, 1H), 7.41-7.36 (m, 4H), 7.28 (s, 1H), 5.42 (t, J = 7.3 Hz, 2H), 4.30 (d, J = 8.2 Hz, 2H), 4.02 (q, J = 7.9 Hz, 2H), 2.94- 2.86 (m, 2H), 2.47 (s, 1H), 2.27 (s, 2H), 1.97 (s, 2H) | 484.2 |
| 11 | 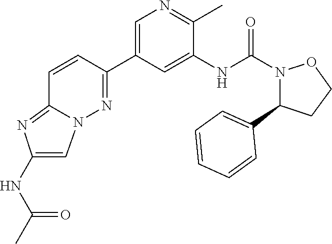 [SMILES: CC(=O)Nc1cn2nc(-c3cnc(C)c(NC(=O)N4OCC[C@H]4c4ccccc4)c3)ccc2n1] | (S)-N-(5-(2-acetamidoimidazo[1,2- b]pyridazin-6-yl)-2-methylpyridin-3-yl)-3- phenylisoxazolidine-2-carboxamide | ¹H NMR (400 MHz, DMSO-d₆) δ 10.93 (s, 1H), 9.22 (s, 1H), 8.94 (d, J = 2.2 Hz, 1H), 8.40 (d, J = 2.1 Hz, 1H), 8.31 (s, 1H), 8.09 (d, J = 9.4 Hz, 1H), 7.82 (d, J = 9.5 Hz, 1H), 7.42-7.37 (m, 4H), 7.29 (d, J = 7.0 Hz, 1H), 5.41 (d, J = 6.9 Hz, 1H), 4.31 (dd, J = 9.3, 6.5 Hz, 1H), 4.02 (d, J = 8.3 Hz, 1H), 2.90 (s, 1H), 2.47 (s, 1H), 2.29-2.25 (m, 1H), 2.17 (s, 1H), 2.11 (s, 3H), 2.06 (s, 1H) | 458.2 |
| 12 | 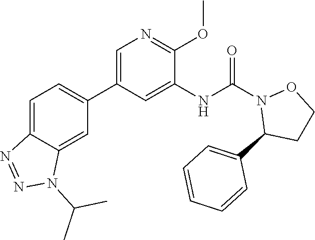 [SMILES: COc1ncc(-c2ccc3nnn(C(C)C)c3c2)cc1NC(=O)N1OCC[C@H]1c1ccccc1] | (S)-N-(5-(1-isopropyl-1H- benzo[d][1,2,3]triazol-6-yl)-2- methoxypyridin-3-yl)-3- phenylisoxazolidine-2-carboxamide | ¹H NMR (400 MHz, DMSO-d₆) δ = 8.65 (d, J = 2.3 Hz, 1H), 8.52 (s, 1H), 8.33 (d, J = 2.3 Hz, 1H), 8.13 (s, 1H), 8.11 (d, J = 8.8 Hz, 1H), 7.64 (dd, J = 8.6, 1.5 Hz, 1H), 7.40- 7.35 (m, 4H), 7.30-7.26 (m, 1H), 5.40 (dd, J = 8.5, 6.2 Hz, 1H), 5.31 (p, J = 6.7 Hz, 1H), 4.33 (td, J = 7.8, 2.9 Hz, 1H), 4.04 (s, 3H), 4.02-3.95 (m, 1H), 2.91 (dddd, J = 11.9, 9.3, 6.8, 3.0 Hz, 1H), 2.32-2.22 (m, 1H), 1.65 (d, J = 6.7 Hz, 6H) | 459.4 |
| 13 | 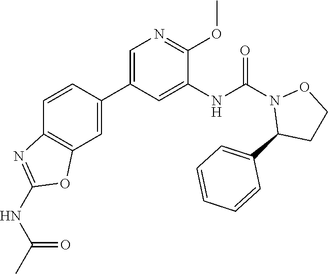 [SMILES: COc1ncc(-c2ccc3nc(NC(C)=O)oc3c2)cc1NC(=O)N1OCC[C@H]1c1ccccc1] | (S)-N-(5-(2-acetamidobenzo[d]oxazol-6-yl)- 2-methoxypyridin-3-yl)-3- phenylisoxazolidine-2-carboxamide | ¹H NMR (400 MHz, DMSO-d₆) δ = 11.70 (s, 1H), 8.57 (d, J = 2.3 Hz, 1H), 8.48 (s, 1H), 8.24 (d, J = 2.3 Hz, 1H), 7.88 (d, J= 1.7 Hz, 1H), 7.63 (d, J = 8.2 Hz, 1H), 7.53 (dd, J = 8.2, 1.7 Hz, 1H), 7.42-7.34 (m, 4H), 7.31-7.25 (m, 1H), 5.40 (dd, J = 8.5, 6.2 Hz, 1H), 4.32 (td, J = 7.8, 3.0 Hz, 1H), 4.02 (s, 3H), 4.00-3.92 (m, 1H), 2.91 (dddd, J = 11.8, 9.1, 6.7, 3.0 Hz, 1H), 2.32-2.25 (m, 1H), 2.23 (s, 3H) | 474.3 |
| 14 | 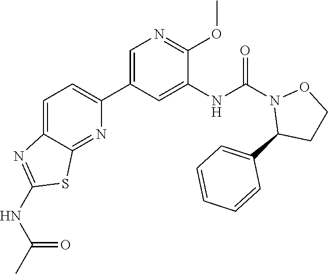 [SMILES: COc1ncc(-c2ccc3nc(NC(C)=O)sc3n2)cc1NC(=O)N1OCC[C@H]1c1ccccc1] | (S)-N-(5-(2-acetamidothiazolo[5,4- b]pyridin-5-yl)-2-methoxypyridin-3-yl)-3- phenylisoxazolidine-2-carboxamide | ¹H NMR (400 MHz, DMSO-d₆) δ = 12.49 (s, 1H), 9.00 (d, J = 2.2 Hz, 1H), 8.60 (d, J = 2.2 Hz, 1H), 8.49 (s, 1H), 8.14 (d, J = 8.6 Hz, 1H), 8.01 (d, J = 8.5 Hz, 1H), 7.39 (td, J = 8.4, 6.2 Hz, 4H), 7.31-7.26 (m, 1H), 5.42 (dd, J = 8.5, 6.2 Hz, 1H), 4.32 (td, J = 7.8, 3.0 Hz, 1H), 4.04 (s, 3H), 4.03 - 3.96 (m, 1H), 2.92 (tdt, J = 9.4, 6.9, 3.1 Hz, 1H), 2.28 (ddt, J = 11.5, 5.9, 3.9 Hz, 1H), 2.23 (s, 3H) | 491.3 |
| 15 | 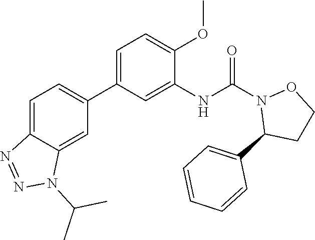 [SMILES: COc1ccc(-c2ccc3nnn(C(C)C)c3c2)cc1NC(=O)N1OCC[C@H]1c1ccccc1] | (S)-N-(5-(1-isopropyl-1H- benzo[d][1,2,3]triazol-6-yl)-2- methoxyphenyl)-3-phenylisoxazolidine-2- carboxamide | ¹H NMR (400 MHz, DMSO-d₆) δ = 8.59 (s, 1H), 8.44 (d, J = 2.4 Hz, 1H), 8.07 (d, J = 8.7 Hz, 1H), 8.02 (t, J = 1.1 Hz, 1H), 7.61 (dd, J = 8.7, 1.5 Hz, 1H), 7.51 (dd, J = 8.5, 2.4 Hz, 1H), 7.40-7.36 (m, 4H), 7.30-7.25 (m, 1H), 7.22 (d, J = 8.4 Hz, 1H), 5.42 (dd, J = 8.5, 6.1 Hz, 1H), 5.30 (dt, J = 13.6, 6.8 Hz, 1H), 4.32 (td, J = 7.9, 3.0 Hz, 1H), 3.97-3.94 (m, 4H), 2.90 (ddq, J = 11.9, 7.0, 3.1 Hz, 1H), 2.31-2.22 (m, 1H), 1.65 (dd, J = 6.7, 1.2 Hz, 6H) | 458.3 |
| 16 | 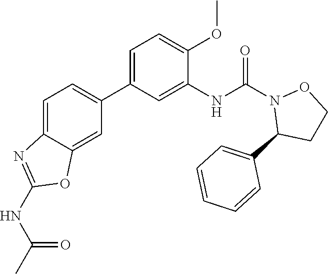 [SMILES: COc1ccc(-c2ccc3nc(NC(C)=O)oc3c2)cc1NC(=O)N1OCC[C@H]1c1ccccc1] | (S)-N-(5-(2-acetamidobenzo[d]oxazol-6-yl)- 2-methoxyphenyl)-3-phenylisoxazolidine-2- carboxamide | ¹H NMR (400 MHz, DMSO-d₆) δ = 11.67 (s, 1H), 8.56 (s, 1H), 8.36 (d, J = 2.3 Hz, 1H), 7.78 (d, J = 1.7 Hz, 1H), 7.60 (d, J = 8.3 Hz, 1H), 7.51 (dd, J = 8.3, 1.7 Hz, 1H), 7.43- 7.35 (m, 5H), 7.30-7.25 (m, 1H), 7.17 (d, J = 8.6 Hz, 1H), 5.41 (dd, J = 8.5, 6.0 Hz, 1H), 4.31 (td, J = 7.8, 3.1 Hz, 1H), 4.00-3.94 (m, 1H), 3.94 (s, 3H), 2.94-2.85 (m, 1H), 2.31-2.24 (m, 1H), 2.23 (s, 3H) | 473.3 |
| 17 | 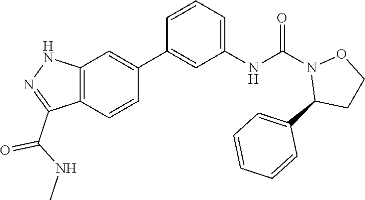 [SMILES: CNC(=O)c1n[nH]c2cc(-c3cccc(NC(=O)N4OCC[C@H]4c4ccccc4)c3)ccc12] | (S)-N-(3-(3-(methylcarbamoyl)-1H-indazol- 6-yl)phenyl)-3-phenylisoxazolidine-2- carboxamide | ¹H NMR (400 MHz, DMSO-d₆) δ 13.58 (s, 1H), 9.49 (s, 1H), 8.32 (d, J = 4.7 Hz, 1H), 8.19 (d, J = 8.5 Hz, 1H), 8.04 (s, 1H), 7.71 (s, 1H), 7.67 (d, J = 3.4 Hz, 1H), 7.48 (d, J = 8.5 Hz, 1H), 7.39-7.29 (m, 6H), 7.23 (t, J = 6.9 Hz, 1H), 5.40 (dd, J = 8.3, 6.0 Hz, 1H), 4.23 (dd, J = 7.9, 5.0 Hz, 1H), 3.88 (dd, J = 16.7, 7.8 Hz, 1H), 2.87-2.81 (m, 1H), 2.79 (d, J = 4.6 Hz, 3H), 2.25-2.11 (m, 1H) | 442.3 |
| 18 | 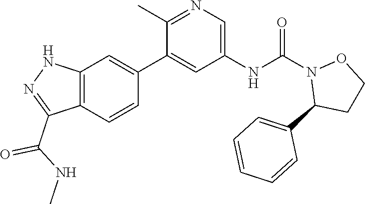 [SMILES: CNC(=O)c1n[nH]c2cc(-c3cc(NC(=O)N4OCC[C@H]4c4ccccc4)cnc3C)ccc12] | (S)-N-(6-methyl-5-(3-(methylcarbamoyl)- 1H-indazol-6-yl)pyridin-3-y1)-3- phenylisoxazolidine-2-carboxamide | ¹H NMR (400 MHz, CDCl₃) δ = 10.21 (s, 1H), 8.48 (d, J = 2.6 Hz, 1H), 8.43 (dd, J = 8.4, 0.8 Hz, 1H), 8.07 (d, J = 2.6 Hz, 1H), 7.93 (s, 1H), 7.40 (dd, J = 6.7, 1.2 Hz, 2H), 7.35 (td, J = 6.8, 6.3, 1.7 Hz, 2H), 7.29-7.27 (m, 1H), 7.25 (d, J = 2.4 Hz, 2H), 7.07-7.02 (m, 1H), 5.50 (dd, J = 8.5, 5.9 Hz, 1H), 4.29 (td, J = 7.8, 3.6 Hz, 1H), 4.03-3.96 (m, 1H), 3.08 (d, J = 5.1 Hz, 3H), 2.90-2.81 (m, 1H), 2.47 (s, 4H) | 457.3 |
| 19 | 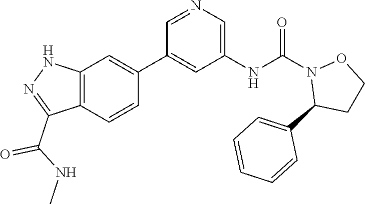 [SMILES: CNC(=O)c1n[nH]c2cc(-c3cncc(NC(=O)N4OCC[C@H]4c4ccccc4)c3)ccc12] | (S)-N-(5-(3-(methylcarbamoyl)-1H-indazol- 6-yl)pyridin-3-yl)-3-phenylisoxazolidine-2- carboxamide | ¹H NMR (400 MHz, CDCl₃) δ = 10.64 (s, 1H), 8.61 (s, 1H), 8.54 (s, 1H), 8.48-8.42 (m, 2H), 8.05 (s, 1H), 7.64 (s, 1H), 7.52 (dd, J = 8.4, 1.4 Hz, 1H), 7.44-7.40 (m, 2H), 7.37 (ddd, J = 7.8, 6.8, 1.3 Hz, 2H), 7.31-7.27 (m, 1H), 7.06 (d, J = 5.5 Hz, 1H), 5.53 (dd, J = 8.5, 6.0 Hz, 1H), 4.31 (td, J = 7.8, 3.5 Hz, 1H), 4.01 (ddd, J = 9.2, 7.9, 6.9 Hz, 1H), 3.07 (d, J = 5.0 Hz, 3H), 2.93-2.84 (m, 1H), 2.50- 2.43 (m, 1H) | 443.3 |
| 20 | 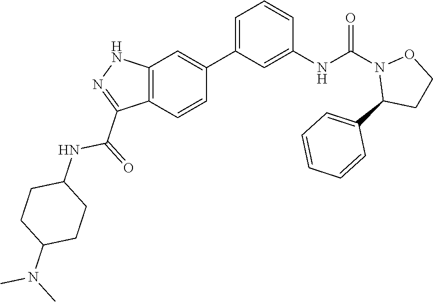 [SMILES: CN(C)C1CCC(NC(=O)c2n[nH]c3cc(-c4cccc(NC(=O)N5OCC[C@H]5c5ccccc5)c4)ccc23)CC1] | (S)-N-(3-(3-((4- (dimethylamino)cyclohexyl)carbamoyl)-1H- indazol-6-yl)phenyl)-3-phenylisoxazolidine- 2-carboxamide | ¹H NMR (400 MHz, CDCl₃) δ = 10.31 (s, 1H), 8.41 (dd, J = 8.5, 0.8 Hz, 1H), 8.01 (s, 1H), 7.93 (d, J = 2.0 Hz, 1H), 7.65 (t, J = 1.1 Hz, 1H), 7.55 (dd, J = 8.5, 1.5 Hz, 1H), 7.45-7.34 (m, 7H), 7.30-7.27 (m, 1H), 6.89 (d, J = 8.3 Hz, 1H), 5.57 (dd, J = 8.5, 5.7 Hz, 1H), 4.27 (dt, J = 7.8, 3.9 Hz, 1H), 3.99 (ddd, J = 9.0, 7.9, 7.0 Hz, 2H), 2.90-2.81 (m, 1H), 2.49- 2.40 (m, 1H), 2.34 (s, 6H), 2.24 (s, 2H), 2.17 (s, 5 H), 2.00 (d, J = 12.7 Hz, 2H) | 553.7 |
| 21 | 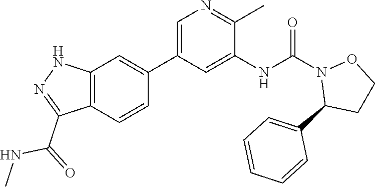 [SMILES: CNC(=O)c1n[nH]c2cc(-c3cnc(C)c(NC(=O)N4OCC[C@H]4c4ccccc4)c3)ccc12] | (S)-N-(2-methyl-5-(3-(methylcarbamoyl)- 1H-indazol-6-yl)pyridin-3-yl)-3- phenylisoxazolidine-2-carboxamide | ¹H NMR (400 MHz, CDCl₃) δ = 10.48 (s, 1H), 8.66 (d, J = 2.1 Hz, 1H), 8.55 (d, J = 2.1 Hz, 1H), 8.45 (dd, J = 8.5, 0.8 Hz, 1H), 7.92 (s, 1H), 7.66 (t, J = 1.1 Hz, 1H), 7.54 (dd, J = 8.5, 1.5 Hz, 1H), 7.46-7.41 (m, 2H), 7.40-7.34 (m, 2H), 7.32-7.28 (m, 1H), 7.04 (d, J = 5.3 Hz, 1H), 5.55 (dd, J = 8.5, 5.9 Hz, 1H), 4.34 (td, J = 7.8, 3.6 Hz, 1H), 4.09-4.00 (m, 1H), 3.07 (d, J = 5.1 Hz, 3H), 2.95-2.85 (m, 1H), 2.61 (s, 3H), 2.48 (dddd, J = 13.1, 9.1, 7.7, 5.9 Hz, 1 H) | 457.3 |
| 22 | 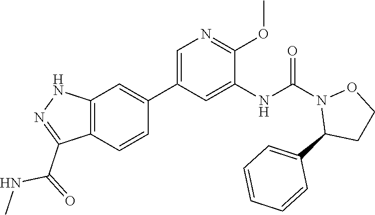 [SMILES: CNC(=O)c1n[nH]c2cc(-c3cnc(OC)c(NC(=O)N4OCC[C@H]4c4ccccc4)c3)ccc12] | (S)-N-(2-methoxy-5-(3-(methylcarbamoyl)- 1H-indazol-6-yl)pyridin-3-yl)-3- phenylisoxazolidine-2-carboxamide | ¹H NMR (400 MHz, DMSO) δ = 13.60 (s, 1H), 8.64 (d, J = 2.3 Hz, 1H), 8.49 (s, 1H), 8.36 (q, J = 4.6 Hz, 1H), 8.28 (d, J = 2.3 Hz, 1H), 8.23 (dd, J = 8.5, 0.8 Hz, 1H), 7.74 (t, J = 1.2 Hz, 1H), 7.49 (dd, J = 8.5, 1.5 Hz, 1H), 7.42-7.34 (m, 4H), 7.28 (ddt, J = 8.5, 6.0, 1.9 Hz, 1H), 5.41 (dd, J = 8.5, 6.2 Hz, 1H), 4.33 (td, J = 7.8, 3.1 Hz, 1H), 4.04 (s, 3H), 4.02-3.95 (m, 1H), 2.95-2.86 (m, 1H), 2.83 (d, J = 4.7 Hz, 3H), 2.31-2.24 (m, 1H) | 473.3 |
| 23 | 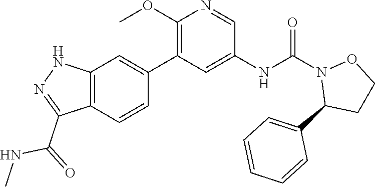 [SMILES: CNC(=O)c1n[nH]c2cc(-c3cc(NC(=O)N4OCC[C@H]4c4ccccc4)cnc3OC)ccc12] | (S)-N-(6-methoxy-5-(3-(methylcarbamoyl)- 1H-indazol-6-yl)pyridin-3-y1)-3- phenylisoxazolidine-2-carboxamide | ¹H NMR (400 MHz, DMSO) δ = 9.57 (s, 1H), 8.44 (d, J = 2.6 Hz, 1H), 8.37 (q, J = 4.7 Hz, 1H), 8.20 (d d, J = 8.5, 0.8 Hz, 1H), 8.11 (d, J = 2.6 Hz, 1H), 7.76 (t, J = 1.2 Hz, 1H), 7.42-7.32 (m, 5H), 7.30- 7.23 (m, 1H), 5.41 (dd, J = 8.6, 5.8 Hz, 1H), 4.27 (td, J = 7.9, 3.1 Hz, 1H), 4.03 (q, J = 7.1 Hz, 1H), 3.95- 3.89 (m, 1H), 3.88 (s, 3H), 2.91-2.85 (m, 1H), 2.83 (d, J = 4.7 Hz, 3H), 2.27-2.20 (m, 1H) | 473.3 |
| 24 | 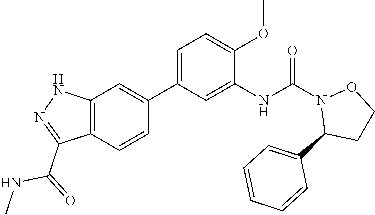 [SMILES: CNC(=O)c1n[nH]c2cc(-c3ccc(OC)c(NC(=O)N4OCC[C@H]4c4ccccc4)c3)ccc12] | (S)-N-(2-methoxy-5-(3-(methylcarbamoyl)- 1H-indazol-6-yl)phenyl)-3- phenylisoxazolidine-2-carboxamide | ¹H NMR (400 MHz, DMSO-d₆) δ = 13.51 (s, 1H), 8.56 (s, 1H), 8.44 (d, J = 2.3 Hz, 1H), 8.34 (q, J = 4.6 Hz, 1H), 8.19 (dd, J = 8.5, 0.8 Hz, 1H), 7.68 (t, J = 1.1 Hz, 1H), 7.47 (ddd, J = 10.7, 8.5, 1.9 Hz, 2H), 7.42-7.34 (m, 4H), 7.30- 7.25 (m, 1H), 7.19 (d, J = 8.6 Hz, 1H), 5.42 (dd, J = 8.5, 6.0 Hz, 1H), 4.31 (td, J = 7.8, 3.1 Hz, 1H), 4.01-3.95 (m, 1H), 3.95 (s, 3H), 2.94-2.85 (m, 1H), 2.82 (d, J = 4.7 Hz, 3H), 2.27 (ddt, J = 12.1, 9.5, 6.7 Hz, 1H) | 472.3 |
| 25 | 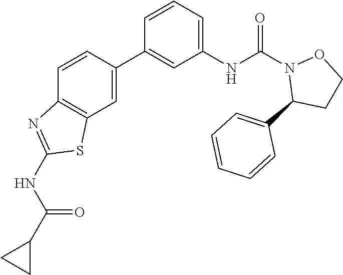 [SMILES: O=C(Nc1nc2ccc(-c3cccc(NC(=O)N4OCC[C@H]4c4ccccc4)c3)cc2s1)C1CC1] | (S)-N-(3-(2- (cyclopropanecarboxamido)benzo[d]thiazol- 6-yl)phenyl)-3-phenylisoxazolidine-2- carboxamide | ¹H NMR (400 MHz, DMSO) δ = 12.67 (s, 1H), 9.48 (s, 1H), 8.21 (d, J = 1.9 Hz, 1H), 8.06-8.02 (m, 1H), 7.79 (d, J = 8.4 Hz, 1H), 7.68 (ddd, J = 7.9, 5.3, 2.0 Hz, 2H), 7.40- 7.36 (m, 5H), 7.30-7.24 (m, 2H), 5.43 (dd, J = 8.7, 5.9 Hz, 1H), 4.27 (td, J = 7.9, 3.0 Hz, 1H), 3.95-3.87 (m, 1H), 2.87 (ddt, J = 12.4, 6.1, 3.4 Hz, 1H), 2.28-2.20 (m, 1H), 2.03-1.98 (m, 1H), 0.98-0.93 (m, 4H) | 485.3 |
| 26 | 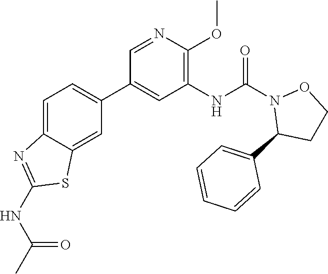 [SMILES: COc1ncc(-c2ccc3nc(NC(C)=O)sc3c2)cc1NC(=O)N1OCC[C@H]1c1ccccc1] | (S)-N-(5-(2-acetamidobenzo[d]thiazol-6- yl)-2-methoxypyridin-3-yl)-3- phenylisoxazolidine-2-carboxamide | ¹H NMR (400 MHz, DMSO) δ = 12.36 (s, 1H), 8.60 (d, J = 2.3 Hz, 1H), 8.48 (s, 1H), 8.23 (dd, J = 3.0, 2.0 Hz, 2H), 7.79 (d, J = 8.4 Hz, 1H), 7.65 (dd, J = 8.5, 1.9 Hz, 1H), 7.42-7.34 (m, 4H), 7.30-7.26 (m, 1H), 5.39 (dd, J = 8.5, 6.2 Hz, 1H), 4.33 (td, J = 7.8, 3.0 Hz, 1H), 4.03 (s, 3H), 4.01- 3.95 (m, 1H), 2.91 (dddd, J = 11.9, 9.3, 6.7, 3.0 Hz, 1H), 2.30-2.24 (m, 1H), 2.21 (s, 3H) | 490.3 |
| 27 | 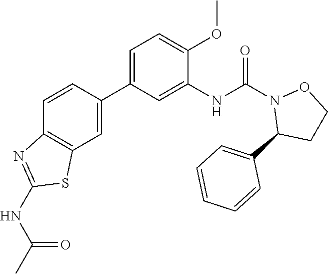 [SMILES: COc1ccc(-c2ccc3nc(NC(C)=O)sc3c2)cc1NC(=O)N1OCC[C@H]1c1ccccc1] | (S)-N-(5-(2-acetamidobenzo[d]thiazol-6- yl)-2-methoxyphenyl)-3- phenylisoxazolidine-2-carboxamide | ¹H NMR (400 MHz, DMSO-d₆) δ = 12.35 (s, 1H), 8.56 (s, 1H), 8.39 (d, J = 2.3 Hz, 1H), 8.17 (d, J = 1.9 Hz, 1H), 7.77 (d, J = 8.4 Hz, 1H), 7.62 (dd, J = 8.5, 1.9 Hz, 1H), 7.43- 7.35 (m, 5H), 7.31-7.25 (m, 1H), 7.18 (d, J = 8.6 Hz, 1H), 5.41 (dd, J = 8.5, 6.1 Hz, 1H), 4.31 (td, J = 7.8, 3.0 Hz, 1H), 4.01-3.95 (m, 1H), 3.94 (s, 3H), 2.90 (dddd, J = 11.9, 9.4, 6.9, 3.1 Hz, 1H), 2.32-2.23 (m, 1H), 2.21 (s, 3H) | 489.3 |
| 28 | 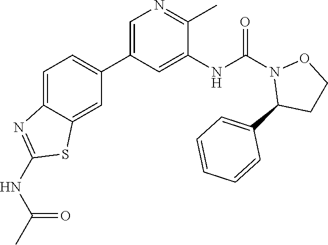 [SMILES: CC(=O)Nc1nc2ccc(-c3cnc(C)c(NC(=O)N4OCC[C@H]4c4ccccc4)c3)cc2s1] | (S)-N-(5-(2-acetamidobenzo[d]thiazol-6- yl)-2-methylpyridin-3-yl)-3- phenylisoxazolidine-2-carboxamide | ¹H NMR (400 MHz, DMSO) δ = 12.41 (s, 1H), 9.15 (s, 1H), 8.66 (s, 1H), 8.33 (s, 1H), 8.09 (s, 1H), 7.86 - 7.67 (m, 2H), 7.50 - 7.23 (m, 5H), 5.42 (m, 1H), 4.31 (m, 1H), 4.01 (m, 1H), 2.88 (m, 1H), 2.33 (m, 1H), 2.22 (s, 3H), 1.23 (s, 3H) | 474.2 |
| 29 | 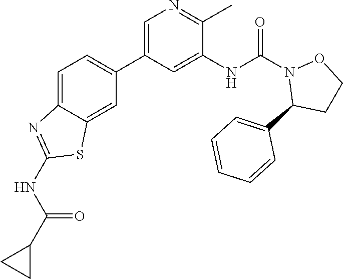 [SMILES: Cc1ncc(-c2ccc3nc(NC(=O)C4CC4)sc3c2)cc1NC(=O)N1OCC[C@H]1c1ccccc1] | (S)-N-(5-(2- (cyclopropanecarboxamido)benzo[d]thiazol- 6-yl)-2-methylpyridin-3-yl)-3- phenylisoxazolidine-2-carboxamide | ¹H NMR (400 MHz, DMSO) δ = 12.71 (s, 1H), 9.15 (s, 1H), 8.73-8.60 (m, 1H), 8.33 (s, 1H), 8.10 (s, 1H), 7.87- 7.70 (m, 2H), 7.47-7.22 (m, 5H), 5.42 (m, 1H), 4.30 (m, 1H), 4.01 (m, 1H), 2.92 (m, 1H), 2.26 (m, 2H), 2.02 (m, 1H), 1.24 (m, 2H), 0.96 (s, 3H), 0.85 (m, 1H) | 500.2 |
| 30 | 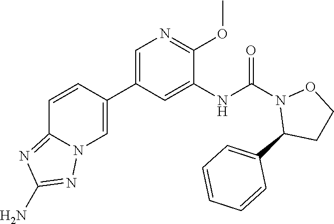 [SMILES: COc1ncc(-c2ccc3nc(N)nn3c2)cc1NC(=O)N1OCC[C@H]1c1ccccc1] | (S)-N-(5-(2-amino-[1,2,4]triazolo[1,5- a]pyridin-6-yl)-2-methoxypyridin-3-yl)-3- phenylisoxazolidine-2-carboxamide | ¹H NMR (400 MHz, DMSO-d₆) δ 8.84 (d, J = 2.2 Hz, 1H), 8.56-8.46 (m, 2H), 8.25 (t, J = 2.4 Hz, 1H), 7.67 (dt, J = 9.1, 2.2 Hz, 1H), 7.47-7.34 (m, 5H), 7.28 (td, J = 6.4, 2.3 Hz, 1H), 6.09 (s, 2H), 5.39 (ddd, J = 8.5, 6.0, 2.2 Hz, 1H), 4.36-4.29 (m, 1H), 4.05-3.94 (m, 4H), 2.91 (ddd, J = 12.3, 9.0, 6.4 Hz, 1H), 2.27 (p, J = 9.2, 8.3 Hz, 1H) | 432.2 |
| 31 | 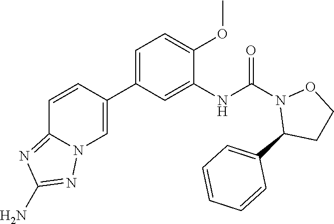 [SMILES: COc1ccc(-c2ccc3nc(N)nn3c2)cc1NC(=O)N1OCC[C@H]1c1ccccc1] | (S)-N-(5-(2-amino-[1,2,4]triazolo[1,5- a]pyridin-6-yl)-2-methoxyphenyl)-3- phenylisoxazolidine-2-carboxamide | ¹H NMR (400 MHz, DMSO-d₆) δ = 8.71 (d, J = 1.8 Hz, 1H), 8.56 (s, 1H), 8.32 (d, J = 2.3 Hz, 1H), 7.64 (dd, J = 9.2, 1.9 Hz, 1H), 7.45-7.37 (m, 5H), 7.36 (d, J = 8.0 Hz, 1H), 7.30-7.25 (m, 1H), 7.17 (d, J = 8.6 Hz, 1H), 6.04 (s, 2H), 5.41 (dd, J = 8.5, 6.1 Hz, 1H), 4.31 (td, J = 7.8, 3.1 Hz, 1H), 4.00-3.94 (m, 1H), 3.93 (s, 3H), 2.89 (dddd, J = 12.0, 8.6, 6.8, 3.1 Hz, 1H), 2.26 (dddd, J = 12.2, 9.4, 7.7, 6.0 Hz, 1H) | 431.3 |
| 32 | 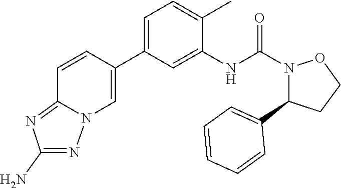 [SMILES: Cc1ccc(-c2ccc3nc(N)nn3c2)cc1NC(=O)N1OCC[C@H]1c1ccccc1] | (S)-N-(5-(2-amino-[1,2,4]triazolo[1,5- a]pyridin-6-yl)-2-methylphenyl)-3- phenylisoxazolidine-2-carboxamide | ¹H NMR (400 MHz, DMSO-d₆) δ 8.96 (s, 1H), 8.80 (s, 1H), 7.78-7.68 (m, 2H), 7.50-7.45 (m, 1H), 7.44-7.26 (m, 6H), 6.05 (s, 2H), 5.42 (dd, J = 8.7, 5.7 Hz, 1H), 4.27 (td, J = 7.9, 3.1 Hz, 1H), 4.00 (q, J = 8.1 Hz, 1H), 2.89 (dd, J = 10.6, 6.4 Hz, 2H), 2.26 (s, 4H) | 415.2 |
| 33 | 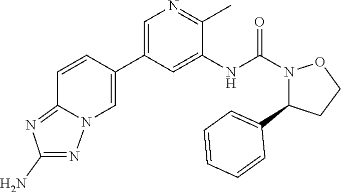 [SMILES: Cc1ncc(-c2ccc3nc(N)nn3c2)cc1NC(=O)N1OCC[C@H]1c1ccccc1] | (S)-N-(5-(2-amino-[1,2,4]triazolo[1,5- a]pyridin-6-yl)-2-methylpyridin-3-yl)-3- phenylisoxazolidine-2-carboxamide | ¹H NMR (400 MHz, DMSO-d₆) δ 9.18 (s, 1H), 8.96 (d, J = 1.9 Hz, 1H), 8.66 (d, J = 2.2 Hz, 1H), 8.08 (d, J = 2.2 Hz, 1H), 7.77 (dd, J = 9.1, 1.9 Hz, 1H), 7.45 (d, J = 9.1 Hz, 1H), 7.42-7.34 (m, 4H), 7.30-7.25 (m, 1H), 6.10 (s, 2H), 5.41 (dd, J = 8.6, 5.8 Hz, 1H), 4.30 (td, J = 7.9, 3.0 Hz, 1H), 4.00 (dt, J = 9.1, 7.4 Hz, 1H), 2.89 (tdd, J = 9.5, 7.6, 3.0 Hz, 1H), 2.47 (s, 3H), 2.30-2.22 (m, 1H) | 416.3 |
| 34 | 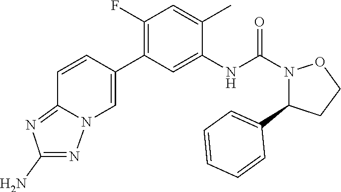 [SMILES: Cc1cc(F)c(-c2ccc3nc(N)nn3c2)cc1NC(=O)N1OCC[C@H]1c1ccccc1] | (S)-N-(5-(2-amino-[1,2,4]triazolo[1,5- a]pyridin-6-yl)-4-fluoro-2-methylphenyl)-3- phenylisoxazolidine-2-carboxamide | ¹H NMR (400 MHz, DMSO-d₆) δ 9.02 (s, 1H), 8.69 (s, 1H), 7.67-7.54 (m, 4H), 7.45 (d, J = 9.1 Hz, 1H), 7.42-7.33 (m, 4H), 7.31-7.21 (m, 2H), 5.40 (dd, J = 8.7, 5.6 Hz, 1H), 4.26 (td, J = 8.0, 3.2 Hz, 1H), 3.98 (dt, J = 9.1, 7.5 Hz, 1H), 2.87 (dtd, J = 11.7, 7.8, 3.1 Hz, 1H), 2.27 (s, 4H) | 433.2 |
| 35 | 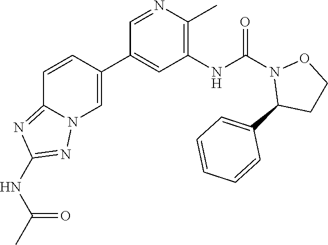 [SMILES: CC(=O)Nc1nc2ccc(-c3cnc(C)c(NC(=O)N4OCC[C@H]4c4ccccc4)c3)cn2n1] | (S)-N-(5-(2-acetamido-[1,2,4]triazolo[1,5- a]pyridin-6-yl)-2-methylpyridin-3-yl)-3- phenylisoxazolidine-2-carboxamide | ¹H NMR (400 MHz, DMSO) δ = 10.83 (s, 1H), 9.31 (s, 1H), 9.21 (s, 1H), 8.73 (s, 1H), 8.16 (s, 1H), 7.97 (s, 1H), 7.78 (d, J = 8.5 Hz, 1H), 7.39 (m, J = 9.0 Hz, 4H), 7.29 (d, J = 6.5 Hz, 1H), 5.42 (m, 1H), 4.31 (m, 1H), 4.02 (m, 1H), 2.90 (m, 1H), 2.27 (m, 1H), 2.16 (s, 3H), 1.23 (s, 3H) | 458.2 |
| 36 | 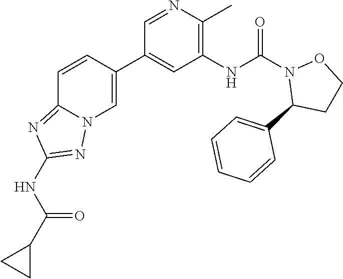 [SMILES: Cc1ncc(-c2ccc3nc(NC(=O)C4CC4)nn3c2)cc1NC(=O)N1OCC[C@H]1c1ccccc1] | (S)-N-(5-(2-(cyclopropanecarboxamido)- [1,2,4]triazolo[1,5-a]pyridin-6-yl)-2- methylpyridin-3-yl)-3-phenylisoxazolidine- 2-carboxamide | ¹H NMR (400 MHz, DMSO) δ = 11.11 (s, 1H), 9.30 (s, 1H), 9.22 (s, 1H), 8.74 (s, 1H), 8.18 (s, 1H), 7.99 (d, J = 11.0 Hz, 1H), 7.78 (d, J = 9.4 Hz, 1H), 7.50-7.18 (m, 6H), 5.47-5.38 (m, 1H), 4.31 (s, 1H), 4.07-3.96 (m, 1H), 2.93 (d, J = 16.1 Hz, 2H), 2.26 (d, J = 10.8 Hz, 1H), 2.08 (d, J = 9.8 Hz, 2H), 1.19 (s, 1H), 0.85 (s, 3H) | 484.2 |
| 37 | 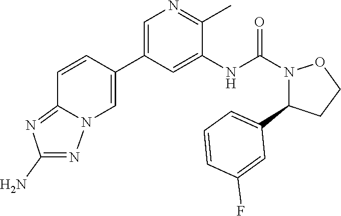 [SMILES: Cc1ncc(-c2ccc3nc(N)nn3c2)cc1NC(=O)N1OCC[C@H]1c1cccc(F)c1] | (S)-N-(5-(2-amino-[1,2,4]triazolo[1,5- a]pyridin-6-yl)-2-methylpyridin-3-yl)-3-(3- fluorophenyl)isoxazolidine-2-carboxamide | ¹H NMR (400 MHz, DMSO) δ = 9.27 (s, 1H), 9.01 (s, 1H), 8.71 (d, J = 2.4 Hz, 1H), 8.16 (s, 1H), 7.80 (d, J = 9.4 Hz, 1H), 7.48 (t, J = 8.8 Hz, 1H), 7.42 (q, J = 7.9 Hz, 1H), 7.23 (dd, J = 20.3, 8.6 Hz, 2H), 7.11 (td, J = 8.6, 2.9 Hz, 1H), 6.21 (s, 2H), 5.44 (dd, J = 8.9, 5.6 Hz, 1H), 4.31 (d, J = 3.3 Hz, 1H), 4.01 (d, J = 9.3 Hz, 1H), 2.91 (dd, J = 7.1, 3.5 Hz, 1H), 2.54 (s, 3H), 2.27 (m, 1H) | 434.3 |
| 38 | 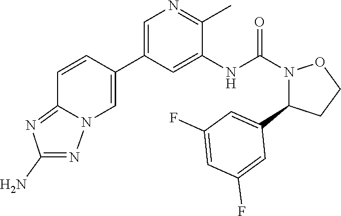 [SMILES: Cc1ncc(-c2ccc3nc(N)nn3c2)cc1NC(=O)N1OCC[C@H]1c1cc(F)cc(F)c1] | (S)-N-(5-(2-amino-[1,2,4]triazolo[1,5- a]pyridin-6-yl)-2-methylpyridin-3-yl)-3- (3,5-difluorophenyl)isoxazolidine-2- carboxamide | ¹H NMR (400 MHz, DMSO) δ = 9.26 (s, 1H), 8.97 (dd, J = 1.9, 0.9 Hz, 1H), 8.68 (d, J = 2.3 Hz, 1H), 8.08 (d, J = 2.3 Hz, 1H), 7.78 (dd, J= 9.2, 1.9 Hz, 1 H), 7.45 (dd, J = 9.1, 0.9 Hz, 1H), 7.19-7.08 (m, 3 H), 6.11 (s, 2H), 5.46 (dd, J = 8.8, 5.6 Hz, 1H), 4.29 (td, J = 7.5, 3.2 Hz, 1H), 4.00 (dt, J = 9.4, 7.1 Hz, 1 H), 2.95-2.87 (m, 1H), 2.47 (s, 3H), 2.30-2.23 (m, 1H) | 452.2 |
| 39 | 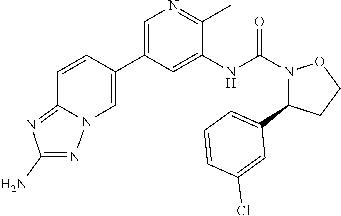 [SMILES: Cc1ncc(-c2ccc3nc(N)nn3c2)cc1NC(=O)N1OCC[C@H]1c1cccc(Cl)c1] | (S)-N-(5-(2-amino-[1,2,4]triazolo[1,5- a]pyridin-6-yl)-2-methylpyridin-3-yl)-3-(3- chlorophenyl)isoxazolidine-2-carboxamide | ¹H NMR (400 MHz, DMSO) δ = 9.22 (s, 1H), 8.97 (d, J = 2.3 Hz, 1H), 8.67 (d, J = 2.3 Hz, 1H), 8.09 (d, J = 2.4 Hz, 1H), 7.77 (dd, J = 9.1, 2.0 Hz, 1H), 7.45 (td, J = 4.6, 4.1, 2.6 Hz, 2H), 7.42-7.33 (m, 3H), 6.10 (s, 2H), 5.43 (dd, J = 8.8, 5.7 Hz, 1H), 4.30 (td, J = 8.1, 3.4 Hz, 1H), 4.00 (q, J = 7.9 Hz, 1H), 2.91 (dd, J = 7.8, 4.3 Hz, 1H), 2.47 (s, 3H), 2.28-2.22 (m, 1H) | 450.2 |
| 40 | 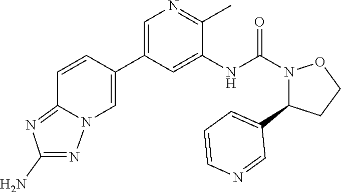 [SMILES: Cc1ncc(-c2ccc3nc(N)nn3c2)cc1NC(=O)N1OCC[C@H]1c1cccnc1] | (S)-N-(5-(2-amino-[1,2,4]triazolo[1,5- a]pyridin-6-yl)-2-methylpyridin-3-yl)-3- (pyridin-3-yl)isoxazolidine-2-carboxamide | 1H NMR (400 MHz, DMSO) 8 = 9.25 (s, 1H), 8.97 (d, J = 2.0 Hz, 1H), 8.68 (d, J = 2.5 Hz, 1H), 8.64 (s,, 1H), 8.51 (s, 1H), 8.09 (d, J = 2.5 Hz, 1H), 7.85- 7.75 (m, 2H), 7.49-7.40 (m, 2H), 6.11 (s, 2H), 5.48 (dd, J = 8.9, 5.6 Hz, 1H), 4.33 (q, J = 4.9 Hz, 1H), 4.04 (d, J = 9.8 Hz, 1H), 2.93 (dd, J = 7.6, 4.1 Hz, 1H), 2.47 (s, 3H), 2.32 (dq, J = 9.6, 6.4, 4.9 Hz, 1H) | 417.3 |
| 41 | 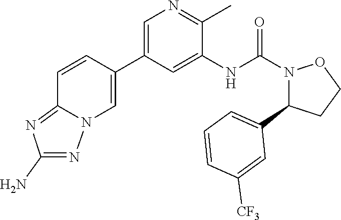 [SMILES: Cc1ncc(-c2ccc3nc(N)nn3c2)cc1NC(=O)N1OCC[C@H]1c1cccc(C(F)(F)F)c1] | (S)-N-(5-(2-amino-[1,2,4]triazolo[1,5- a]pyridin-6-yl)-2-methylpyridin-3-yl)-3-(3- (trifluoromethyl)phenyl)isoxazolidine-2- carboxamide | ¹H NMR (400 MHz, DMSO) δ = 9.26 (s, 1H), 8.97 (d, J = 2.0 Hz, 1H), 8.67 (d, J = 2.3 Hz, 1H), 8.09 (d, J = 2.3 Hz, 1H), 7.80-7.70 (m, 3H), 7.69-7.60 (m, 2H), 7.45 (d, J = 9.1 Hz, 1H), 6.10 (s, 2H), 5.54 (dd, J = 8.9, 5.9 Hz, 1H), 4.32 (td, J = 7.7, 2.8 Hz, 1H), 4.06-3.98 (m, 1H), 2.99-2.93 (m, 1H), 2.47 (s, 3H), 2.28 (td, J = 9.6, 8.8, 3.8 Hz, 1H) | 484.2 |
| 42 | 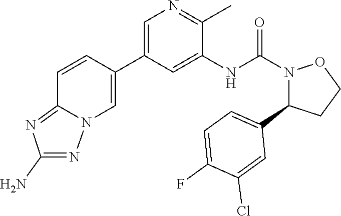 [SMILES: Cc1ncc(-c2ccc3nc(N)nn3c2)cc1NC(=O)N1OCC[C@H]1c1ccc(F)c(Cl)c1] | (S)-N-(5-(2-amino-[1,2,4]triazolo[1,5- a]pyridin-6-yl)-2-methylpyridin-3-yl)-3-(3- chloro-4-fluorophenyl)isoxazolidine-2- carboxamide | ¹H NMR (400 MHz, DMSO) δ = 9.23 (s, 1H), 8.97 (dd, J = 2.1, 0.8 Hz, 1H), 8.67 (d, J = 2.4 Hz, 1H), 8.08 (d, J = 2.3 Hz, 1H), 7.77 (dd, J = 9.2, 1.9 Hz, 1 H), 7.59 (d, J = 7.8 Hz, 1H), 7.48-7.38 (m, 3H), 6.10 (s, 2H), 5.43 (dd, J = 8.6, 5.9 Hz, 1H), 4.30 (td, J = 7.9, 3.0 Hz, 1H), 4.04-3.96 (m, 1H), 2.92-2.87 (m, 1H), 2.47 (s, 3H), 2.30-2.23 (m, 1H) | 468.2 |
| 43 | 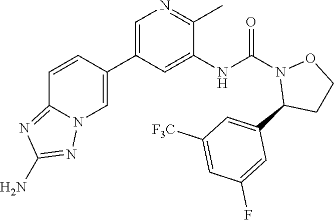 [SMILES: Cc1ncc(-c2ccc3nc(N)nn3c2)cc1NC(=O)N1OCC[C@H]1c1cc(F)cc(C(F)(F)F)c1] | (S)-N-(5-(2-amino-[1,2,4]triazolo[1,5- a]pyridin-6-yl)-2-methylpyridin-3-yl)-3-(3- fluoro-5- (trifluoromethyl)phenyl)isoxazolidine-2- carboxamide | ¹H NMR (400 MHz, DMSO-d₆) δ 9.30 (s, 1H), 8.97 (d, J = 1.8 Hz, 1H), 8.68 (d, J = 2.2 Hz, 1H), 8.09 (d, J = 2.3 Hz, 1H), 7.78 (dd, J = 9.1, 1.9 Hz, 1H), 7.64-7.55 (m, 3H), 7.45 (d, J = 9.1 Hz, 1H), 6.10 (s, 2H), 5.56 (dd, J = 8.7, 5.6 Hz, 1H), 4.32 (td, J = 8.0, 3.0 Hz, 1H), 4.02 (q, J = 8.1 Hz, 1H), 2.98-2.92 (m, 1H), 2.47 (s, 3H), 2.34-2.28 (m, 1H) | 502.2 |
| 44 | 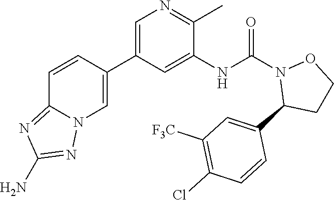 [SMILES: Cc1ncc(-c2ccc3nc(N)nn3c2)cc1NC(=O)N1OCC[C@H]1c1ccc(Cl)c(C(F)(F)F)c1] | (S)-N-(5-(2-amino-[1,2,4]triazolo[1,5- a]pyridin-6-yl)-2-methylpyridin-3-yl)-3-(4- chloro-3- (trifluoromethyl)phenyl)isoxazolidine-2- carboxamide | ¹H NMR (400 MHz, DMSO-d₆) δ 9.28 (s, 1H), 8.97 (d, J = 1.9 Hz, 1H), 8.68 (d, J = 2.2 Hz, 1H), 8.08 (d, J = 2.3 Hz, 1H), 7.85 (d, J = 1.7 Hz, 1H), 7.79-7.72 (m, 3H), 7.45 (d, J = 9.2 Hz, 1H), 6.10 (s, 2H), 5.53 (dd, J = 8.6, 5.7 Hz, 1H), 4.34-4.28 (m, 1H), 4.02 (q, J = 8.3 Hz, 1H), 2.97-2.91 (m, 1H), 2.47 (s, 3H), 2.29 (d, J = 6.0 Hz, 1H) | 518.2 |
| 45 | 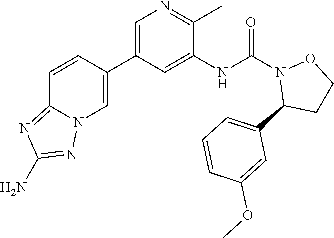 [SMILES: COc1cccc([C@@H]2CCON2C(=O)Nc2cc(-c3ccc4nc(N)nn4c3)cnc2C)c1] | (S)-N-(5-(2-amino-[1,2,4]triazolo[1,5- a]pyridin-6-yl)-2-methylpyridin-3-yl)-3-(3- methoxyphenyl)isoxazolidine-2- carboxamide | ¹H NMR (400 MHz, DMSO-d₆) δ 9.17 (s, 1H), 8.96 (t, J = 1.3 Hz, 1H), 8.66 (d, J = 2.3 Hz, 1H), 8.09 (d, J = 2.3 Hz, 1H), 7.77 (dd, J = 9.2, 1.9 Hz, 1H), 7.45 (d, J = 9.2 Hz, 1H), 7.28 (t, J = 7.9 Hz, 1H), 7.01-6.92 (m, 2H), 6.87- 6.81 (m, 1H), 6.10 (s, 2H), 5.39 (dd, J = 8.6, 5.7 Hz, 1H), 4.31-4.26 (m, 1H), 4.01 (d, J = 8.2 Hz, 1H), 3.76 (s, 3H), 2.90-2.84 (m, 1H), 2.47 (s, 3H), 2.26 (dd, J = 10.0, 6.3 Hz, 1H) | 446.3 |
| 46 | 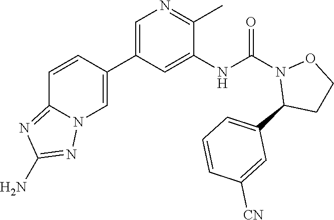 [SMILES: Cc1ncc(-c2ccc3nc(N)nn3c2)cc1NC(=O)N1OCC[C@H]1c1cccc(C#N)c1] | (S)-N-(5-(2-amino-[1,2,4]triazolo[1,5- a]pyridin-6-yl)-2-methylpyridin-3-yl)-3-(3- cyanophenyl)isoxazolidine-2-carboxamide | ¹H NMR (400 MHz, DMSO-d₆) δ 9.26 (s, 1H), 8.97 (s, 1H), 8.68 (d, J = 2.3 Hz, 1H), 8.08 (d, J = 2.2 Hz, 1H), 7.84 (s, 1H), 7.77 (d, J = 8.5 Hz, 3H), 7.60 (t, J = 7.8 Hz, 1H), 7.45 (d, J = 9.1 Hz, 1H), 6.10 (s, 2H), 5.51-5.46 (m, 1H), 4.31 (s, 1H), 4.02 (d, J = 8.3 Hz, 1H), 2.95-2.92 (m, 1H), 2.47 (s, 3H), 2.29 (s, 1H) | 441.2 |
| 47 | 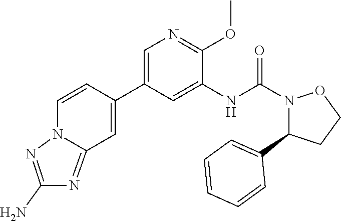 [SMILES: COc1ncc(-c2ccn3nc(N)nc3c2)cc1NC(=O)N1OCC[C@H]1c1ccccc1] | (S)-N-(5-(2-amino-[1,2,4]triazolo[1,5- a]pyridin-7-yl)-2-methoxypyridin-3-yl)-3- phenylisoxazolidine-2-carboxamide | ¹H NMR (400 MHz, DMSO-d₆) δ 8.65-8.54 (m, 2H), 8.51 (s, 1H), 8.35 (d, J = 2.3 Hz, 1H), 7.57 (d, J = 1.9 Hz, 1H), 7.44-7.32 (m, 4H), 7.31-7.25 (m, 1H), 7.13 (dd, J = 7.0, 2.0 Hz, 1H), 6.05 (s, 2H), 5.40 (dd, J = 8.5, 6.1 Hz, 1H), 4.32 (td, J = 7.8, 3.0 Hz, 1H), 4.03 (s, 3H), 4.00-3.94 (m, 1H), 2.94-2.87 (m, 1H), 2.30-2.23 (m, 1H) | 432.3 |
| 48 | 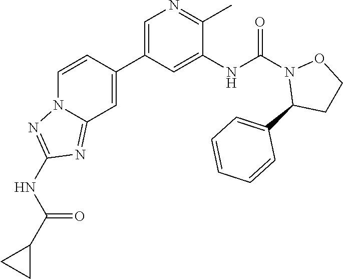 [SMILES: Cc1ncc(-c2ccn3nc(NC(=O)C4CC4)nc3c2)cc1NC(=O)N1OCC[C@H]1c1ccccc1] | (S)-N-(5-(2-(cyclopropanecarboxamido)- [1,2,4]triazolo[1,5-a]pyridin-7-yl)-2- methylpyridin-3-yl)-3-phenylisoxazolidine- 2-carboxamide | ¹H NMR (400 MHz, DMSO) δ = 11.10 (s, 1H), 9.22 (s, 1H), 8.91 (d, J = 7.3 Hz, 1H), 8.81 (s, 1H), 8.24 (s, 1H), 8.05 (s, 1H), 7.54-7.23 (m, 6H), 5.47-5.36 (m, 1H), 4.31 (m, 1H), 4.02 (m, 1H), 2.90 (m, 2H), 2.30 (d, J = 25.0 Hz, 2H), 2.06 (m, 2H), 1.24 (m, 1H), 0.83 (s, 3H) | 484.2 |
| 49 | 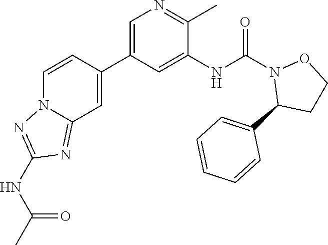 [SMILES: CC(=O)Nc1nc2cc(-c3cnc(C)c(NC(=O)N4OCC[C@H]4c4ccccc4)c3)ccn2n1] | (S)-N-(5-(2-acetamido-[1,2,4]triazolo[1,5- a]pyridin-7-yl)-2-methylpyridin-3-yl)-3- phenylisoxazolidine-2-carboxamide | ¹H NMR (400 MHz, DMSO) δ = 10.83 (s, 1H), 9.23 (s, 1H), 8.92 (d, J = 7.3 Hz, 1H), 8.80 (s, 1H), 8.24 (s, 1H), 8.04 (s, 1H), 7.57-7.18 (m, 6H), 5.43 (m, 1H), 4.31 (m, 1H), 4.08-3.95 (m, 1H), 3.21 (s, 3H), 2.91 (m, 1H), 2.27 (m, 1H), 2.16 (s, 3H) | 458.2 |
| 50 | 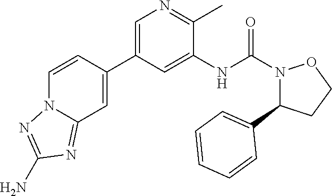 [SMILES: Cc1ncc(-c2ccn3nc(N)nc3c2)cc1NC(=O)N1OCC[C@H]1c1ccccc1] | (S)-N-(5-(2-amino-[1,2,4]triazolo[1,5- a]pyridin-7-yl)-2-methylpyridin-3-yl)-3- phenylisoxazolidine-2-carboxamide | ¹H NMR (400 MHz, DMSO) δ = 9.19 (s, 1H), 8.74 (s, 1H), 8.62 (d, J = 6.9 Hz, 1H), 8.17 (s, 1H), 7.70 (s, 1H), 7.38 (dd, J = 13.0, 5.9 Hz, 3H), 7.32-7.19 (m, 2H), 6.08 (s, 2H), 5.41 (d, J = 7.2 Hz, 1H), 4.30 (s, 1H), 4.00 (t, J = 8.3 Hz, 1H), 2.93-2.86 (m, 1H), 2.30-2.24 (m, 1H), 1.24 (s, 3H), 0.85 (d, J = 8.0 Hz, 1H) | 416.3 |
| 51 | 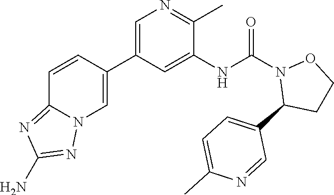 [SMILES: Cc1ccc([C@@H]2CCON2C(=O)Nc2cc(-c3ccc4nc(N)nn4c3)cnc2C)cn1] | (S)-N-(5-(2-amino-[1,2,4]triazolo[1,5- a]pyridin-6-yl)-2-methylpyridin-3-yl)-3-(6- methylpyridin-3-yl)isoxazolidine-2- carboxamide | ¹H NMR (400 MHz, DMSO-d₆) δ = 9.21 (s, 1H), 8.97 (d, J = 1.8 Hz, 1H), 8.67 (d, J = 2.2 Hz, 1H), 8.47 (d, J = 2.3 Hz, 1H), 8.08 (d, J = 2.2 Hz, 1H), 7.77 (dd, J = 9.1, 1.9 Hz, 1H), 7.68 (dd, J = 8.1, 2.4 Hz, 1H), 7.45 (d, J = 9.1 Hz, 1H), 7.26 (d, J = 8.0 Hz, 1H), 6.10 (s, 2H), 5.44 (dd, J = 8.5, 5.6 Hz, 1H), 4.31 (td, J = 7.9, 3.2 Hz, 1H), 4.03 (q, J = 8.1 Hz, 1H), 2.88 (dtd, J = 11.9, 7.8, 3.2 Hz, 1H), 2.46 (d, J = 1.7 Hz, 6H), 2.29 (dq, J = 14.0, 8.2 Hz, 1H) | 431.2 |
| 52 | 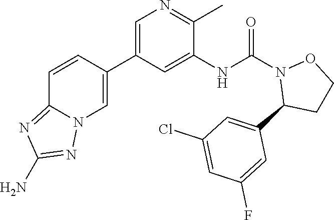 [SMILES: Cc1ncc(-c2ccc3nc(N)nn3c2)cc1NC(=O)N1OCC[C@H]1c1cc(F)cc(Cl)c1] | (S)-N-(5-(2-amino-[1,2,4]triazolo[1,5- a]pyridin-6-yl)-2-methylpyridin-3-yl)-3-(3- chloro-5-fluorophenyl)isoxazolidine-2- carboxamide | ¹H NMR (400 MHz, DMSO-d₆) δ = 9.28 (s, 1H), 8.99 (d, J = 1.8 Hz, 1H), 8.70 (d, J = 2.2 Hz, 1H), 8.12 (d, J = 2.2 Hz, 1H), 7.79 (dd, J = 9.2, 1.9 Hz, 1H), 7.46 (d, J = 9.1 Hz, 1H), 7.38-7.31 (m, 2H), 7.24 (dt, J = 9.8, 1.9 Hz, 1H), 6.14 (s, 2H), 5.46 (dd, J = 8.7, 5.7 Hz, 1H), 4.30 (td, J = 7.9, 2.9 Hz, 1H), 4.00 (dt, J = 9.4, 7.5 Hz, 1H), 2.92 (tdt, J = 9.6, 7.2, 3.0 Hz, 1H), 2.48 (s, 3H), 2.27 (dtd, J = 12.3, 8.0, 5.7 Hz, 1H) | 468.2 |
| 53 | 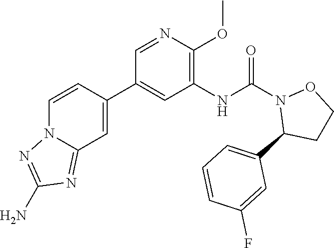 [SMILES: COc1ncc(-c2ccn3nc(N)nc3c2)cc1NC(=O)N1OCC[C@H]1c1cccc(F)c1] | (S)-N-(5-(2-amino-[1,2,4]triazolo[1,5- a]pyridin-7-yl)-2-methoxypyridin-3-yl)-3- (3-fluorophenyl)isoxazolidine-2- carboxamide | ¹H NMR (400 MHz, DMSO-d₆) δ = 8.62-8.58 (m, 2H), 8.55 (s, 1H), 8.37 (d, J = 2.3 Hz, 1H), 7.58 (dd, J = 2.0, 0.9 Hz, 1H), 7.43 (td, J = 8.0, 6.1 Hz, 1H), 7.25 (d, J = 7.7 Hz, 1H), 7.21 (dt, J = 10.2, 2.2 Hz, 1H), 7.17-7.09 (m, 2H), 6.08 (s, 2H), 5.43 (dd, J = 8.5, 6.2 Hz, 1H), 4.32 (td, J = 7.8, 3.0 Hz, 1H), 4.03 (s, 3H), 4.01-3.96 (m, 1H), 2.92 (dddd, J = 12.1, 9.4, 6.8, 3.0 Hz, 1H), 2.32-2.22 (m, 1H) | 450.1 |
| 54 | 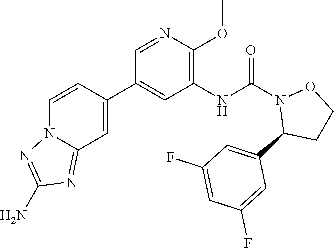 [SMILES: COc1ncc(-c2ccn3nc(N)nc3c2)cc1NC(=O)N1OCC[C@H]1c1cc(F)cc(F)c1] | (S)-N-(5-(2-amino-[1,2,4]triazolo[1,5- a]pyridin-7-yl)-2-methoxypyridin-3-yl)-3- (3,5-difluorophenyl)isoxazolidine-2- carboxamide | ¹H NMR (400 MHz, DMSO-d₆) δ = 8.62-8.56 (m, 3H), 8.38 (d, J = 2.3 Hz, 1H), 7.59 (dd, J = 2.0, 0.9 Hz, 1H), 7.22- 7.07 (m, 4H), 6.08 (s, 2H), 5.44 (dd, J = 8.5, 6.1 Hz, 1H), 4.31 (td, J = 7.9, 2.9 Hz, 1H), 4.03 (s, 3H), 4.01-3.95 (m, 1H), 2.93 (tdd, J = 12.0, 6.7, 3.0 Hz, 1H), 2.27 (t, J = 10.3 Hz, 1H) | 468.1 |
| 55 | 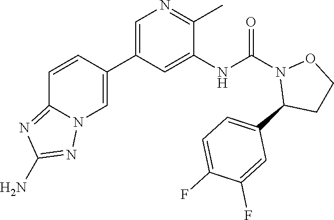 [SMILES: Cc1ncc(-c2ccc3nc(N)nn3c2)cc1NC(=O)N1OCC[C@H]1c1ccc(F)c(F)c1] | (S)-N-(5-(2-amino-[1,2,4]triazolo[1,5- a]pyridin-6-yl)-2-methylpyridin-3-yl)-3- (3,4-difluorophenyl)isoxazolidine-2- carboxamide | ¹H NMR (400 MHz, DMSO-d₆) δ = 9.23 (s, 1H), 8.97 (d, J = 1.9 Hz, 1H), 8.68 (t, J = 2.9 Hz, 1H), 8.08 (d, J = 2.3 Hz, 1H), 7.78 (dt, J = 9.2, 2.9 Hz, 1H), 7.49-7.39 (m, 3H), 7.30-7.23 (m, 1H), 6.10 (s, 2H), 5.43 (dd, J = 8.7, 5.4 Hz, 1H), 4.29 (dt, J = 8.0, 4.1 Hz, 1H), 4.00 (q, J = 8.0 Hz, 1H), 2.89 (dtd, J = 11.8, 7.8, 3.0 Hz, 1H), 2.47 (s, 3H), 2.27 (ddd, J = 14.0, 8.9, 6.0 Hz, 1H) | 452.2 |
| 56 | 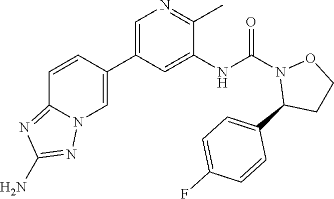 [SMILES: Cc1ncc(-c2ccc3nc(N)nn3c2)cc1NC(=O)N1OCC[C@H]1c1ccc(F)cc1] | (S)-N-(5-(2-amino-[1,2,4]triazolo[1,5- a]pyridin-6-yl)-2-methylpyridin-3-yl)-3-(4- fluorophenyl)isoxazolidine-2-carboxamide | ¹H NMR (400 MHz, DMSO-d₆) δ = 9.19 (s, 1H), 8.97 (d, J = 1.8 Hz, 1H), 8.67 (d, J = 2.2 Hz, 1H), 8.08 (d, J = 2.2 Hz, 1H), 7.77 (dd, J = 9.2, 1.9 Hz, 1H), 7.48-7.40 (m, 3H), 7.19 (t, J = 8.8 Hz, 2H), 6.10 (s, 2H), 5.42 (dd, J = 8.6, 5.8 Hz, 1H), 4.30 (td, J = 7.9, 3.0 Hz, 1H), 4.00 (q, J = 8.1 Hz, 1H), 2.88 (dtd, J = 11.5, 8.0, 3.1 Hz, 1H), 2.47 (s, 3H), 2.29- 2.21 (m, 1H) | 434.1 |
| 57 | 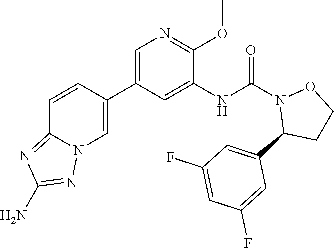 [SMILES: COc1ncc(-c2ccc3nc(N)nn3c2)cc1NC(=O)N1OCC[C@H]1c1cc(F)cc(F)c1] | (S)-N-(5-(2-amino-[1,2,4]triazolo[1,5- a]pyridin-6-yl)-2-methoxypyridin-3-yl)-3- (3,5-difluorophenyl)isoxazolidine-2- carboxamide | ¹H NMR (400 MHz, DMSO-d₆) = 8.84 (d, J = 1.9 Hz, 1H), 8.59-8.48 (m, 2H), 8.26 (d, J = 2.3 Hz, 1H), 7.67 (dd, J = 9.2, 1.9 Hz, 1H), 7.44 (d, J = 9.1 Hz, 1H), 7.15 (ddd, J = 12.6, 9.0, 6.8 Hz, 3H), 6.08 (s, 2H), 5.43 (dd, J = 8.6, 6.1 Hz, 1H), 4.31 (dt, J = 8.0, 4.0 Hz, 1H), 4.01 (s, 4H), 2.98- 2.87 (m, 1H), 2.36-2.22 (m, 1H) | 468.2 |
| 58 | 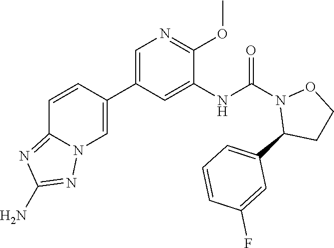 [SMILES: COc1ncc(-c2ccc3nc(N)nn3c2)cc1NC(=O)N1OCC[C@H]1c1cccc(F)c1] | (S)-N-(5-(2-amino-[1,2,4]triazolo[1,5- a]pyridin-6-yl)-2-methoxypyridin-3-yl)-3- (3-fluorophenyl)isoxazolidine-2- carboxamide | ¹H NMR (400 MHz, DMSO-d₆) δ = 8.84 (dd, J = 1.9, 0.9 Hz, 1H), 8.56-8.49 (m, 2H), 8.25 (d, J = 2.3 Hz, 1H), 7.67 (dd, J = 9.1, 1.9 Hz, 1H), 7.46-7.39 (m, 2H), 7.27-7.17 (m, 2H), 7.11 (td, J = 8.6, 2.7 Hz, 1H), 6.07 (s, 2H), 5.42 (dd, J = 8.5, 6.1 Hz, 1H), 4.32 (td, J = 7.8, 3.0 Hz, 1H), 4.02 (s, 4H), 2.96-2.87 (m, 1H), 2.32-2.22 (m, 1H) | 450.2 |
| 59 | 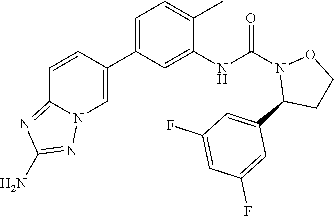 [SMILES: Cc1ccc(-c2ccc3nc(N)nn3c2)cc1NC(=O)N1OCC[C@H]1c1cc(F)cc(F)c1] | (S)-N-(5-(2-amino-[1,2,4]triazolo[1,5- a]pyridin-6-yl)-2-methylphenyl)-3-(3,5- difluorophenyl)isoxazolidine-2- carboxamide | ¹H NMR (400 MHz, DMSO-d₆) δ = 9.05 (s, 1H), 8.82 (d, J = 1.9 Hz, 1H), 7.72 (dd, J = 6.8, 2.1 Hz, 2H), 7.49 (dd, J = 7.9, 2.0 Hz, 1H), 7.42 (d, J = 9.1 Hz, 1H), 7.32 (d, J = 7.9 Hz, 1H), 7.16-7.08 (m, 3H), 6.05 (s, 2H), 5.46 (dd, J = 8.8, 5.6 Hz, 1H), 4.27 (td, J = 8.0, 3.1 Hz, 1H), 4.00 (q, J = 8.1 Hz, 1H), 2.89 (dtd, J = 11.8, 8.2, 3.0 Hz, 1H), 2.26 (s, 4H) | 451.2 |
| 60 | 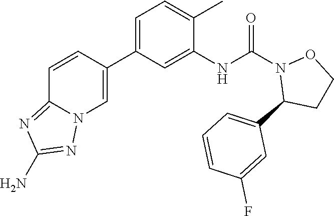 [SMILES: Cc1ccc(-c2ccc3nc(N)nn3c2)cc1NC(=O)N1OCC[C@H]1c1cccc(F)c1] | (S)-N-(5-(2-amino-[1,2,4]triazolo[1,5- a]pyridin-6-yl)-2-methylphenyl)-3-(3- fluorophenyl)isoxazolidine-2-carboxamide | ¹H NMR (400 MHz, DMSO-d₆) δ = 9.07-9.02 (m, 2H), 8.02 (dd, J = 9.1, 1.8 Hz, 1H), 7.79 (d, J = 2.0 Hz, 1H), 7.63 (d, J = 9.1 Hz, 1H), 7.53 (dd, J = 7.9, 2.0 Hz, 1H), 7.42 (td, J = 8.0, 6.1 Hz, 1H), 7.36 (d, J = 8.1 Hz, 1H), 7.25 (d, J = 7.7 Hz, 1H), 7.21 (dt, J = 10.3, 2.2 Hz, 1H), 7.11 (td, J = 8.7, 2.7 Hz, 1H), 5.44 (dd, J = 8.6, 5.6 Hz, 1H), 4.28 (td, J = 8.0, 3.2 Hz, 1H), 3.99 (dd, J = 7.9, 1.7 Hz, 1H), 2.89 (dddd, J = 12.1, 8.7, 7.2, 3.2 Hz, 1H), 2.32-2.21 (m, 4H) | 433.2 |
| 61 | 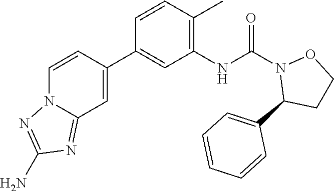 [SMILES: Cc1ccc(-c2ccn3nc(N)nc3c2)cc1NC(=O)N1OCC[C@H]1c1ccccc1] | (S)-N-(5-(2-amino-[1,2,4]triazolo[1,5- a]pyridin-7-yl)-2-methylphenyl)-3- phenylisoxazolidine-2-carboxamide | ¹H NMR (400 MHz, DMSO-d₆) δ = 9.01 (s, 1H), 8.57 (dd, J = 6.9, 0.8 Hz, 1H), 7.83 (d, J = 2.0 Hz, 1H), 7.58 (dd, J = 2.1, 0.9 Hz, 1H), 7.57-7.53 (m, 1H), 7.44-7.33 (m, 5H), 7.31-7.24 (m, 1H), 7.16 (dd, J = 7.1, 2.0 Hz, 1H), 6.05 (s, 2H), 5.42 (dd, J = 8.6, 5.7 Hz, 1H), 4.28 (td, J = 7.9, 3.1 Hz, 1H), 4.00 (dt, J = 9.1, 7.4 Hz, 1H), 2.89 (tdd, J = 12.0, 7.2, 3.1 Hz, 1H), 2.28 (s, 3H), 2.24 (ddd, J = 9.4, 7.9, 4.8 Hz, 1H) | 415.1 |
| 62 | 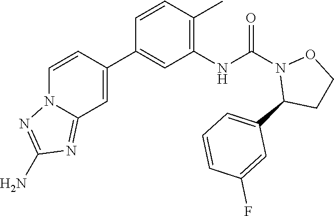 [SMILES: Cc1ccc(-c2ccn3nc(N)nc3c2)cc1NC(=O)N1OCC[C@H]1c1cccc(F)c1] | (S)-N-(5-(2-amino-[1,2,4]triazolo[1,5- a]pyridin-7-yl)-2-methylphenyl)-3-(3- fluorophenyl)isoxazolidine-2-carboxamide | ¹H NMR (400 MHz, DMSO-d₆) δ = 9.05 (s, 1H), 8.57 (d, J = 7.0 Hz, 1H), 7.82 (d, J = 2.0 Hz, 1H), 7.60-7.53 (m, 2H), 7.43 (td, J = 8.0, 6.1 Hz, 1H), 7.36 (d, J = 8.0 Hz, 1H), 7.26 (d, J = 7.8 Hz, 1H), 7.21 (dt, J = 10.3, 2.2 Hz, 1H), 7.16 (dd, J = 7.0, 2.0 Hz, 1H), 7.11 (td, J = 8.7, 2.7 Hz, 1H), 6.05 (s, 2H), 5.45 (dd, J = 8.6, 5.6 Hz, 1H), 4.28 (td, J = 8.0, 3.1 Hz, 1H), 4.01 (dt, J = 9.2, 7.5 Hz, 1H), 2.90 (dtd, J = 11.6, 8.1, 3.1 Hz, 1H), 2.28 (s, 3H), 2.27-2.21 (m, 1H) | 433.1 |
| 63 | 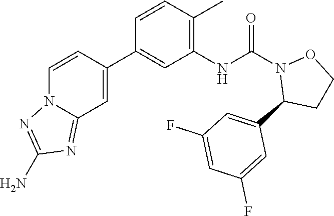 [SMILES: Cc1ccc(-c2ccn3nc(N)nc3c2)cc1NC(=O)N1OCC[C@H]1c1cc(F)cc(F)c1] | (S)-N-(5-(2-amino-[1,2,4]triazolo[1,5- a]pyridin-7-yl)-2-methylphenyl)-3-(3,5- difluorophenyl)isoxazolidine-2- carboxamide | ¹H NMR (400 MHz, DMSO-d₆) δ = 9.09 (s, 1H), 8.57 (dd, J = 7.0, 0.8 Hz, 1H), 7.81 (d, J = 2.0 Hz, 1H), 7.60-7.55 (m, 2H), 7.36 (d, J = 8.1 Hz, 1H), 7.19-7.09 (m, 4H), 6.05 (s, 2H), 5.47 (dd, J = 8.7, 5.6 Hz, 1H), 4.28 (td, J = 8.0, 3.1 Hz, 1H), 4.00 (dt, J = 9.2, 7.5 Hz, 1H), 2.90 (dddd, J = 12.1, 8.8, 7.1, 3.1 Hz, 1H), 2.28 (s, 3H), 2.27-2.19 (m, 1H) | 451.1 |
| 64 | 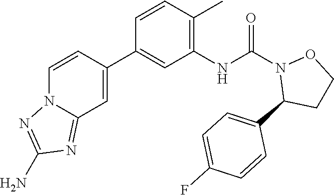 [SMILES: Cc1ccc(-c2ccn3nc(N)nc3c2)cc1NC(=O)N1OCC[C@H]1c1ccc(F)cc1] | (S)-N-(5-(2-amino-[1,2,4]triazolo[1,5- a]pyridin-7-yl)-2-methylphenyl)-3-(4- fluorophenyl)isoxazolidine-2-carboxamide | ¹H NMR (400 MHz, DMSO-d₆) δ = 9.02 (s, 1H), 8.57 (dd, J = 7.1, 0.8 Hz, 1H), 7.82 (d, J = 2.0 Hz, 1H), 7.59-7.54 (m, 2H), 7.47-7.42 (m, 2H), 7.35 (d, J = 8.0 Hz, 1H), 7.23- 7.17 (m, 2H), 7.16 (dd, J = 7.0, 2.0 Hz, 1H), 6.05 (s, 2H), 5.43 (dd, J = 8.6, 5.7 Hz, 1H), 4.28 (td, J = 8.0, 3.1 Hz, 1H), 4.00 (dt, J = 9.1, 7.4 Hz, 1H), 2.94-2.82 (m, 1H), 2.28 (s, 3H), 2.26-2.20 (m, 1H) | 433.1 |
| 65 | 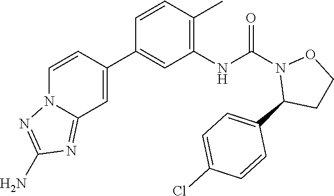 [SMILES: Cc1ccc(-c2ccn3nc(N)nc3c2)cc1NC(=O)N1OCC[C@H]1c1ccc(Cl)cc1] | (S)-N-(5-(2-amino-[1,2,4]triazolo[1,5- a]pyridin-7-yl)-2-methylphenyl)-3-(4- chlorophenyl)isoxazolidine-2-carboxamide | ¹H NMR (400 MHz, DMSO-d₆) δ = 9.04 (s, 1H), 8.57 (dd, J = 7.0, 0.8 Hz, 1H), 7.81 (d, J = 2.0 Hz, 1H), 7.60-7.54 (m, 2H), 7.44 (s, 4H), 7.35 (d, J = 8.1 Hz, 1H), 7.16 (dd, J = 7.0, 2.0 Hz, 1H), 6.05 (s, 2H), 5.43 (dd, J = 8.6, 5.6 Hz, 1H), 4.28 (td, J = 8.0, 3.1 Hz, 1H), 4.00 (dt, J = 9.2, 7.4 Hz, 1H), 2.95-2.84 (m, 1H), 2.28 (s, 3H), 2.26-2.17 (m, 1H) | 449.0 |
| 66 | 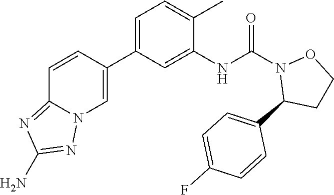 [SMILES: Cc1ccc(-c2ccc3nc(N)nn3c2)cc1NC(=O)N1OCC[C@H]1c1ccc(F)cc1] | (S)-N-(5-(2-amino-[1,2,4]triazolo[1,5- a]pyridin-6-yl)-2-methylphenyl)-3-(4- fluorophenyl)isoxazolidine-2-carboxamide | ¹H NMR (400 MHz, DMSO-d₆) δ = 9.01-8.96 (m, 2H), 7.93 (dd, J = 9.2, 1.9 Hz, 1H), 7.78 (d, J = 2.1 Hz, 1H), 7.56 (d, J = 9.1 Hz, 1H), 7.51 (dd, J = 7.9, 2.1 Hz, 1H), 7.43 (qd, J = 6.9, 6.2, 1.7 Hz, 3H), 7.35 (d, J = 8.4 Hz, 1H), 7.19 (td, J = 8.9, 2.9 Hz, 3H), 5.45-5.39 (m, 1H), 4.28 (td, J = 7.8, 3.3 Hz, 1H), 4.03-3.96 (m, 1H), 2.87 (dddd, J = 12.1, 8.8, 7.2, 3.3 Hz, 1H), 2.27 (s, 3H), 2.24-2.19 (m, 1H) | 433.2 |
| 67 | 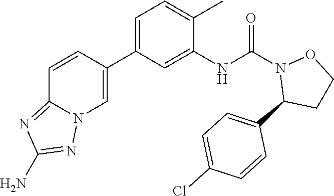 [SMILES: Cc1ccc(-c2ccc3nc(N)nn3c2)cc1NC(=O)N1OCC[C@H]1c1ccc(Cl)cc1] | (S)-N-(5-(2-amino-[1,2,4]triazolo[1,5- a]pyridin-6-yl)-2-methylphenyl)-3-(4- chlorophenyl)isoxazolidine-2-carboxamide | ¹H NMR (400 MHz, DMSO-d₆) δ = 9.03-8.97 (m, 2H), 7.93 (dd, J = 9.1, 1.8 Hz, 1H), 7.77 (d, J = 2.0 Hz, 1H), 7.56 (d, J = 9.1 Hz, 1H), 7.51 (dd, J = 7.9, 2.0 Hz, 1H), 7.42 (d, J = 4.9 Hz, 5H), 7.35 (d, J = 8.0 Hz, 1H), 5.42 (dd, J = 8.5, 5.7 Hz, 1H), 4.28 (td, J = 8.0, 3.2 Hz, 1H), 4.02-3.97 (m, 1H), 2.88 (dtd, J = 12.1, 8.4, 3.2 Hz, 1H), 2.30-2.17 (m, 5H) | 449.2 |
| 68 | 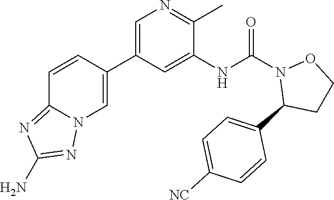 [SMILES: Cc1ncc(-c2ccc3nc(N)nn3c2)cc1NC(=O)N1OCC[C@H]1c1ccc(C#N)cc1] | (S)-N-(5-(2-amino-[1,2,4]triazolo[1,5- a]pyridin-6-yl)-2-methylpyridin-3-yl)-3-(4- cyanophenyl)isoxazolidine-2-carboxamide | ¹H NMR (400 MHz, DMSO-d₆) δ = 9.28 (s, 1H), 8.98 (d, J = 1.8 Hz, 1H), 8.69 (d, J = 2.2 Hz, 1H), 8.10 (d, J = 2.3 Hz, 1H), 7.88-7.82 (m, 2H), 7.78 (dd, J = 9.2, 1.9 Hz, 1H), 7.63-7.58 (m, 2H), 7.46 (d, J = 9.1 Hz, 1H), 6.12 (s, 2H), 5.50 (dd, J = 8.7, 5.9 Hz, 1H), 4.31 (td, J = 8.0, 2.9 Hz, 1H), 4.01 (dt, J = 9.4, 7.3 Hz, 1H), 2.95 (tdd, J = 9.4, 7.2, 2.9 Hz, 1H), 2.47 (s, 3H), 2.28-2.20 (m, 1H) | 441.2 |
| 69 | 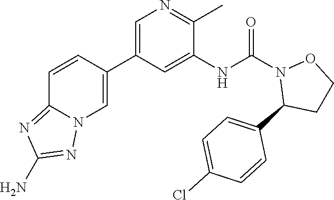 [SMILES: Cc1ncc(-c2ccc3nc(N)nn3c2)cc1NC(=O)N1OCC[C@H]1c1ccc(Cl)cc1] | (S)-N-(5-(2-amino-[1,2,4]triazolo[1,5- a]pyridin-6-yl)-2-methylpyridin-3-yl)-3-(4- chlorophenyl)isoxazolidine-2-carboxamide | ¹H NMR (400 MHz, DMSO-d₆) δ = 9.24 (s, 1H), 8.99 (d, J = 1.8 Hz, 1H), 8.69 (d, J = 2.2 Hz, 1H), 8.13 (d, J = 2.3 Hz, 1H), 7.79 (dd, J = 9.2, 1.9 Hz, 1H), 7.47 (d, J = 9.2 Hz, 1H), 7.43 (s, 4H), 6.15 (s, 2H), 5.42 (dd, J = 8.6, 5.8 Hz, 1H), 4.30 (td, J = 7.9, 3.0 Hz, 1H), 4.00 (dt, J = 9.1, 7.4 Hz, 1H), 2.93-2.85 (m, 1H), 2.48 (s, 3H), 2.24 (dddd, J = 12.1, 9.3, 7.8, 5.7 Hz, 1H) | 450.1 |
| 70 | 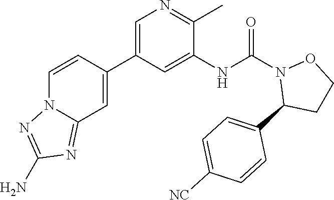 [SMILES: Cc1ncc(-c2ccn3nc(N)nc3c2)cc1NC(=O)N1OCC[C@H]1c1ccc(C#N)cc1] | (S)-N-(5-(2-amino-[1,2,4]triazolo[1,5- a]pyridin-7-yl)-2-methylpyridin-3-yl)-3-(4- cyanophenyl)isoxazolidine-2-carboxamide | ¹H NMR (400 MHz, DMSO-d₆) δ = 9.27 (s, 1H), 8.75 (d, J = 2.2 Hz, 1H), 8.61 (d, J = 7.0 Hz, 1H), 8.15 (d, J = 2.2 Hz, 1H), 7.91-7.79 (m, 2H), 7.70 (d, J = 2.0 Hz, 1H), 7.61 (dd, J = 8.3, 1.8 Hz, 2H), 7.22 (dd, J = 7.0, 2.0 Hz, 1H), 6.07 (s, 2H), 5.51 (dd, J = 8.7, 5.9 Hz, 1H), 4.36-4.25 (m, 1H), 4.02 (q, J = 8.1 Hz, 1H), 3.01-2.90 (m, 1H), 2.49 (s, 3H), 2.30-2.19 (m, 1H) | 441.2 |
| 71 | 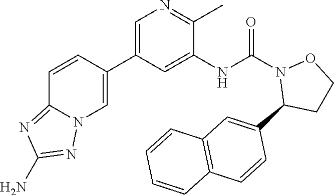 [SMILES: Cc1ncc(-c2ccc3nc(N)nn3c2)cc1NC(=O)N1OCC[C@H]1c1ccc2ccccc2c1] | (S)-N-(5-(2-amino-[1,2,4]triazolo[1,5- a]pyridin-6-yl)-2-methylpyridin-3-yl)-3- (naphthalen-2-yl)isoxazolidine-2- carboxamide | ¹H NMR (400 MHz, DMSO-d₆) δ = 9.22 (s, 1H), 8.97 (d, J = 1.8 Hz, 1H), 8.67 (d, J = 2.2 Hz, 1H), 8.11 (d, J= 2.2 Hz, 1H), 7.93 (ddt, J = 8.8, 5.2, 3.1 Hz, 4H), 7.77 (dd, J = 9.2, 1.9 Hz, 1H), 7.57 (dd, J = 8.5, 1.8 Hz, 1H), 7.54-7.48 (m, 2H), 7.45 (d, J = 9.1 Hz, 1H), 6.10 (s, 2H), 5.59 (dd, J = 8.6, 5.8 Hz, 1H), 4.36 (td, J = 7.9, 2.9 Hz, 1H), 4.06 (dt, J = 9.2, 7.4 Hz, 1H), 2.98 (ddt, J = 12.7, 9.1, 4.3 Hz, 1H), 2.49 (s, 3H), 2.41-2.32 (m, 1H) | 466.2 |
| 72 | 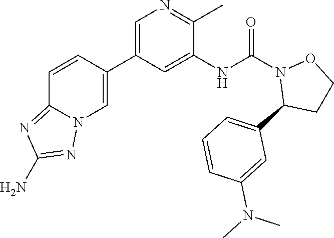 [SMILES: Cc1ncc(-c2ccc3nc(N)nn3c2)cc1NC(=O)N1OCC[C@H]1c1cccc(N(C)C)c1] | (S)-N-(5-(2-amino-[1,2,4]triazolo[1,5- a]pyridin-6-yl)-2-methylpyridin-3-yl)-3-(3- (dimethylamino)phenyl)isoxazolidine-2- carboxamide | ¹H NMR (400 MHz, DMSO-d₆) δ = 9.14 (s, 1H), 8.97- 8.94 (m, 1H), 8.66 (d, J = 2.2 Hz, 1H), 8.09 (d, J = 2.3 Hz, 1H), 7.77 (dd, J = 9.2, 1.9 Hz, 1H), 7.45 (d, J = 9.1 Hz, 1H), 7.16 (t, J = 7.9 Hz, 1H), 6.73 (t, J = 2.1 Hz, 1H), 6.69 (d, J = 7.5 Hz, 1H), 6.62 (dd, J = 8.1, 2.6 Hz, 1H), 6.10 (s, 2H), 5.34 (dd, J = 8.6, 5.6 Hz, 1H), 4.28 (td, J = 7.9, 3.2 Hz, 1H), 3.98 (dt, J = 9.3, 7.5 Hz, 1H), 2.90 (s, 7H), 2.47 (s, 3H), 2.29-2.20 (m, 1H) | 459.2 |
| 73 | 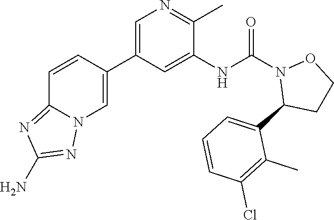 [SMILES: Cc1ncc(-c2ccc3nc(N)nn3c2)cc1NC(=O)N1OCC[C@H]1c1cccc(Cl)c1C] | (S)-N-(5-(2-amino-[1,2,4]triazolo[1,5- a]pyridin-6-yl)-2-methylpyridin-3-yl)-3-(3- chloro-2-methylphenyl)isoxazolidine-2- carboxamide | ¹H NMR (400 MHz, DMSO-d₆) δ = 9.23 (s, 1H), 8.96 (dd, J = 2.0, 0.9 Hz, 1H), 8.67 (d, J = 2.2 Hz, 1H), 8.08 (d, J = 2.2 Hz, 1H), 7.77 (dd, J = 9.2, 1.9 Hz, 1H), 7.50-7.39 (m, 2H), 7.37 (dd, J = 8.0, 1.4 Hz, 1H), 7.25 (t, J = 7.8 Hz, 1H), 6.10 (s, 2H), 5.60 (dd, J = 8.7, 5.6 Hz, 1H), 4.32 (td, J = 8.0, 3.0 Hz, 1H), 4.03 (q, J = 8.0 Hz, 1H), 3.02-2.94 (m, 1H), 2.47 (s, 3H), 2.40 (s, 3H), 2.15-2.05 (m, 1H) | 464.2 |
| 74 | 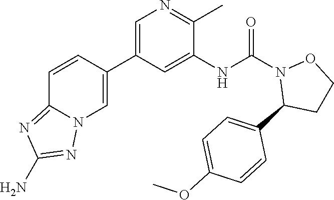 [SMILES: COc1ccc([C@@H]2CCON2C(=O)Nc2cc(-c3ccc4nc(N)nn4c3)cnc2C)cc1] | (S)-N-(5-(2-amino-[1,2,4]triazolo[1,5- a]pyridin-6-yl)-2-methylpyridin-3-yl)-3-(4- methoxyphenyl)isoxazolidine-2- carboxamide | ¹H NMR (400 MHz, DMSO-d₆) δ = 9.16 (s, 1H), 8.98 (d, J = 1.8 Hz, 1H), 8.68 (d, J = 2.2 Hz, 1H), 8.12 (d, J = 2.3 Hz, 1H), 7.79 (dd, J = 9.2, 1.9 Hz, 1H), 7.46 (d, J = 9.1 Hz, 1H), 7.34-7.28 (m, 2H), 6.95-6.90 (m, 2H), 6.14 (s, 2H), 5.36 (dd, J = 8.5, 5.6 Hz, 1H), 4.29 (td, J = 8.0, 3.3 Hz, 1H), 4.00 (dt, J = 9.3, 7.5 Hz, 1H), 3.75 (s, 3H), 2.89-2.78 (m, 1H), 2.47 (s, 3H), 2.31-2.20 (m, 1H) | 446.2 |
| 75 | 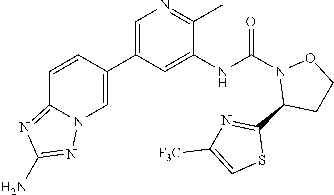 [SMILES: Cc1ncc(-c2ccc3nc(N)nn3c2)cc1NC(=O)N1OCC[C@H]1c1nc(C(F)(F)F)cs1] | (S)-N-(5-(2-amino-[1,2,4]triazolo[1,5- a]pyridin-6-yl)-2-methylpyridin-3-yl)-3-(4- (trifluoromethyl)thiazole-2-yl)isoxazolidine- 2-carboxamide | ¹H NMR (400 MHz, DMSO-d₆) δ = 9.46 (s, 1H), 8.99 (d, J = 1.8 Hz, 1H), 8.71 (d, J = 2.2 Hz, 1H), 8.48 (d, J = 1.1 Hz, 1H), 8.09 (d, J = 2.2 Hz, 1H), 7.79 (dd, J = 9.1, 1.9 Hz, 1H), 7.49-7.42 (m, 1H), 6.11 (s, 2H), 5.75 (dd, J = 8.8, 5.0 Hz, 1H), 4.35 (td, J = 8.1, 3.2 Hz, 1H), 4.06 (q, J = 8.2 Hz, 1H), 3.01-2.91 (m, 1H), 2.66 (dtd, J = 13.1, 8.6, 5.0 Hz, 1H), 2.48 (s, 3H) | 491.1 |
| 76 | 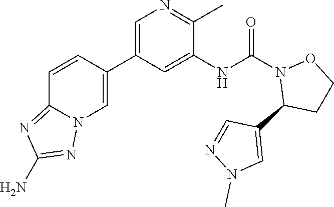 [SMILES: Cc1ncc(-c2ccc3nc(N)nn3c2)cc1NC(=O)N1OCC[C@H]1c1cnn(C)c1] | (S)-N-(5-(2-amino-[1,2,4]triazolo[1,5- a]pyridin-6-yl)-2-methylpyridin-3-yl)-3-(1- methyl-1H-pyrazol-4-yl)isoxazolidine-2- carboxamide | ¹H NMR (400 MHz, DMSO-d₆) δ = 9.09 (s, 1H), 8.96 (dd, J = 2.0, 0.9 Hz, 1H), 8.66 (d, J = 2.3 Hz, 1H), 8.07 (d, J = 2.4 Hz, 1H), 7.77 (dd, J = 9.2, 1.9 Hz, 1H), 7.66 (s, 1H), 7.46 (dd, J = 9.1, 0.8 Hz, 1H), 7.39 (s, 1H), 6.10 (s, 2H), 5.36 (dd, J = 8.2, 4.6 Hz, 1H), 4.24 (d, J = 1.7 Hz, 1H), 4.03 (q, J = 8.0 Hz, 1H), 3.80 (s, 3H), 2.65 (ddd, J = 12.5, 8.2, 4.6 Hz, 1H), 2.45 (s, 3H), 2.32 (dt, J = 12.3, 3.7 Hz, 1H) | 420.1 |
| 77 | 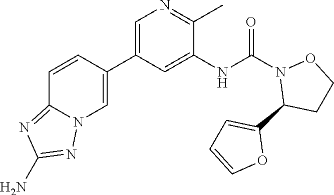 [SMILES: Cc1ncc(-c2ccc3nc(N)nn3c2)cc1NC(=O)N1OCC[C@H]1c1ccco1] | (S)-N-(5-(2-amino-[1,2,4]triazolo[1,5- a]pyridin-6-yl)-2-methylpyridin-3-yl)-3- (furan-2-yl)isoxazolidine-2-carboxamide | ¹H NMR (400 MHz, DMSO-d₆) δ = 9.18 (s, 1H), 8.97 (s, 1H), 8.67 (d, J = 2.2 Hz, 1H), 8.06 (d, J = 2.2 Hz, 1H), 7.77 (dt, J = 9.1, 1.5 Hz, 1H), 7.64 (s, 1H), 7.45 (d, J = 9.1 Hz, 1H), 6.46-6.36 (m, 2H), 6.10 (s, 2H), 5.48 (dd, J = 8.8, 5.0 Hz, 1H), 4.33 (td, J = 8.4, 3.5 Hz, 1H), 3.99 (q, J = 8.2 Hz, 1H), 3.30 (s, 1H), 2.73-2.66 (m, 1H), 2.45 (s, 3H) | 406.1 |
| 78 | 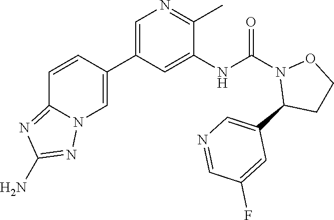 [SMILES: Cc1ncc(-c2ccc3nc(N)nn3c2)cc1NC(=O)N1OCC[C@H]1c1cncc(F)c1] | (S)-N-(5-(2-amino-[1,2,4]triazolo[1,5- a]pyridin-6-yl)-2-methylpyridin-3-yl)-3-(5- fluoropyridin-3-yl)isoxazolidine-2- carboxamide | ¹H NMR (400 MHz, DMSO-d₆) δ = 9.29 (s, 1H), 8.97 (d, J = 1.9 Hz, 1H), 8.68 (d, J = 2.3 Hz, 1H), 8.52 (t, J = 2.8 Hz, 2H), 8.09 (d, J = 2.2 Hz, 1H), 7.78 (dd, J = 9.2, 1.9 Hz, 1H), 7.71 (dt, J = 9.9, 2.3 Hz, 1H), 7.46 (d, J = 9.1 Hz, 1H), 6.11 (s, 2H), 5.54 (dd, J = 8.7, 5.6 Hz, 1H), 4.33 (td, J = 7.9, 3.2 Hz, 1H), 4.04 (q, J = 8.1 Hz, 1H), 2.93 (dtd, J = 11.9, 7.9, 3.2 Hz, 1H), 2.47 (s, 3H), 2.39-2.29 (m, 1H) | 435.2 |
| 79 | 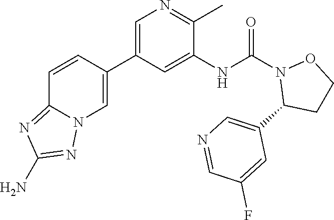 [SMILES: Cc1ncc(-c2ccc3nc(N)nn3c2)cc1NC(=O)N1OCC[C@@H]1c1cncc(F)c1] | (R)-N-(5-(2-amino-[1,2,4]triazolo[1,5- a]pyridin-6-yl)-2-methylpyridin-3-yl)-3-(5- fluoropyridin-3-yl)isoxazolidine-2- carboxamide | ¹H NMR (400 MHz, DMSO-d₆) δ = 9.30 (s, 1H), 8.98 (d, J = 1.9 Hz, 1H), 8.69 (d, J = 2.2 Hz, 1H), 8.52 (d, J = 3.1 Hz, 2H), 8.10 (d, J = 2.2 Hz, 1H), 7.79 (dd, J = 9.2, 1.9 Hz, 1H), 7.71 (dt, J = 9.9, 2.4 Hz, 1H), 7.46 (d, J = 9.1 Hz, 1H), 6.13 (s, 2H), 5.54 (dd, J = 8.7, 5.7 Hz, 1H), 4.33 (td, J = 8.0, 3.2 Hz, 1H), 4.04 (q, J = 8.1 Hz, 1H), 2.93 (dtd, J = 11.6, 7.9, 3.1 Hz, 1H), 2.48 (s, 3H), 2.38-2.31 (m, 1H) | 435.2 |
| 80 | 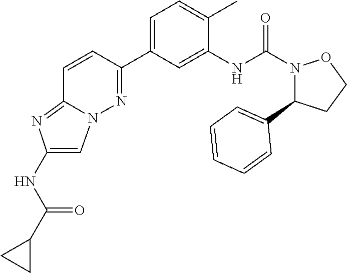 [SMILES: Cc1ccc(-c2ccc3nc(NC(=O)C4CC4)cn3n2)cc1NC(=O)N1OCC[C@H]1c1ccccc1] | (S)-N-(5-(2- (cyclopropanecarboxamido)imidazo[1,2- b]pyridazin-6-yl)-2-methylphenyl)-3- phenylisoxazolidine-2-carboxamide | ¹H NMR (400 MHz, DMSO-d₆) δ = 11.18 (s, 1H), 9.03 (s, 1H), 8.25 (s, 1H), 8.10-8.00 (m, 2H), 7.79 (dd, J = 8.0, 2.0 Hz, 1H), 7.72 (d, J = 9.5 Hz, 1H), 7.43-7.34 (m, 5H), 7.30- 7.25 (m, 1H), 5.42 (dd, J = 8.6, 5.6 Hz, 1H), 4.28 (td, J = 8.2, 7.8, 3.6 Hz, 1H), 4.00 (dt, J = 9.4, 7.3 Hz, 1H), 2.89 (dddd, J = 12.0, 8.6, 7.1, 3.1 Hz, 1H), 2.30 (s, 3H), 2.26 (tdd, J = 7.9, 3.9, 1.8 Hz, 1H), 2.01-1.93 (m, 1H), 0.88- 0.80 (m, 4H) | 483.3 |
| 81 | 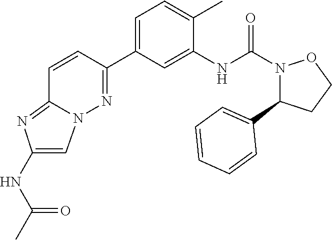 [SMILES: CC(=O)Nc1cn2nc(-c3ccc(C)c(NC(=O)N4OCC[C@H]4c4ccccc4)c3)ccc2n1] | (S)-N-(5-(2-acetamidoimidazo[1,2- b]pyridazin-6-yl)-2-methylphenyl)-3- phenylisoxazolidine-2-carboxamide | ¹H NMR (400 MHz, DMSO-d₆) δ = 10.90 (s, 1H), 9.03 (s, 1H), 8.27 (s, 1H), 8.09 (d, J = 2.0 Hz, 1H), 8.03 (dd, J= 9.4, 0.8 Hz, 1H), 7.79 (dd, J = 7.9, 2.1 Hz, 1H), 7.72 (d, J = 9.5 Hz, 1H), 7.43-7.34 (m, 5H), 7.31-7.25 (m, 1H), 5.43 (dd, J = 8.6, 5.7 Hz, 1H), 4.28 (td, J = 7.4, 2.8 Hz, 1H), 4.05-3.96 (m, 1H), 2.94-2.85 (m, 1H), 2.31 (s, 3H), 2.29- 2.23 (m, 1H), 2.11 (s, 3H) | 457.3 |
| 82 | 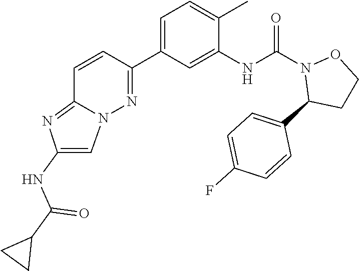 [SMILES: Cc1ccc(-c2ccc3nc(NC(=O)C4CC4)cn3n2)cc1NC(=O)N1OCC[C@H]1c1ccc(F)cc1] | (S)-N-(5-(2- (cyclopropanecarboxamido)imidazo[1,2- b]pyridazin-6-yl)-2-methylphenyl)-3-(4- fluorophenyl)isoxazolidine-2-carboxamide | ¹H NMR (400 MHz, DMSO-d₆) δ = 11.18 (s, 1H), 9.05 (s, 1H), 8.25 (s, 1H), 8.10-8.00 (m, 2H), 7.79 (dd, J = 7.9, 2.1 Hz, 1H), 7.72 (d, J = 9.5 Hz, 1H), 7.48-7.35 (m, 3H), 7.25- 7.13 (m, 2H), 5.43 (dd, J = 8.6, 5.8 Hz, 1H), 4.28 (td, J = 7.8, 3.4 Hz, 1H), 4.00 (dt, J = 9.4, 7.3 Hz, 1H), 2.88 (dddd, J = 12.1, 8.6, 7.2, 3.3 Hz, 1H), 2.30 (s, 3H), 2.28-2.21 (m, 1H), 1.96 (tt, J = 7.5, 4.9 Hz, 1H), 0.88-0.80 (m, 4H) | 501.3 |
| 83 | 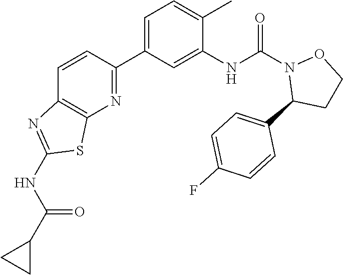 [SMILES: Cc1ccc(-c2ccc3nc(NC(=O)C4CC4)sc3n2)cc1NC(=O)N1OCC[C@H]1c1ccc(F)cc1] | (S)-N-(5-(2- (cyclopropanecarboxamido)thiazolo[5,4- b]pyridin-5-yl)-2-methylphenyl)-3-(4- fluorophenyl)isoxazolidine-2-carboxamide | ¹H NMR (400 MHz, DMSO-d₆) δ = 12.80 (s, 1H), 9.00 (s, 1H), 8.18-8.10 (m, 2H), 8.00 (d, J = 8.6 Hz, 1H), 7.86 (dd, J = 8.0, 2.0 Hz, 1H), 7.49-7.39 (m, 2H), 7.35 (d, J = 8.4 Hz, 1H), 7.23-7.14 (m, 2H), 5.43 (dd, J = 8.5, 5.6 Hz, 1H), 4.28 (td, J = 7.9, 3.3 Hz, 1H), 4.01 (dt, J = 9.4, 7.4 Hz, 1H), 2.88 (dddd, J = 12.3, 8.8, 7.3, 3.3 Hz, 1H), 2.30-2.19 (m, 4H), 2.03 (tt, J = 7.0, 5.3 Hz, 1H), 1.05-0.93 (m, 4H) | 518.2 |
| 84 | 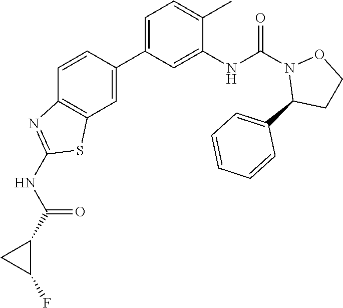 [SMILES: Cc1ccc(-c2ccc3nc(NC(=O)[C@H]4C[C@H]4F)sc3c2)cc1NC(=O)N1OCC[C@H]1c1ccccc1] | (S)-N-(5-(2-((1R,2R)-2-fluorocyclopropane- 1-carboxamido)benzo[d]thiazol-6-yl)-2- methylphenyl)-3-phenylisoxazolidine-2- carboxamide | ¹H NMR (400 MHz, DMSO-d₆) δ = 12.73 (s, 1H), 8.94 (s, 1H), 8.25 (d, J = 2.0 Hz, 1H), 7.82-7.76 (m, 2H), 7.70 (dd, J = 8.5, 1.9 Hz, 1H), 7.48 (dd, J = 7.9, 2.0 Hz, 1H), 7.43- 7.24 (m, 6H), 5.42 (dd, J = 8.6, 5.8 Hz, 1H), 5.15-4.94 (m, 1H), 4.28 (td, J = 8.1, 3.4 Hz, 1H), 4.00 (dt, J = 9.4, 7.6 Hz, 1H), 2.93-2.83 (m, 1H), 2.31-2.19 (m, 5H), 1.75 (dtd, J = 23.1, 6.8, 3.8 Hz, 1H), 1.31 (ddd, J = 13.0, 6.4, 2.8 Hz, 1H) | 517.3 |
| 85 | 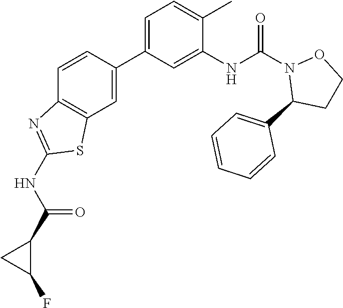 [SMILES: Cc1ccc(-c2ccc3nc(NC(=O)[C@@H]4C[C@@H]4F)sc3c2)cc1NC(=O)N1OCC[C@H]1c1ccccc1] | (S)-N-(5-(2-((1S,2S)-2-fluorocyclopropane- 1-carboxamido)benzo[d]thiazol-6-yl)-2- methylphenyl)-3-phenylisoxazolidine-2- carboxamide | ¹H NMR (400 MHz, DMSO-d₆) δ = 12.73 (s, 1H), 8.94 (s, 1H), 8.25 (d, J = 2.0 Hz, 1H), 7.82-7.76 (m, 2H), 7.70 (dd, J = 8.4, 1.9 Hz, 1H), 7.47 (dd, J = 7.9, 2.1 Hz, 1H), 7.43- 7.24 (m, 6H), 5.42 (dd, J = 8.6, 5.6 Hz, 1H), 5.04 (dtd, J = 65.7, 6.1, 3.8 Hz, 1H), 4.28 (td, J = 7.9, 3.3 Hz, 1H), 4.00 (dt, J = 9.4, 7.3 Hz, 1H), 2.88 (dtt, J = 10.3, 7.1, 3.2 Hz, 1H), 2.31-2.18 (m, 5H), 1.75 (dtd, J = 23.5, 6.9, 3.8 Hz, 1H), 1.32 (ddd, J = 12.9, 8.9, 6.1 Hz, 1H) | 517.3 |
| 86 | 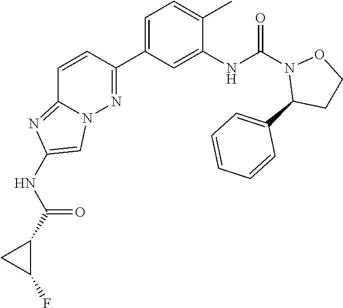 [SMILES: Cc1ccc(-c2ccc3nc(NC(=O)[C@H]4C[C@H]4F)cn3n2)cc1NC(=O)N1OCC[C@H]1c1ccccc1] | (S)-N-(5-(2-((1R,2R)-2-fluorocyclopropane- 1-carboxamido)imidazo[1,2-b]pyridazin-6- yl)-2-methylphenyl)-3-phenylisoxazolidine- 2-carboxamide | ¹H NMR (400 MHz, DMSO-d₆) δ = 11.24 (s, 1H), 9.04 (s, 1H), 8.27 (s, 1H), 8.10-8.01 (m, 2H), 7.79 (dd, J = 8.0, 2.0 Hz, 1H), 7.73 (d, J = 9.5 Hz, 1H), 7.44-7.33 (m, 5H), 7.30- 7.24 (m, 1H), 5.42 (dd, J = 8.8, 5.5 Hz, 1H), 5.04-4.85 (m, 1H), 4.28 (td, J = 7.8, 3.1 Hz, 1H), 4.04-3.96 (m, 1H), 2.94-2.84 (m, 1H), 2.30 (s, 4H), 2.20-2.12 (m, 1H), 1.73- 1.63 (m, 1H), 1.22-1.15 (m, 1H) | 501.3 |
| 87 | 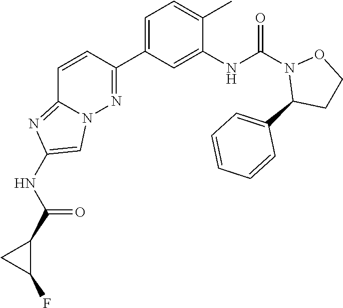 [SMILES: Cc1ccc(-c2ccc3nc(NC(=O)[C@@H]4C[C@@H]4F)cn3n2)cc1NC(=O)N1OCC[C@H]1c1ccccc1] | (S)-N-(5-(2-((1S,2S)-2-fluorocyclopropane- 1-carboxamido)imidazo[1,2-b]pyridazin-6- yl)-2-methylphenyl)-3-phenylisoxazolidine- 2-carboxamide | ¹H NMR (400 MHz, DMSO-d₆) δ = 11.23 (s, 1H), 9.04 (s, 1H), 8.27 (s, 1H), 8.10-8.01 (m, 2H), 7.79 (dd, J = 8.0, 2.0 Hz, 1H), 7.73 (d, J = 9.5 Hz, 1H), 7.44-7.33 (m, 5H), 7.30- 7.24 (m, 1H), 5.43 (dd, J = 8.6, 5.7 Hz, 1H), 5.05-4.84 (m, 1H), 4.28 (td, J = 8.0, 3.2 Hz, 1H), 4.01 (dt, J = 9.1, 7.4 Hz, 1H), 2.89 (dddd, J = 12.0, 8.7, 7.2, 3.2 Hz, 1H), 2.34- 2.22 (m, 4H), 2.16 (ddd, J = 9.2, 6.7, 4.7 Hz, 1H), 1.68 (dtd, J = 23.3, 6.7, 3.7 Hz, 1H), 1.19 (ddd, J = 12.3, 6.1, 2.9 Hz, 1H) | 501.3 |
| 88 | 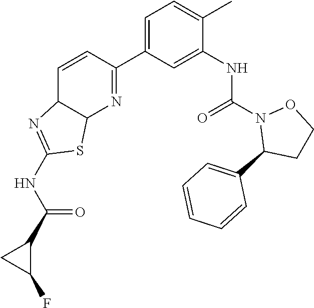 | (S)-N-(5-(2-((1S,2S)-2-fluorocyclopropane- 1-carboxamido)thiazolo[5,4-b]pyridin-5-yl)- 2-methylphenyl)-3-phenylisoxazolidine-2- carboxamide | ¹H NMR (400 MHz, DMSO-d₆) δ = 12.86 (s, 1H), 8.99 (s, 1H), 8.19-8.13 (m, 2H), 8.01 (d, J = 8.6 Hz, 1H), 7.86 (dd, J = 7.9, 2.0 Hz, 1H), 7.44-7.33 (m, 5H), 7.31-7.24 (m, 1H), 5.43 (dd, J = 8.6, 5.7 Hz, 1H), 5.17-4.96 (m, 1H), 4.28 (td, J = 7.9, 3.2 Hz, 1H), 4.01 (dt, J = 9.3, 7.4 Hz, 1H), 2.94-2.85 (m, 1H), 2.31-2.22 (m, 5H), 1.77 (dtd, J = 23.3, 6.8, 3.8 Hz, 1H), 1.33 (ddt, J = 12.6, 9.1, 6.4 Hz, 1H) | 518.3 |
| 89 | 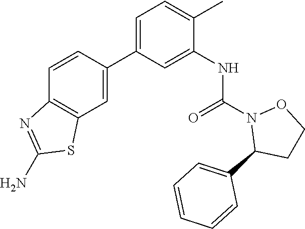 [SMILES: Cc1ccc(-c2ccc3nc(N)sc3c2)cc1NC(=O)N1OCC[C@H]1c1ccccc1] | (S)-N-(5-(2-aminobenzo[d]thiazol-6-yl)-2- methylphenyl)-3-phenylisoxazolidine-2- carboxamide | | 431.2 |
| 90 | 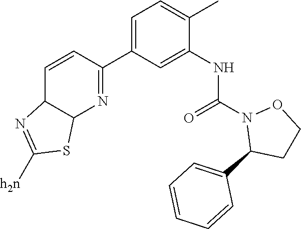 | (S)-N-(5-(2-aminothiazolo[5,4-b]pyridin-5- yl)-2-methylphenyl)-3-(4- fluorophenyl)isoxazolidine-2-carboxamide | | 450.1 |
| 91 | 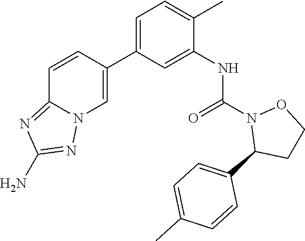 [SMILES: Cc1ccc([C@@H]2CCON2C(=O)Nc2cc(-c3ccc4nc(N)nn4c3)ccc2C)cc1] | (S)-N-(5-(2-amino-[1,2,4]triazolo[1,5- a]pyridin-6-yl)-2-methylphenyl)-3-(4- cyanophenyl)isoxazolidine-2-carboxamide | ¹H NMR (400 MHz, DMSO-d₆) δ = 9.05 (s, 1H), 8.81 (dd, J = 1.9, 0.9 Hz, 1H), 7.88-7.81 (m, 2H), 7.75-7.69 (m, 2H), 7.65-7.57 (m, 2H), 7.48 (dd, J = 7.9, 2.0 Hz, 1H), 7.42 (dd, J = 9.1, 0.9 Hz, 1H), 7.32 (d, J = 8.0 Hz, 1H), 6.05 (s, 2H), 5.51 (dd, J = 8.7, 5.8 Hz, 1H), 4.28 (td, J = 8.0, 3.0 Hz, 1H), 4.00 (q, J = 8.2 Hz, 1H), 2.99-2.89 (m, 1H), 2.26 (s, 3H), 2.24-2.18 (m, 1H) | 440.3 |
| 92 | 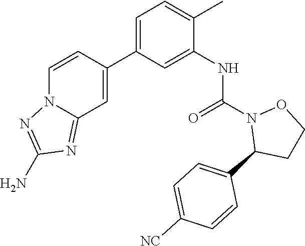 [SMILES: Cc1ccc(-c2ccn3nc(N)nc3c2)cc1NC(=O)N1OCC[C@H]1c1ccc(C#N)cc1] | (S)-N-(5-(2-amino-[1,2,4]triazolo[1,5- a]pyridin-7-yl)-2-methylphenyl)-3-(4- cyanophenyl)isoxazolidine-2-carboxamide | ¹H NMR (400 MHz, DMSO-d₆) δ = 9.06 (s, 1H), 8.56 (d, J = 7.0 Hz, 1H), 7.88-7.77 (m, 3H), 7.65-7.52 (m, 4H), 7.35 (d, J = 8.0 Hz, 1H), 7.15 (dd, J = 7.1, 2.0 Hz, 1H), 6.03 (s, 2H), 5.51 (dd, J = 8.7, 5.8 Hz, 1H), 4.28 (td, J = 8.0, 3.0 Hz, 1H), 4.01 (q, J = 8.2 Hz, 1H), 2.99-2.88 (m, 1H), 2.28 (s, 3H), 2.26-2.18 (m, 1H) | 440.2 |
| 93 | 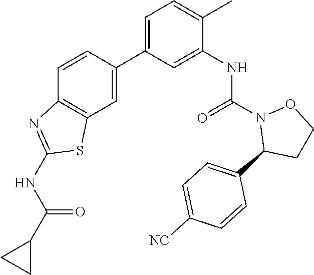 [SMILES: Cc1ccc(-c2ccc3nc(NC(=O)C4CC4)sc3c2)cc1NC(=O)N1OCC[C@H]1c1ccc(C#N)cc1] | (S)-3-(4-cyanophenyl)-N-(5-(2- (cyclopropanecarboxamido)benzo[d]thiazol- 6-yl)-2-methylphenyl)isoxazolidine-2- carboxamide | ¹H NMR (400 MHz, DMSO-d₆) δ = 12.67 (s, 1H), 9.03 (s, 1H), 8.23 (d, J = 1.8 Hz, 1H), 7.88-7.82 (m, 2H), 7.80- 7.73 (m, 2H), 7.69 (dd, J = 8.5, 1.9 Hz, 1H), 7.64-7.58 (m, 2H), 7.48 (dd, J = 7.9, 2.0 Hz, 1H), 7.32 (d, J = 8.0 Hz, 1H), 5.51 (dd, J = 8.7, 5.8 Hz, 1H), 4.28 (td, J = 7.9, 3.0 Hz, 1H), 4.05-3.96 (m, 1H), 2.99-2.89 (m, 1H), 2.27 (s, 3H), 2.25-2.18 (m, 1H), 2.01 (tt, J = 7.0, 4.2 Hz, 1H), 1.02- 0.90 (m, 4H) | 524.2 |
| 94 | 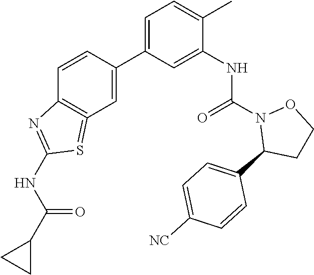 | (S)-3-(4-cyanophenyl)-N-(5-(2- (cyclopropanecarboxamido)thiazolo[5,4- b]pyridin-5-yl)-2- methylphenyl)isoxazolidine-2-carboxamide | ¹H NMR (400 MHz, DMSO-d₆) δ = 12.78 (s, 1H), 9.08 (s, 1H), 8.17-8.10 (m, 2H), 8.00 (d, J = 8.6 Hz, 1H), 7.89- 7.82 (m, 3H), 7.65-7.58 (m, 2H), 7.35 (d, J = 8.1 Hz, 1H), 5.52 (dd, J = 8.7, 5.8 Hz, 1H), 4.28 (td, J = 7.9, 3.0 Hz, 1H), 4.01 (q, J = 8.2 Hz, 1H), 3.00-2.90 (m, 1H), 2.28 (s, 3H), 2.26-2.19 (m, 1H), 2.03 (p, J = 6.4 Hz, 1H), 1.02- 0.95 (m, 4H) | 525.2 |
| 95 | 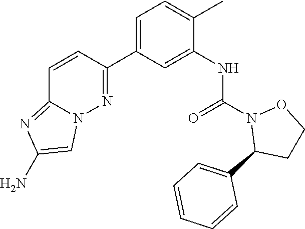 [SMILES: Cc1ccc(-c2ccc3nc(N)cn3n2)cc1NC(=O)N1OCC[C@H]1c1ccccc1] | (S)-N-(5-(2-aminoimidazo[ 1,2-b]pyridazin- 6-yl)-2-methylphenyl)-3- phenylisoxazolidine-2-carboxamide | ¹H NMR (400 MHz, DMSO-d₆) δ = 8.97 (s, 1H), 7.77 (d, J = 2.0 Hz, 1H), 7.59-7.54 (m, 1H), 7.45-7.32 (m, 6H), 7.31-7.20 (m, 3H), 5.44 (dq, J = 12.4, 6.7, 6.1 Hz, 1H), 4.27 (td, J = 7.8, 3.4 Hz, 1H), 4.10-3.98 (m, 1H), 2.97- 2.87 (m, 1H), 2.31-2.23 (m, 4H), 2.14 (s, 2H) | 415.3 |
| 96 | 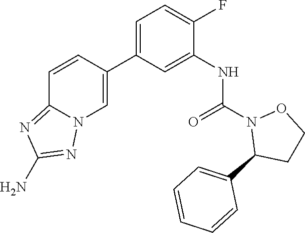 [SMILES: Nc1nc2ccc(-c3ccc(F)c(NC(=O)N4OCC[C@H]4c4ccccc4)c3)cn2n1] | (S)-N-(5-(2-amino-[1,2,4]triazolo[1,5- a]pyridin-6-yl)-2-fluorophenyl)-3- phenylisoxazolidine-2-carboxamide | ¹H NMR (400 MHz, DMSO-d₆) δ = 9.08 (s, 1H), 8.83 (dd, J = 2.0, 0.9 Hz, 1H), 7.95 (dd, J = 7.4, 2.4 Hz, 1H), 7.70 (dd, J = 9.1, 1.9 Hz, 1H), 7.55 (ddd, J = 8.6, 4.6, 2.5 Hz, 1H), 7.45-7.32 (m, 6H), 7.30-7.24 (m, 1H), 6.08 (s, 2H), 5.40 (dd, J = 8.6, 5.8 Hz, 1H), 4.28 (td, J = 7.9, 3.1 Hz, 1H), 3.96 (dt, J = 9.2, 7.3 Hz, 1H), 2.89 (dddd, J = 11.9, 9.0, 6.9, 3.0 Hz, 1H), 2.25 (dddd, J = 12.2, 9.6, 7.8, 5.9 Hz, 1H) | 419.2 |
| 97 | 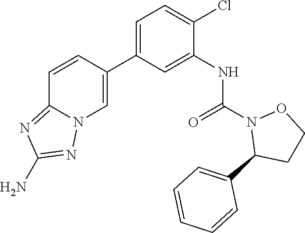 [SMILES: Nc1nc2ccc(-c3ccc(Cl)c(NC(=O)N4OCC[C@H]4c4ccccc4)c3)cn2n1] | (S)-N-(5-(2-amino-[1,2,4]triazolo[1,5- a]pyridin-6-yl)-2-chlorophenyl)-3- phenylisoxazolidine-2-carboxamide | ¹H NMR (400 MHz, DMSO-d₆) δ = 9.08 (s, 1H), 8.83 (dd, J = 2.0, 0.9 Hz, 1H), 7.95 (dd, J = 7.4, 2.4 Hz, 1H), 7.70 (dd, J = 9.1, 1.9 Hz, 1H), 7.55 (ddd, J = 8.6, 4.6, 2.5 Hz, 1H), 7.46-7.32 (m, 6H), 7.31-7.24 (m, 1H), 6.08 (s, 2H), 5.40 (dd, J = 8.6, 5.8 Hz, 1H), 4.28 (td, J = 7.9, 3.1 Hz, 1H), 3.96 (dt, J = 9.2, 7.3 Hz, 1H), 2.89 (dddd, J = 11.9, 9.0, 6.9, 3.0 Hz, 1H), 2.25 (dddd, J = 12.2, 9.6, 7.8, 5.9 Hz, 1H) | 435.2 |
| 98 | 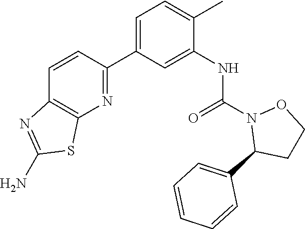 [SMILES: Cc1ccc(-c2ccc3nc(N)sc3n2)cc1NC(=O)N1OCC[C@H]1c1ccccc1] | (S)-N-(5-(2-aminothiazolo[5,4-b]pyridin-5- yl)-2-methylphenyl)-3-phenylisoxazolidine- 2-carboxamide | ¹H NMR (400 MHz, DMSO-d₆) δ = 8.94 (s, 1H), 8.08 (d, J = 1.9 Hz, 1H), 7.83 (s, 2H), 7.79-7.72 (m, 2 H), 7.65 (d, J = 8.4 Hz, 1H), 7.43-7.34 (m, 4H), 7.32-7.25 (m, 2H), 5.42 (dd, J = 8.6, 5.6 Hz, 1H), 4.27 (td, J = 7.9, 3.2 Hz, 1H), 4.04-3.95 (m, 1H), 2.94-2.83 (m, 1H), 2.27 (s, 4H) | 432.2 |
| 99 | 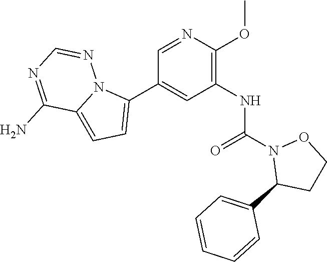 [SMILES: COc1ncc(-c2ccc3c(N)ncnn23)cc1NC(=O)N1OCC[C@H]1c1ccccc1] | (S)-N-(5-(4-aminopyrrolo[2,1- f][1,2,4]triazin-7-yl)-2-methoxypyridin-3- yl)-3-phenylisoxazolidine-2-carboxamide | ¹H NMR (400 MHz, DMSO-d₆) δ = 8.90 (d, J = 2.2 Hz, 1H), 8.53-8.46 (m, 2H), 7.90 (s, 1H), 7.78 (s, 2 H), 7.41-7.34 (m, 4H), 7.28 (ddt, J = 8.6, 5.9, 2.1 Hz, 1H), 7.02-6.93 (m, 2H), 5.40 (dd, J = 8.5, 6.1 Hz, 1H), 4.31 (td, J = 7.8, 3.1 Hz, 1H), 4.04-3.94 (m, 4H), 2.91 (dddd, J = 11.9, 8.5, 6.8, 3.1 Hz, 1H), 2.29-2.20 (m, 1H) | 432.2 |
| 100 | 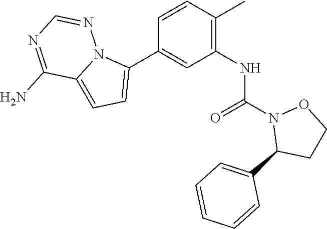 [SMILES: Cc1ccc(-c2ccc3c(N)ncnn23)cc1NC(=O)N1OCC[C@H]1c1ccccc1] | (S)-N-(5-(4-aminopyrrolo[2,1- f][1,2,4]triazin-7-yl)-2-methylphenyl)-3- phenylisoxazolidine-2-carboxamide | ¹H NMR (400 MHz, DMSO-d₆) δ = 8.98 (s, 1H), 8.06 (d, J = 1.8 Hz, 1H), 7.91 (s, 1H), 7.83 (dd, J = 8.0, 1.9 Hz, 1H), 7.73 (s, 2H), 7.43-7.33 (m, 4H), 7.31- 7.24 (m, 2H), 7.01-6.94 (m, 2H), 5.41 (dd, J = 8.7, 5.6 Hz, 1H), 4.27 (td, J = 7.9, 3.3 Hz, 1H), 3.98 (dt, J = 9.0, 7.4 Hz, 1H), 2.92-2.82 (m, 1H), 2.31- 2.19 (m, 4H) | 415.2 |
| 101 | 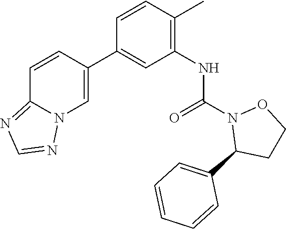 [SMILES: Cc1ccc(-c2ccc3ncnn3c2)cc1NC(=O)N1OCC[C@H]1c1ccccc1] | (S)-N-(5-([1,2,4]triazolo[1,5-a]pyridin-6- yl)-2-methylphenyl)-3-phenylisoxazolidine- 2-carboxamide | ¹H NMR (400 MHz, DMSO-d₆) δ = 9.22 (dd, J = 1.9, 1.0 Hz, 1H), 8.98 (s, 1H), 8.49 (s, 1H), 7.95 (dd, J = 9.4, 1.8 Hz, 1H), 7.89 (dd, J = 9.3, 1.0 Hz, 1H), 7.79 (d, J = 2.0 Hz, 1H), 7.53 (dd, J = 7.9, 2.1 Hz, 1H), 7.41-7.29 (m, 5H), 7.27-7.19 (m, 1H), 5.38 (dd, J = 8.7, 5.7 Hz, 1H), 4.24 (td, J = 8.0, 3.2 Hz, 1H), 3.96 (dt, J = 9.0, 7.4 Hz, 1H), 2.90- 2.79 (m, 1H), 2.25 (s, 3H), 2.23-2.18 (m, 1H) | 400.2 |
| 102 | 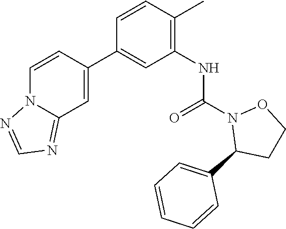 [SMILES: Cc1ccc(-c2ccn3ncnc3c2)cc1NC(=O)N1OCC[C@H]1c1ccccc1] | (S)-N-(5-([1,2,4]triazolo[1,5-a]pyridin-7- yl)-2-methylphenyl)-3-phenylisoxazolidine- 2-carboxamide | ¹H NMR (400 MHz, DMSO-d₆) δ = 9.04 (s, 1H), 9.01 (dd, J = 7.2, 0.8 Hz, 1H), 8.52 (s, 1H), 8.10 (dd, J = 2.0, 0.9 Hz, 1H), 7.91 (d, J = 2.0 Hz, 1H), 7.65 (dd, J = 7.9, 2.1 Hz, 1H), 7.52 (dd, J = 7.2, 2.0 Hz, 1H), 7.43-7.35 (m, 5H), 7.31-7.25 (m, 1H), 5.43 (dd, J = 8.6, 5.7 Hz, 1H), 4.29 (td, J = 7.9, 3.1 Hz, 1H), 4.01 (dt, J = 9.1, 7.4 Hz, 1H), 2.89 (dtd, J = 11.8, 8.0, 3.1 Hz, 2H), 2.30 (s, 3H), 2.27-2.21 (m, 1H) | 400.2 |
| 103 | 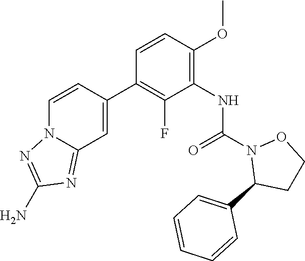 [SMILES: COc1ccc(-c2ccn3nc(N)nc3c2)c(F)c1NC(=O)N1OCC[C@H]1c1ccccc1] | (S)-N-(3-(2-amino-[1,2,4]triazolo[1,5- a]pyridin-7-yl)-2-fluoro-6-methoxyphenyl)- 3-phenylisoxazolidine-2-carboxamide | ¹H NMR (400 MHz, DMSO-d₆) δ = 8.80 (s, 1H), 8.60 (d, J = 7.0 Hz, 1H), 7.58 (t, J = 8.8 Hz, 1H), 7.45 (d, J = 1.9 Hz, 1H), 7.41-7.32 (m, 4H), 7.30-7.23 (m, 1H), 7.04 (td, J = 6.8, 3.3 Hz, 2H), 5.37 (dd, J = 8.7, 5.5 Hz, 1H), 4.25 (td, J = 7.9, 3.2 Hz, 1H), 3.96-3.83 (m, 4H), 2.86 (ddt, J = 12.5, 8.8, 4.1 Hz, 1H), 2.23 (dtd, J = 16.1, 8.1, 4.1 Hz, 1H) | 449.2 |
| 104 | 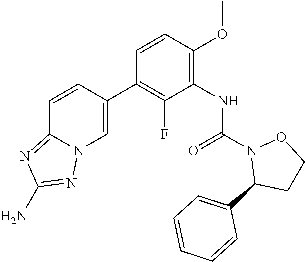 [SMILES: COc1ccc(-c2ccc3nc(N)nn3c2)c(F)c1NC(=O)N1OCC[C@H]1c1ccccc1] | (S)-N-(3-(2-amino-[1,2,4]triazolo[1,5- a]pyridin-6-yl)-2-fluoro-6-methoxyphenyl)- 3-phenylisoxazolidine-2-carboxamide | ¹H NMR (400 MHz, DMSO-d₆) δ = 8.79 (s, 1H), 8.66- 8.62 (m, 1H), 7.58-7.48 (m, 2H), 7.45-7.32 (m, 5H), 7.30-7.22 (m, 1H), 7.06-7.00 (m, 1H), 6.07 (s, 2H), 5.37 (dd, J = 8.6, 5.5 Hz, 1H), 4.25 (td, J = 7.9, 3.1 Hz, 1H), 3.85 (s, 4H), 2.90-2.79 (m, 1H), 2.28-2.17 (m, 1H) | 449.2 |
| 105 | 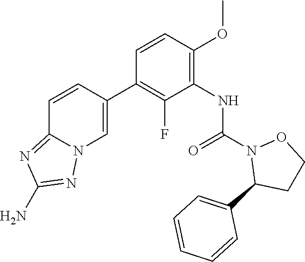 [SMILES: COc1ccc(-c2ccc3nc(N)nn3c2)c(F)c1NC(=O)N1OCC[C@H]1c1ccccc1] | (S)-N-(3-(2-amino-[1,2,4]triazolo[1,5- a]pyridin-6-yl)-6-chloro-2-fluorophenyl)-3- phenylisoxazolidine-2-carboxamide | ¹H NMR (400 MHz, DMSO-d₆) δ = 9.37 (s, 1H), 8.77- 8.73 (m, 1H), 7.63-7.54 (m, 2H), 7.52-7.43 (m, 2H), 7.41-7.34 (m, 4H), 7.30-7.25 (m, 1H), 6.13 (s, 2H), 5.38 (dd, J = 8.7, 5.6 Hz, 1H), 4.30 (td, J = 8.0, 3.1 Hz, 1H), 3.95 (q, J = 8.2 Hz, 1H), 2.88 (ddd, J = 15.5, 11.9, 3.2 Hz, 1H), 2.25 (dt, J = 9.3, 5.3 Hz, 1H) | 453.2 |
| 106 | 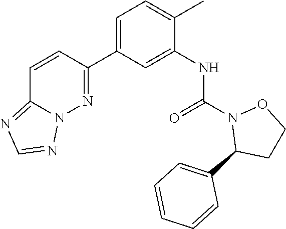 [SMILES: Cc1ccc(-c2ccc3ncnn3n2)cc1NC(=O)N1OCC[C@H]1c1ccccc1] | (S)-N-(5-([1,2,4]triazolo[4,3-b]pyridazin-6- yl)-2-methylphenyl)-3-phenylisoxazolidine- 2-carboxamide | | 401.2 |
| 107 | 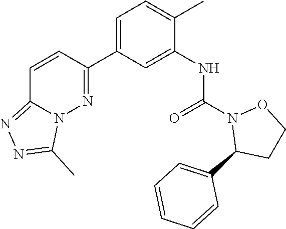 [SMILES: Cc1ccc(-c2ccc3nnc(C)n3n2)cc1NC(=O)N1OCC[C@H]1c1ccccc1] | (S)-N-(2-methyl-5-(3-methyl- [1,2,4]triazolo[4,3-b]pyridazin-6- yl)phenyl)-3-phenylisoxazolidine-2- carboxamide | ¹H NMR (400 MHz, DMSO-d₆) δ = 9.11 (s, 1H), 8.38 (d, J = 9.8 Hz, 1H), 8.15 (d, J = 1.9 Hz, 1H), 7.91-7.84 (m, 2H), 7.45 (d, J = 8.1 Hz, 1H), 7.44-7.35 (m, 4H), 7.32- 7.25 (m, 1H), 5.43 (dd, J = 8.6, 5.7 Hz, 1H), 4.30 (td, J = 8.0, 3.1 Hz, 1H), 4.01 (dt, J = 9.2, 7.4 Hz, 1H), 2.90 (dddd, J = 12.0, 8.6, 7.1, 3.2 Hz, 1H), 2.33 (s, 3H), 2.26 (dddd, J = 12.1, 9.4, 7.9, 5.6 Hz, 1H) | 415.3 |
| 108 | 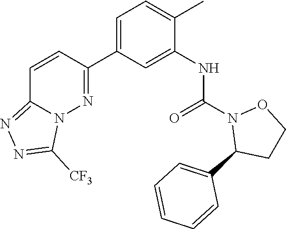 [SMILES: Cc1ccc(-c2ccc3nnc(C(F)(F)F)n3n2)cc1NC(=O)N1OCC[C@H]1c1ccccc1] | (S)-N-(2-methyl-5-(3-(trifluoromethyl)- [1,2,4]triazolo[4,3-b]pyridazin-6- yl)phenyl)-3-phenylisoxazolidine-2- carboxamide | ¹H NMR (400 MHz, DMSO-d₆) δ = 9.15 (s, 1H), 8.66 (d, J = 9.9 Hz, 1H), 8.20-8.15 (m, 2H), 7.88 (dd, J = 8.0, 2.0 Hz, 1H), 7.49 (d, J = 8.1 Hz, 1H), 7.45-7.35 (m, 4H), 7.32- 7.25 (m, 1H), 5.43 (dd, J = 8.6, 5.6 Hz, 1H), 4.30 (td, J = 7.9, 3.2 Hz, 1H), 4.00 (dt, J = 9.1, 7.4 Hz, 1H), 2.90 (dddd, J = 12.1, 8.7, 7.2, 3.2 Hz, 1H), 2.34 (s, 3H), 2.29-2.23 (m, 1H) | 469.2 |
| 109 | 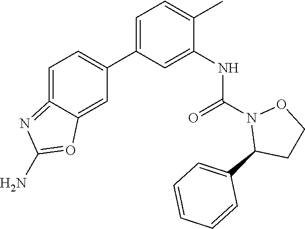 | (S)-N-(5-(2-aminobenzo[d]oxazol-6-yl)-2- methylphenyl)-3-phenylisoxazolidine-2- carboxamide | ¹H NMR (400 MHz, DMSO-d₆) δ = 8.92 (s, 1H), 7.79 (s, 2H), 7.69 (d, J = 1.9 Hz, 1H), 7.62 (d, J = 1.6 Hz, 1H), 7.43- 7.33 (m, 6H), 7.27 (dd, J = 7.9, 5.3 Hz, 3H), 5.42 (dd, J = 8.6, 5.6 Hz, 1H), 4.27 (td, J = 7.9, 3.2 Hz, 1H), 3.99 (dt, J = 9.2, 7.5 Hz, 1H), 2.87 (dtt, J = 10.3, 7.2, 3.2 Hz, 1H), 2.30- 2.19 (m, 4H) | 415.3 |
| 110 | 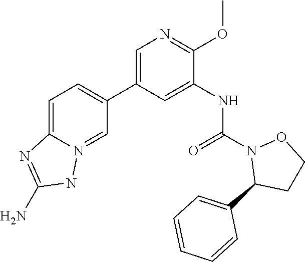 | (S)-N-(5-(2-aminobenzo[d]oxazol-6-yl)-2- methoxypyridin-3-yl)-3- phenylisoxazolidine-2-carboxamide | ¹H NMR (400 MHz, DMSO-d₆) δ = 8.52 (d, J = 2.3 Hz, 1H), 8.46 (s, 1H), 8.17 (d, J = 2.3 Hz, 1H), 7.70 (s, 2H), 7.61 (d, J = 1.7 Hz, 1H), 7.41-7.32 (m, 5H), 7.31-7.24 (m, 2H), 5.39 (dd, J = 8.5, 6.2 Hz, 1H), 4.32 (td, J = 7.8, 3.0 Hz, 1H), 4.01 (s, 4H), 2.90 (dtt, J = 9.4, 6.9, 3.4 Hz, 1H), 2.32-2.21 (m, 1H) | 432.2 |
| 111 | 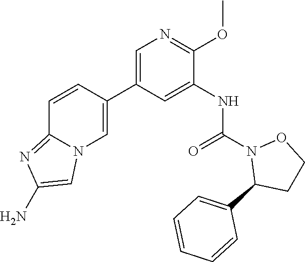 [SMILES: COc1ncc(-c2ccc3nc(N)cn3c2)cc1NC(=O)N1OCC[C@H]1c1ccccc1] | (S)-N-(5-(2-aminoimidazo[ 1,2-a]pyridin-6- yl)-2-methoxypyridin-3-yl)-3- phenylisoxazolidine-2-carboxamide | ¹H NMR (400 MHz, DMSO-d₆) δ = 8.80 (s, 1H), 8.57- 8.43 (m, 2H), 8.41-8.28 (m, 1H), 8.20 (dd, J = 14.5, 2.4 Hz, 1H), 7.43-7.32 (m, 4H), 7.29 (td, J = 6.0, 2.7 Hz, 1H), 7.22 (s, 1H), 7.10 (s, 1H), 6.97 (s, 1H), 5.36 (dd, J = 8.5, 6.4 Hz, 1H), 4.93 (s, 1H), 4.33 (td, J = 7.8, 2.9 Hz, 1H), 4.07-3.90 (m, 4H), 2.94-2.86 (m, 1H), 2.28 (ddt, J = 17.8, 12.8, 7.2 Hz, 1H) | 431.2 |
| 112 | 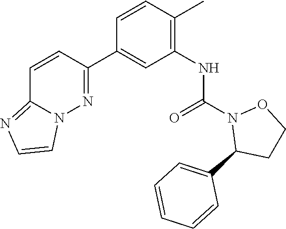 [SMILES: Cc1ccc(-c2ccc3nccn3n2)cc1NC(=O)N1OCC[C@H]1c1ccccc1] | (S)-N-(5-(imidazo[1,2-b]pyridazin-6-yl)-2- methylphenyl)-3-phenylisoxazolidine-2- carboxamide | ¹H NMR (400 MHz, DMSO-d₆) δ = 9.02 (s, 1H), 8.34 (t, J = 1.0 Hz, 1H), 8.19 (dd, J = 9.6, 0.8 Hz, 1H), 8.14 (d, J = 1.9 Hz, 1H), 7.83-7.73 (m, 3H), 7.44-7.34 (m, 5H), 7.31- 7.25 (m, 1H), 5.42 (dd, J = 8.6, 5.8 Hz, 1H), 4.29 (td, J = 8.0, 3.2 Hz, 1H), 4.00 (dt, J = 9.1, 7.4 Hz, 1H), 2.95-2.84 (m, 1H), 2.35-2.22 (m, 4H) | 400.3 |
| 113 | 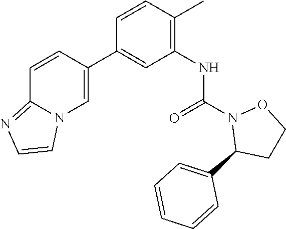 [SMILES: Cc1ccc(-c2ccc3nccn3c2)cc1NC(=O)N1OCC[C@H]1c1ccccc1] | (S)-N-(5-(imidazo[1,2-a]pyridin-6-yl)-2- methylphenyl)-3-phenylisoxazolidine-2- carboxamide | ¹H NMR (400 MHz, DMSO-d₆) δ = 8.96 (s, 1H), 8.88 (dd, J = 1.9, 1.0 Hz, 1H), 7.97 (t, J = 1.0 Hz, 1H), 7.76 (d, J = 2.0 Hz, 1H), 7.66-7.61 (m, 1H), 7.59 (d, J = 1.2 Hz, 1H), 7.53 (dd, J = 9.4, 1.9 Hz, 1H), 7.47-7.32 (m, 6H), 7.30- 7.24 (m, 1H), 5.42 (dd, J = 8.6, 5.7 Hz, 1H), 4.29 (td, J = 8.0, 3.2 Hz, 1H), 3.99 (dt, J = 9.2, 7.4 Hz, 1H), 2.88 (dddd, J = 12.0, 8.7, 7.1, 3.1 Hz, 1H), 2.31-2.21 (m, 4H) | 399.3 |
| 114 | 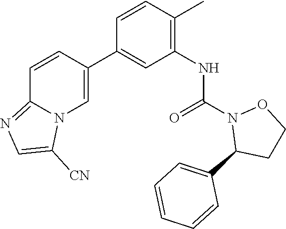 [SMILES: Cc1ccc(-c2ccc3ncc(C#N)n3c2)cc1NC(=O)N1OCC[C@H]1c1ccccc1] | (S)-N-(5-(3-cyanoimidazo[1,2-a]pyridin-6- yl)-2-methylphenyl)-3-phenylisoxazolidine- 2-carboxamide | ¹H NMR (400 MHz, DMSO-d₆) δ = 9.01 (s, 1H), 8.74 (t, J = 1.3 Hz, 1H), 8.47 (s, 1H), 7.95-7.87 (m, 2H), 7.84 (d, J = 2.0 Hz, 1H), 7.57 (dd, J = 7.9, 2.1 Hz, 1H), 7.43-7.32 (m, 5H), 7.30-7.24 (m, 1H), 5.42 (dd, J = 8.6, 5.7 Hz, 1H), 4.28 (td, J = 7.9, 3.1 Hz, 1H), 4.00 (dt, J = 9.1, 7.4 Hz, 1H), 2.89 (ddt, J = 12.4, 8.8, 4.3 Hz, 1H), 2.29 (s, 4H) | 424.2 |
| 115 | 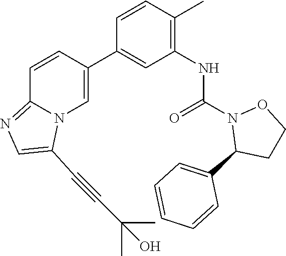 [SMILES: Cc1ccc(-c2ccc3ncc(C#CC(C)(C)O)n3c2)cc1NC(=O)N1OCC[C@H]1c1ccccc1] | (S)-N-(5-(3-(3-hydroxy-3-methylbut-1-yn- 1-yl)imidazo[1,2-a]pyridin-6-yl)-2- methylphenyl)-3-phenylisoxazolidine-2- carboxamide | ¹H NMR (400 MHz, DMSO-d₆) δ = 8.98 (s, 1H), 8.50 (d, J = 1.5 Hz, 1H), 7.92 (s, 1H), 7.82-7.76 (m, 2H), 7.72 (dd, J = 9.3, 1.8 Hz, 1H), 7.49 (dd, J = 7.9, 2.0 Hz, 1H), 7.43- 7.34 (m, 5H), 7.31-7.25 (m, 1H), 5.71 (s, 1H), 5.41 (dd, J = 8.6, 5.7 Hz, 1H), 4.29 (td, J = 7.9, 3.1 Hz, 1H), 3.99 (q, J = 8.1 Hz, 1H), 2.90-2.85 (m, 1H), 2.30 (s, 4H), 1.55 (d, J = 1.1 Hz, 6H) | 481.3 |
| 116 | 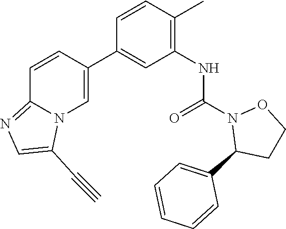 [SMILES: C#Cc1cnc2ccc(-c3ccc(C)c(NC(=O)N4OCC[C@H]4c4ccccc4)c3)cn12] | (S)-N-(5-(3-ethynylimidazo[1,2-a]pyridin- 6-yl)-2-methylphenyl)-3- phenylisoxazolidine-2-carboxamide | ¹H NMR (400 MHz, DMSO-d₆) δ = 8.99 (s, 1H), 8.50 (dd, J = 1.9, 1.0 Hz, 1H), 7.98 (s, 1H), 7.81-7.76 (m, 2H), 7.70 (dd, J = 9.4, 1.9 Hz, 1H), 7.50 (dd, J = 7.9, 2.0 Hz, 1H), 7.43-7.34 (m, 5H), 7.30-7.24 (m, 1H), 5.42 (dd, J = 8.6, 5.6 Hz, 1H), 5.12 (s, 1H), 4.28 (td, J = 8.0, 3.2 Hz, 1H), 4.00 (q, J = 8.1 Hz, 1H), 2.94-2.83 (m, 1H), 2.29 (s, 4H) | 423.2 |
| 117 | 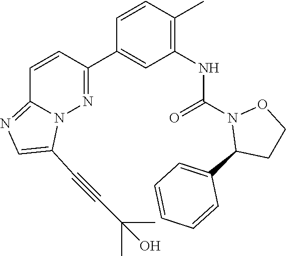 [SMILES: Cc1ccc(-c2ccc3ncc(C#CC(C)(C)O)n3n2)cc1NC(=O)N1OCC[C@H]1c1ccccc1] | (S)-N-(5-(3-(3-hydroxy-3-methylbut-1-yn- 1-yl)imidazo[1,2-b]pyridazin-6-yl)-2- methylphenyl)-3-phenylisoxazolidine-2- carboxamide | ¹H NMR (400 MHz, DMSO-d₆) δ = 9.04 (s, 1H), 8.28- 8.21 (m, 2H), 8.02 (s, 1H), 7.93-7.85 (m, 2H), 7.46-7.33 (m, 5H), 7.31-7.25 (m, 1H), 5.74-5.53 (m, 1H), 5.42 (dd, J = 8.6, 5.7 Hz, 1H), 4.29 (td, J = 7.9, 3.1 Hz, 1H), 3.98 (dt, J = 9.3, 7.4 Hz, 1H), 2.93-2.81 (m, 1H), 2.36-2.21 (m, 4H), 1.54 (d, J = 1.3 Hz, 6H) | 482.2 |
| 118 | 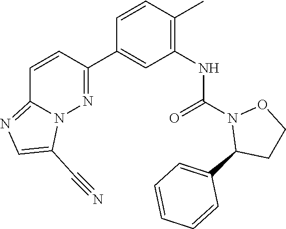 [SMILES: Cc1ccc(-c2ccc3ncc(C#N)n3n2)cc1NC(=O)N1OCC[C@H]1c1ccccc1] | (S)-N-(5-(3-cyanoimidazo[1,2-b]pyridazin- 6-yl)-2-methylphenyl)-3- phenylisoxazolidine-2-carboxamide | ¹H NMR (400 MHz, DMSO-d₆) δ = 9.11 (s, 1H), 8.60 (s, 1H), 8.44 (d, J = 9.6 Hz, 1H), 8.15 (d, J = 2.0 Hz, 1H), 8.09 (d, J = 9.6 Hz, 1H), 7.87 (dd, J = 7.9, 2.0 Hz, 1H), 7.47 (d, J = 8.0 Hz, 1H), 7.43-7.34 (m, 4H), 7.30-7.25 (m, 1H), 5.43 (dd, J = 8.6, 5.7 Hz, 1H), 4.29 (td, J = 8.0, 3.2 Hz, 1H), 4.01 (dt, J = 9.1, 7.4 Hz, 1H), 2.90 (dddd, J = 12.0, 8.5, 7.1, 3.2 Hz, 1H), 2.37-2.20 (m, 4H) | 425.2 |
| 119 | 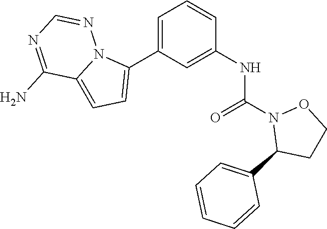 [SMILES: Nc1ncnn2c(-c3cccc(NC(=O)N4OCC[C@H]4c4ccccc4)c3)ccc12] | (S)-N-(3-(4-aminopyrrolo[2,1- f][1,2,4]triazin-7-yl)phenyl)-3- phenylisoxazolidine-2-carboxamide | ¹H NMR (400 MHz, DMSO-d₆) δ = 9.45 (s, 1H), 8.24 (t, J = 1.9 Hz, 1H), 7.92 (s, 1H), 7.76 (dt, J = 7.8, 1.2 Hz, 3H), 7.61 (ddd, J = 8.2, 2.2, 1.1 Hz, 1H), 7.43-7.32 (m, 5H), 7.31-7.24 (m, 1H), 7.00 (d, J = 4.5 Hz, 1H), 6.93 (d, J = 4.6 Hz, 1H), 5.43 (dd, J = 8.6, 5.8 Hz, 1H), 4.26 (td, J = 7.9, 3.1 Hz, 1H), 3.97-3.84 (m, 1H), 2.86 (dtt, J = 10.1, 7.0, 3.5 Hz, 1H), 2.23 (dddd, J = 12.1, 9.5, 7.9, 5.8 Hz, 1H) | 401.3 |
| 120 | 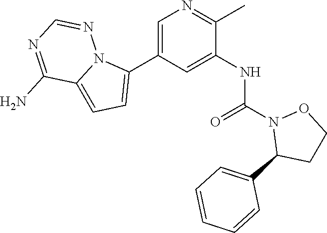 [SMILES: Cc1ncc(-c2ccc3c(N)ncnn23)cc1NC(=O)N1OCC[C@H]1c1ccccc1] | (S)-N-(5-(4-aminopyrrolo[2,1- f][1,2,4]triazin-7-yl)-2-methylpyridin-3-yl)- 3-phenylisoxazolidine-2-carboxamide | 1H NMR (400 MHz, DMSO-d6) 8 = 9.21 (s, 1H), 8.94 (d, J = 2.1 Hz, 1H), 8.43 (d, J = 2.1 Hz, 1H), 7.95 (s, 1H), 7.44- 7.34 (m, 4H), 7.32-7.24 (m, 1H), 7.12 (d, J = 4.6 Hz, 1H), 7.03 (d, J = 4.6 Hz, 1H), 5.41 (dd, J = 8.6, 5.7 Hz, 1H), 4.30 (td, J = 8.0, 3.0 Hz, 1H), 3.99 (dt, J = 9.2, 7.4 Hz, 1H), 2.90 (dddd, J = 11.9, 9.6, 7.0, 3.0 Hz, 1H), 2.46 (s, 3H), 2.25 (dddd, J = 12.2, 9.5, 8.0, 5.8 Hz, 1H) | 416.3 |
| 121 | 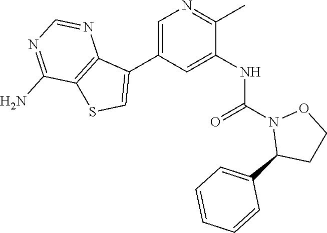 [SMILES: Cc1ncc(-c2csc3c(N)ncnc23)cc1NC(=O)N1OCC[C@H]1c1ccccc1] | (S)-N-(5-(4-aminothieno[3,2-d]pyrimidine- 7-y1)-2-methylpyridin-3-yl)-3- phenylisoxazolidine-2-carboxamide | ¹H NMR (400 MHz, DMSO-d₆) δ = 9.30 (s, 1H), 9.01 (d, J = 2.1 Hz, 1H), 8.54 (d, J = 3.7 Hz, 2H), 8.49 (d, J = 2.0 Hz, 1H), 7.67 (s, 2H), 7.50-7.40 (m, 4H), 7.37-7.29 (m, 1H), 5.47 (dd, J = 8.6, 5.7 Hz, 1H), 4.37 (td, J = 7.9, 3.0 Hz, 1H), 4.05 (dt, J = 9.5, 7.5 Hz, 1H), 2.96 (dddd, J = 12.0, 8.7, 7.1, 3.1 Hz, 1H), 2.54 (s, 3H), 2.32 (dddd, J = 12.1, 9.5, 7.8, 5.7 Hz, 1H) | 433.2 |
| 122 | 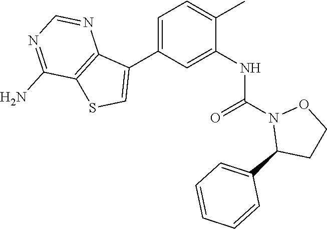 [SMILES: Cc1ccc(-c2csc3c(N)ncnc23)cc1NC(=O)N1OCC[C@H]1c1ccccc1] | (S)-N-(5-(4-aminothieno[3,2-d]pyrimidine- 7-yl)-2-methylphenyl)-3- phenylisoxazolidine-2-carboxamide | ¹H NMR (400 MHz, DMSO-d₆) δ = 9.03 (s, 1H), 8.46 (s, 1H), 8.30 (s, 1H), 8.05 (d, J = 1.8 Hz, 1H), 7.85 (dd, J = 7.8, 1.9 Hz, 1H), 7.51 (s, 2H), 7.45-7.34 (m, 4H), 7.33- 7.25 (m, 2H), 5.41 (dd, J = 8.6, 5.6 Hz, 1H), 4.28 (td, J = 8.0, 3.2 Hz, 1H), 3.98 (dt, J = 9.2, 7.5 Hz, 1H), 2.88 (tq, J = 10.4, 3.2 Hz, 1H), 2.27 (s, 3H), 2.26-2.20 (m, 1H) | 432.2 |
| 123 | 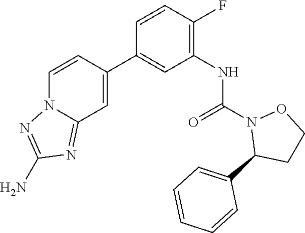 [SMILES: Nc1nc2cc(-c3ccc(F)c(NC(=O)N4OCC[C@H]4c4ccccc4)c3)ccn2n1] | (S)-N-(5-(2-amino-[1,2,4]triazolo[1,5- a]pyridin-7-yl)-2-fluorophenyl)-3- phenylisoxazolidine-2-carboxamide | ¹H NMR (400 MHz, DMSO-d₆) δ = 9.10 (s, 1H), 8.59 (d, J = 7.0 Hz, 1H), 8.05 (dd, J = 7.4, 2.4 Hz, 1H), 7.64 (ddd, J = 8.7, 4.6, 2.5 Hz, 1H), 7.58 (d, J = 1.9 Hz, 1H), 7.43-7.33 (m, 5H), 7.31-7.24 (m, 1H), 7.14 (dd, J = 7.0, 2.0 Hz, 1H), 6.06 (s, 2H), 5.40 (dd, J = 8.6, 5.9 Hz, 1H), 4.29 (td, J= 7.9, 3.1 Hz, 1H), 3.97 (dt, J = 9.2, 7.2 Hz, 1H), 2.89 (dddd, J = 11.9, 8.9, 6.9, 3.0 Hz, 1H), 2.25 (dddd, J = 12.1, 9.6, 7.8, 5.8 Hz, 1H) | 419.3 |
| 124 | 212 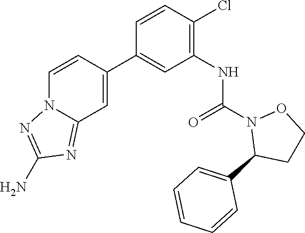 [SMILES: Nc1nc2cc(-c3ccc(Cl)c(NC(=O)N4OCC[C@H]4c4ccccc4)c3)ccn2n1] | (S)-N-(5-(2-amino-[1,2,4]triazolo[1,5- a]pyridin-7-yl)-2-chlorophenyl)-3- phenylisoxazolidine-2-carboxamide | ¹H NMR (400 MHz, DMSO-d₆) δ = 9.00 (s, 1H), 8.60 (dd, J = 6.9, 0.8 Hz, 1H), 8.25 (t, J = 1.3 Hz, 1H), 7.66-7.57 (m, 3H), 7.44-7.33 (m, 4H), 7.31-7.25 (m, 1H), 7.15 (dd, J = 7.0, 2.0 Hz, 1H), 6.07 (s, 2H), 5.41 (dd, J = 8.6, 6.0 Hz, 1H), 4.32 (td, J = 7.9, 3.0 Hz, 1H), 4.07-3.97 (m, 1H), 2.97-2.86 (m, 1H), 2.33-2.21 (m, 1H) | 435.2 |
| 125 | 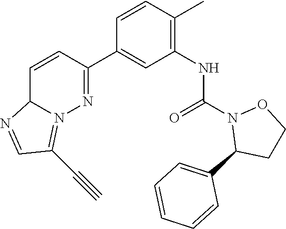 | (S)-N-(5-(3-ethynylimidazo[1,2- b]pyridazin-6-yl)-2-methylphenyl)-3- phenylisoxazolidine-2-carboxamide | ¹H NMR (400 MHz, DMSO-d₆) δ = 9.10 (s, 1H), 8.27 (d, J = 9.6 Hz, 1H), 8.11 (d, J = 5.0 Hz, 2H), 7.90-7.82 (m, 2H), 7.47-7.33 (m, 5H), 7.31-7.24 (m, 1H), 5.43 (dd, J = 8.6, 5.6 Hz, 1H), 4.99 (s, 1H), 4.29 (td, J = 7.9, 3.2 Hz, 1H), 4.05-3.96 (m, 1H), 2.93-2.85 (m, 1H), 2.32 (s, 4H) | 424.2 |
| 126 | 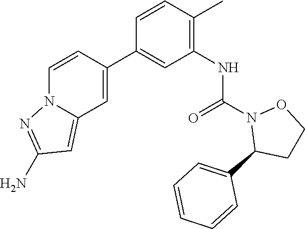 [SMILES: Cc1ccc(-c2ccn3nc(N)cc3c2)cc1NC(=O)N1OCC[C@H]1c1ccccc1] | (S)-N-(5-(2-aminopyrazolo[1,5-a]pyridin-5- yl)-2-methylphenyl)-3-phenylisoxazolidine- 2-carboxamide | ¹H NMR (400 MHz, DMSO-d₆) δ = 8.93 (s, 1H), 8.29 (d, J = 7.1 Hz, 1H), 7.77 (d, J = 2.0 Hz, 1H), 7.53-7.49 (m, 1H), 7.46 (dd, J = 7.9, 2.0 Hz, 1H), 7.43-7.34 (m, 4H), 7.33-7.25 (m, 2H), 6.79 (dd, J = 7.2, 2.1 Hz, 1H), 5.67 (d, J = 0.8 Hz, 1H), 5.42 (dd, J = 8.6, 5.7 Hz, 1H), 5.29 (s, 2H), 4.28 (td, J = 7.9, 3.2 Hz, 1H), 3.99 (dt, J = 9.2, 7.4 Hz, 1H), 2.93-2.83 (m, 1H), 2.30-2.20 (m, 4H) | 414.3 |
| 127 | 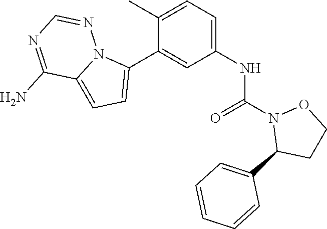 [SMILES: Cc1ccc(NC(=O)N2OCC[C@H]2c2ccccc2)cc1-c1ccc2c(N)ncnn12] | (S)-N-(3-(4-aminopyrrolo[2,1- f][1,2,4]triazin-7-yl)-4-methylphenyl)-3- phenylisoxazolidine-2-carboxamide | ¹H NMR (400 MHz, DMSO-d₆) δ = 9.41 (s, 1H), 7.79 (s, 3H), 7.65 (d, J = 2.3 Hz, 1H), 7.59 (dd, J = 8.3, 2.4 Hz, 1H), 7.41-7.32 (m, 4H), 7.31-7.24 (m, 1H), 7.21 (d, J = 8.4 Hz, 1H), 7.00 (d, J = 4.4 Hz, 1H), 6.64 (d, J = 4.4 Hz, 1H), 5.39 (dd, J = 8.6, 5.8 Hz, 1H), 4.23 (td, J = 7.9, 3.1 Hz, 1H), 3.87 (dt, J = 9.1, 7.7 Hz, 1H), 2.84 (dtd, J = 11.7, 8.2, 3.0 Hz, 1H), 2.26-2.16 (m, 1H), 2.07 (s, 3H) | 415.3 |
| 128 | 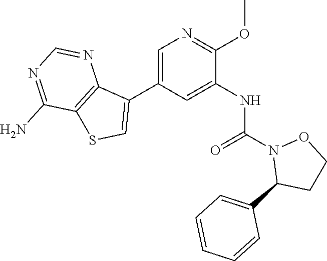 [SMILES: COc1ncc(-c2csc3c(N)ncnc23)cc1NC(=O)N1OCC[C@H]1c1ccccc1] | (S)-N-(5-(4-aminothieno[3,2-d]pyrimidine- 7-yl)-2-methoxypyridin-3-yl)-3- phenylisoxazolidine-2-carboxamide | | 449.2 |
| 129 | 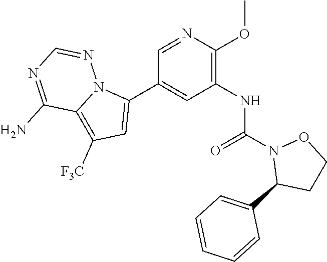 [SMILES: COc1ncc(-c2cc(C(F)(F)F)c3c(N)ncnn23)cc1NC(=O)N1OCC[C@H]1c1ccccc1] | (S)-N-(5-(4-amino-5- (trifluoromethyl)pyrrolo[2,1-f][1,2,4]triazin- 7-yl)-2-methoxypyridin-3-yl)-3- phenylisoxazolidine-2-carboxamide | ¹H NMR (400 MHz, DMSO-d₆) δ = 8.90 (d, J = 2.2 Hz, 1H), 8.53 (s, 1H), 8.47 (d, J = 2.2 Hz, 1H), 8.14 (s, 1H), 7.46 (s, 1H), 7.42-7.34 (m, 4H), 7.28 (ddt, J = 8.5, 6.0, 2.2 Hz, 1H), 5.40 (dd, J = 8.5, 6.2 Hz, 1H), 4.32 (td, J = 7.8, 2.9 Hz, 1H), 4.04 (s, 3H), 3.98 (ddd, J = 9.5, 8.0, 6.8 Hz, 1H), 2.91 (dddt, J = 12.0, 8.9, 6.9, 2.9 Hz, 1H), 2.30-2.21 (m, 1H) | 500.2 |
| 130 | 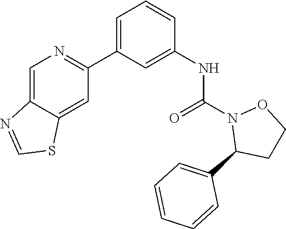 [SMILES: O=C(Nc1cccc(-c2cc3scnc3cn2)c1)N1OCC[C@H]1c1ccccc1] | (S)-N-(2-methyl-5-(thiazolo[4,5-c]pyridin- 6-yl)phenyl)-3-phenylisoxazolidine-2- carboxamide | ¹H NMR (400 MHz, DMSO-d₆) δ = 9.49 (s, 1H), 9.40 (s, 1H), 8.99 (s, 1H), 8.79 (s, 1H), 8.25 (d, J = 1.9 Hz, 1H), 7.90 (dd, J = 7.9, 2.0 Hz, 1H), 7.43-7.36 (m, 5H), 7.31- 7.25 (m, 1H), 5.43 (dd, J = 8.6, 5.7 Hz, 1H), 4.29 (td, J = 7.9, 3.2 Hz, 1H), 4.00 (dt, J = 9.3, 7.5 Hz, 1H), 2.93-2.85 (m, 1H), 2.31-2.23 (m, 4H) | 417.2 |
| 131 | 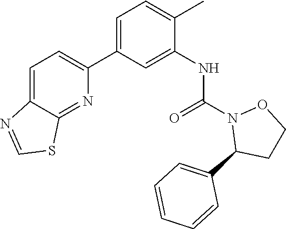 [SMILES: Cc1ccc(-c2ccc3ncsc3n2)cc1NC(=O)N1OCC[C@H]1c1ccccc1] | (S)-N-(2-methyl-5-(thiazolo[5,4-b]pyridin- 5-yl)phenyl)-3-phenylisoxazolidine-2- carboxamide | ¹H NMR (400 MHz, DMSO-d₆) δ = 9.53 (s, 1H), 9.01 (s, 1H), 8.52 (d, J = 8.6 Hz, 1H), 8.24 (d, J = 1.9 Hz, 1H), 8.14 (d, J = 8.6 Hz, 1H), 7.91 (dd, J = 7.9, 2.0 Hz, 1H), 7.43- 7.35 (m, 5H), 7.31-7.25 (m, 1H), 5.43 (dd, J = 8.6, 5.7 Hz, 1H), 4.29 (td, J = 8.0, 3.2 Hz, 1H), 4.05-3.97 (m, 1H), 2.89 (dq, J = 8.6, 4.8, 4.1 Hz, 1H), 2.30 (s, 4H) | 417.2 |
| 132 | 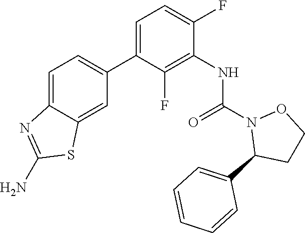 [SMILES: Nc1nc2ccc(-c3ccc(F)c(NC(=O)N4OCC[C@H]4c4ccccc4)c3F)cc2s1] | (S)-N-(3-(2-aminobenzo[d]thiazol-6-yl)- 2,6-difluorophenyl)-3-phenylisoxazolidine- 2-carboxamide | ¹H NMR (400 MHz, DMSO-d₆) δ = 9.25 (s, 1H), 8.12 (s, 2H), 7.88 (s, 1H), 7.51-7.43 (m, 2H), 7.42-7.34 (m, 5H), 7.30-7.22 (m, 2H), 5.37 (dd, J = 8.7, 5.7 Hz, 1H), 4.29 (td, J = 7.9, 3.1 Hz, 1H), 3.92-3.83 (m, 1H), 2.92-2.83 (m, 1H), 2.30-2.19 (m, 1H) | 453.2 |
| 133 | 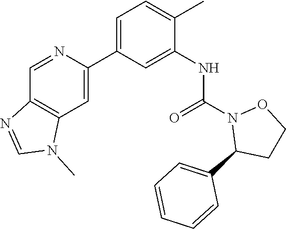 [SMILES: Cc1ccc(-c2cc3c(cn2)ncn3C)cc1NC(=O)N1OCC[C@H]1c1ccccc1] | (S)-N-(2-methyl-5-(1-methyl-1H- imidazo[4,5-c]pyridin-6-yl)phenyl)-3- phenylisoxazolidine-2-carboxamide | ¹H NMR (400 MHz, DMSO-d₆) δ = 9.01-8.95 (m, 2H), 8.33 (s, 1H), 8.21 (d, J = 1.9 Hz, 1H), 8.17 (d, J = 1.1 Hz, 1H), 7.92 (dd, J = 8.0, 1.9 Hz, 1H), 7.45-7.31 (m, 5H), 7.31-7.23 (m, 1H), 5.43 (dd, J = 8.6, 5.7 Hz, 1H), 4.28 (td, J = 8.0, 3.2 Hz, 1H), 4.00 (dt, J = 9.3, 7.5 Hz, 1H), 3.92 (s, 3H), 2.89 (dtd, J = 11.7, 8.3, 7.8, 3.2 Hz, 1H), 2.28 (s, 4H) | 414.3 |
| 134 | 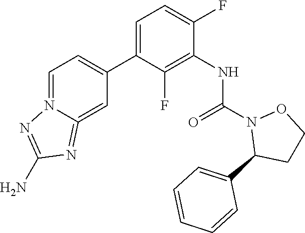 | (S)-N-(3-(2-amino-[1,2,4]triazolo[1,5- a]pyridin-7-yl)-2,6-difluorophenyl)-3- phenylisoxazolidine-2-carboxamide | ¹H NMR (400 MHz, DMSO-d₆) δ = 9.31 (s, 1H), 8.66 (d, J = 6.9 Hz, 1H), 7.64 (td, J = 8.6, 5.9 Hz, 1H), 7.52 (s, 1H), 7.42-7.23 (m, 6H), 7.08 (d, J = 6.9 Hz, 1H), 5.37 (dd, J = 8.7, 5.7 Hz, 1H), 4.30 (td, J = 8.0, 3.1 Hz, 1H), 3.89 (q, J = 8.0 Hz, 1H), 2.88 (dtd, J = 11.5, 7.9, 3.0 Hz, 1H), 2.24 (ddd, J = 13.3, 10.7, 6.8 Hz, 1H) | 437.2 |
| 135 | 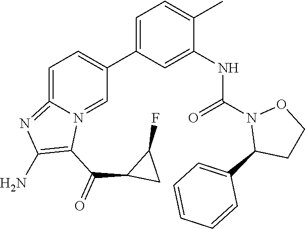 | (S)-N-(5-(2-amino-3-((1S,2S)-2- fluorocyclopropane-1- carbonyl)imidazo[1,2-a]pyridin-6-yl)-2- methylphenyl)-3-phenylisoxazolidine-2- carboxamide | ¹H NMR (400 MHz, DMSO-d₆) δ = 9.84 (s, 1H), 8.98 (s, 1H), 7.79 (dd, J = 9.1, 2.0 Hz, 1H), 7.71 (d, J = 1.9 Hz, 1H), 7.47-7.32 (m, 7H), 7.30-7.24 (m, 1H), 6.66 (s, 2H), 5.43 (dd, J = 8.6, 5.6 Hz, 1H), 5.29-5.07 (m, 1H), 4.27 (td, J = 7.9, 3.2 Hz, 1H), 4.00 (dt, J = 9.0, 7.4 Hz, 1H), 2.94- 2.83 (m, 1H), 2.60 (d, J = 8.0 Hz, 1H), 2.28 (s, 4H), 1.88 (dtd, J = 23.4, 6.9, 3.3 Hz, 1H), 1.12 (ddt, J = 12.4, 9.2, 6.2 Hz, 1H) | 500.3 |
| 136 | 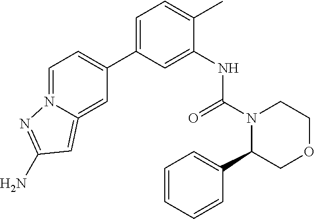 | (R)-N-(5-(2-aminopyrazolo[1,5-a]pyridin-5- yl)-2-methylphenyl)-3-phenylmorpholine-4- carboxamide | ¹H NMR (400 MHz, DMSO-d₆) δ = 8.29 (d, J = 7.1 Hz, 1H), 8.24 (s, 1H), 7.56 (d, J = 2.0 Hz, 1H), 7.49 (dd, J = 2.2, 0.9 Hz, 1H), 7.45-7.36 (m, 5H), 7.31-7.23 (m, 2H), 6.78 (dd, J = 7.2, 2.1 Hz, 1H), 5.68 (d, J = 0.8 Hz, 1H), 5.28 (d, J = 9.9 Hz, 3H), 4.38 (d, J = 11.9 Hz, 1H), 3.86 (td, J = 12.0, 11.5, 3.7 Hz, 3H), 3.56 (td, J = 11.6, 3.1 Hz, 1H), 3.19 (td, J = 12.9, 3.7 Hz, 1H), 2.13 (s, 3H) | 428.3 |
| 137 | 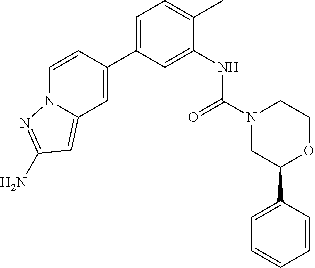 [SMILES: Cc1ccc(-c2ccn3nc(N)cc3c2)cc1NC(=O)N1CCO[C@@H](c2ccccc2)C1] | (S)-N-(5-(2-aminopyrazolo[1,5-a]pyridin-5- yl)-2-methylphenyl)-2-phenylmorpholine-4- carboxamide | ¹H NMR (400 MHz, DMSO-d₆) δ = 8.31-8.23 (m, 2H), 7.58 (d, J = 2.0 Hz, 1H), 7.50 (dd, J = 2.1, 0.9 Hz, 1H), 7.46- 7.36 (m, 5H), 7.35-7.30 (m, 1H), 7.28 (d, J = 8.0 Hz, 1H), 6.79 (dd, J = 7.2, 2.1 Hz, 1H), 5.67 (d, J = 0.8 Hz, 1H), 5.29 (s, 2H), 4.50 (dd, J = 10.6, 2.6 Hz, 1H), 4.20- 4.12 (m, 1H), 4.08-3.99 (m, 2H), 3.67 (td, J = 11.7, 2.8 Hz, 1H), 3.09-3.00 (m, 1H), 2.85 (dd, J = 13.1, 10.6 Hz, 1H), 2.22 (s, 3H) | 428.3 |
| 138 | 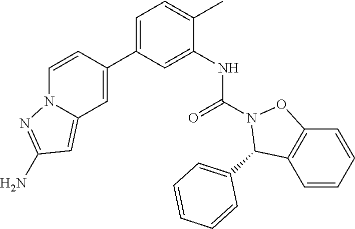 [SMILES: Cc1ccc(-c2ccn3nc(N)cc3c2)cc1NC(=O)N1Oc2ccccc2[C@@H]1c1ccccc1] | (S)-N-(5-(2-aminopyrazolo[1,5-a]pyridin-5- yl)-2-methylphenyl)-3- phenylbenzo[d]isooxazole-2(3H)- carboxamide | ¹H NMR (400 MHz, DMSO-d₆) δ = 9.37 (s, 1H), 8.27 (d, J = 7.1 Hz, 1H), 7.65 (d, J = 2.0 Hz, 1H), 7.51 (dd, J = 8.0, 2.0 Hz, 2H), 7.45-7.28 (m, 8H), 7.16 (d, J = 8.0 Hz, 1H), 7.09 (td, J = 7.5, 0.9 Hz, 1H), 6.80 (dd, J = 7.2, 2.1 Hz, 1H), 6.63 (s, 1H), 5.66 (d, J = 0.8 Hz, 1H), 5.29 (s, 2H), 2.17 (s, 3H) | 462.3 |
| 139 | 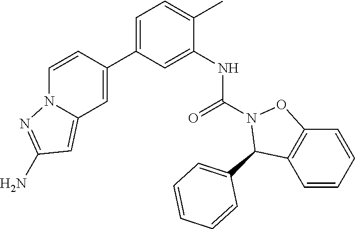 [SMILES: Cc1ccc(-c2ccn3nc(N)cc3c2)cc1NC(=O)N1Oc2ccccc2[C@H]1c1ccccc1] | (R)-N-(5-(2-aminopyrazolo[1,5-a]pyridin-5- yl)-2-methylphenyl)-3- phenylbenzo[d]isoxazole-2(3H)- carboxamide | ¹H NMR (400 MHz, DMSO-d₆) δ = 9.37 (s, 1H), 8.27 (d, J = 7.2 Hz, 1H), 7.65 (d, J = 2.0 Hz, 1H), 7.51 (dd, J = 7.9, 2.0 Hz, 2H), 7.45-7.27 (m, 8H), 7.16 (d, J = 8.0 Hz, 1H), 7.12-7.07 (m, 1H), 6.80 (dd, J = 7.2, 2.1 Hz, 1H), 6.63 (s, 1H), 5.66 (s, 1H), 5.29 (s, 2H), 2.17 (s, 3H) | 462.3 |
| 140 | 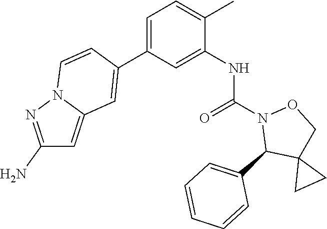 [SMILES: Cc1ccc(-c2ccn3nc(N)cc3c2)cc1NC(=O)N1OCC2(CC2)[C@H]1c1ccccc1] | (R)-N-(5-(2-aminopyrazolo[1,5-a]pyridin-5- yl)-2-methylphenyl)-7-phenyl-5-oxa-6- azaspiro[2.4]heptane-6-carboxamide | ¹H NMR (400 MHz, DMSO-d₆) δ = 9.04 (s, 1H), 8.29 (d, J = 7.1 Hz, 1H), 7.74 (d, J = 2.0 Hz, 1H), 7.53-7.45 (m, 2H), 7.38-7.26 (m, 6H), 6.79 (dd, J = 7.2, 2.1 Hz, 1H), 5.67 (d, J = 0.8 Hz, 1H), 5.29 (s, 2H), 5.13 (s, 1H), 4.18- 4.04 (m, 2H), 2.28 (s, 3H), 0.92 (ddd, J = 10.0, 6.1, 4.3 Hz, 1H), 0.81 (ddd, J = 10.3, 6.1, 4.4 Hz, 1H), 0.77-0.70 (m, 1H), 0.26 (dt, J = 10.5, 5.3 Hz, 1H) | 440.3 |
| 141 | 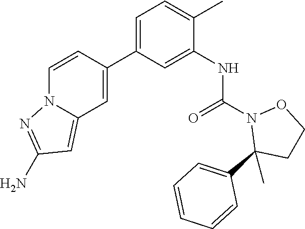 [SMILES: Cc1ccc(-c2ccn3nc(N)cc3c2)cc1NC(=O)N1OCC[C@@]1(C)c1ccccc1] | (S)-N-(5-(2-aminopyrazolo[1,5-a]pyridin-5- yl)-2-methylphenyl)-3-methyl-3- phenylisoxazolidine-2-carboxamide | ¹H NMR (400 MHz, DMSO-d₆) δ = 8.59 (s, 1H), 8.30 (d, J = 7.1 Hz, 1H), 7.66-7.57 (m, 3H), 7.48-7.36 (m, 4H), 7.34-7.26 (m, 2H), 6.75 (dd, J = 7.2, 2.1 Hz, 1H), 5.69 (d, J = 0.8 Hz, 1H), 5.30 (s, 2H), 3.90 (ddd, J = 10.4, 8.1, 6.2 Hz, 1H), 3.60 (ddd, J = 10.3, 8.2, 6.1 Hz, 1H), 2.61-2.51 (m, 2H), 2.18 (s, 3H), 1.62 (s, 3H) | 428.3 |
| 142 | 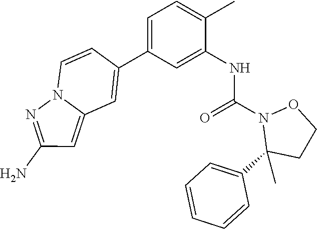 [SMILES: Cc1ccc(-c2ccn3nc(N)cc3c2)cc1NC(=O)N1OCC[C@]1(C)c1ccccc1] | (R)-N-(5-(2-aminopyrazolo[1,5-a]pyridin-5- yl)-2-methylphenyl)-3-methyl-3- phenylisoxazolidine-2-carboxamide | ¹H NMR (400 MHz, DMSO-d₆) δ = 8.59 (s, 1H), 8.30 (d, J = 7.0 Hz, 1H), 7.65-7.57 (m, 3H), 7.49-7.36 (m, 4H), 7.34-7.25 (m, 2H), 6.75 (dd, J = 7.2, 2.1 Hz, 1H), 5.69 (d, J = 0.8 Hz, 1H), 5.30 (s, 2H), 3.90 (ddd, J = 10.5, 8.1, 6.3 Hz, 1H), 3.61 (ddd, J = 10.4, 8.2, 6.1 Hz, 1H), 2.61-2.51 (m, 2H), 2.18 (s, 3H), 1.62 (s, 3H) | 428.3 |
| 143 | 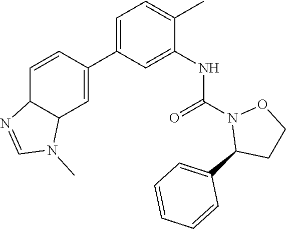 [SMILES: Cc1ccc(C2=CC3C(C=C2)N=CN3C)cc1NC(=O)N1OCC[C@H]1c1ccccc1] | (S)-N-(2-methyl-5-(3-methyl-3H- imidazo[4,5-b]pyridin-5-yl)phenyl)-3- phenylisoxazolidine-2-carboxamide | ¹H NMR (400 MHz, DMSO-d₆) δ = 9.05 (s, 1H), 8.43 (s, 1H), 8.17 (d, J = 1.9 Hz, 1H), 8.12 (d, J = 8.4 Hz, 1H), 7.91 (dd, J = 7.9, 1.9 Hz, 1H), 7.82 (d, J = 8.5 Hz, 1H), 7.39 (ddd, J = 19.7, 8.1, 5.7 Hz, 5H), 7.31-7.26 (m, 1H), 5.44 (dd, J = 8.7, 5.6 Hz, 1H), 4.29 (td, J = 8.0, 3.2 Hz, 1H), 4.01 (dt, J = 9.4, 7.5 Hz, 1H), 3.90 (s, 3H), 2.90 (dtd, J = 11.4, 7.9, 3.1 Hz, 1H), 2.29 (s, 3H), 2.27-2.21 (m, 1H) | 414.3 |
| 144 | 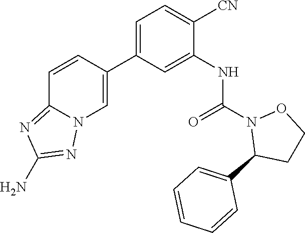 [SMILES: N#Cc1ccc(-c2ccc3nc(N)nn3c2)cc1NC(=O)N1OCC[C@H]1c1ccccc1] | (S)-N-(5-(2-amino-[1,2,4]triazolo[1,5- a]pyridin-6-yl)-2-cyanophenyl)-3- phenylisoxazolidine-2-carboxamide | ¹H NMR (400 MHz, DMSO-d₆) δ = 9.71 (s, 1H), 9.02 (dd, J = 2.0, 0.9 Hz, 1H), 8.00 (d, J = 1.8 Hz, 1H), 7.90 (d, J = 8.2 Hz, 1H), 7.78 (ddd, J = 20.8, 8.7, 1. 9 Hz, 2H), 7.49-7.45 (m, 1H), 7.43-7.35 (m, 4H), 7.32-7.26 (m, 1H), 6.19 (s, 2H), 5.43 (dd, J = 8.6, 5.8 Hz, 1H), 4.32 (td, J = 7.8, 3.0 Hz, 1H), 4.05-3.96 (m, 1H), 2.97-2.86 (m, 1H), 2.31-2.21 (m, 1H) | 426.3 |
| 145 | 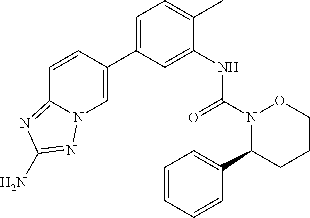 [SMILES: Cc1ccc(-c2ccc3nc(N)nn3c2)cc1NC(=O)N1OCCC[C@H]1c1ccccc1] | (S)-N-(5-(2-amino-[1,2,4]triazolo[1,5- a]pyridin-6-yl)-2-methylphenyl)-3-phenyl- 1,2-oxazinane-2-carboxamide | | 429.2 |
| 146 | 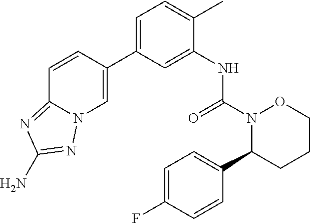 [SMILES: Cc1ccc(-c2ccc3nc(N)nn3c2)cc1NC(=O)N1OCCC[C@H]1c1ccc(F)cc1] | (S)-N-(5-(2-amino-[1,2,4]triazolo[1,5- a]pyridin-6-yl)-2-methylphenyl)-3-(4- fluorophenyl)-1,2-oxazinane-2-carboxamide | | 447.2 |
| 147 | 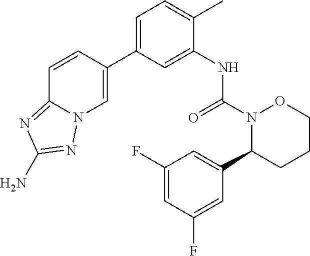 [SMILES: Cc1ccc(-c2ccc3nc(N)nn3c2)cc1NC(=O)N1OCCC[C@H]1c1cc(F)cc(F)c1] | (S)-N-(5-(2-amino-[1,2,4]triazolo[1,5- a]pyridin-6-yl)-2-methylphenyl)-3-(3,5- difluorophenyl)-1,2-oxazinane-2- carboxamide | | 465.2 |
| 148 | 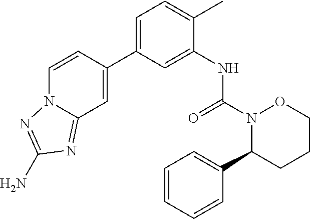 [SMILES: Cc1ccc(-c2ccn3nc(N)nc3c2)cc1NC(=O)N1OCCC[C@H]1c1ccccc1] | (S)-N-(5-(2-amino-[1,2,4]triazolo[1,5- a]pyridin-7-yl)-2-methylphenyl)-3-phenyl- 1,2-oxazinane-2-carboxamide | | 429.2 |
| 149 | 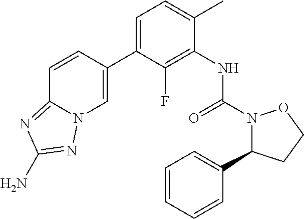 [SMILES: Cc1ccc(-c2ccc3nc(N)nn3c2)c(F)c1NC(=O)N1OCC[C@H]1c1ccccc1] | (S)-N-(3-(2-amino-[1,2,4]triazolo[1,5- a]pyridin-6-yl)-2-fluoro-6-methylphenyl)-3- phenylisoxazolidine-2-carboxamide | | 433.2 |
| 150 | 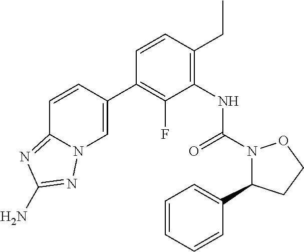 [SMILES: CCc1ccc(-c2ccc3nc(N)nn3c2)c(F)c1NC(=O)N1OCC[C@H]1c1ccccc1] | (S)-N-(3-(2-amino-[1,2,4]triazolo[1,5- a]pyridin-6-yl)-6-ethyl-2-fluorophenyl)-3- phenylisoxazolidine-2-carboxamide | | 447.2 |
| 151 | 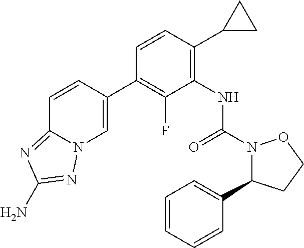 [SMILES: Nc1nc2ccc(-c3ccc(C4CC4)c(NC(=O)N4OCC[C@H]4c4ccccc4)c3F)cn2n1] | (S)-N-(3-(2-amino-[1,2,4]triazolo[1,5- a]pyridin-6-yl)-6-cyclopropyl-2- fluorophenyl)-3-phenylisoxazolidine-2- carboxamide | | 459.2 |
| 152 | 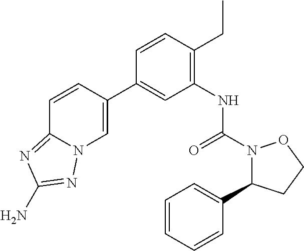 [SMILES: CCc1ccc(-c2ccc3nc(N)nn3c2)cc1NC(=O)N1OCC[C@H]1c1ccccc1] | (S)-N-(5-(2-amino-[1,2,4]triazolo[1,5- a]pyridin-6-yl)-2-ethylphenyl)-3- phenylisoxazolidine-2-carboxamide | | 429.2 |
| 153 | 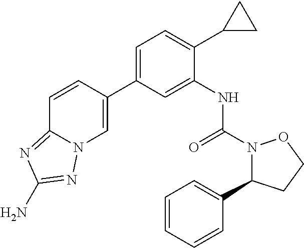 [SMILES: Nc1nc2ccc(-c3ccc(C4CC4)c(NC(=O)N4OCC[C@H]4c4ccccc4)c3)cn2n1] | (S)-N-(5-(2-amino-[1,2,4]triazolo[1,5- a]pyridin-6-yl)-2-cyclopropylphenyl)-3- phenylisoxazolidine-2-carboxamide | | 441.2 |
| 154 | 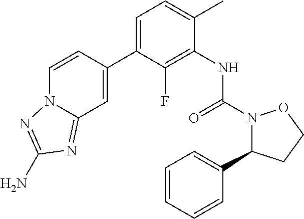 [SMILES: Cc1ccc(-c2ccn3nc(N)nc3c2)c(F)c1NC(=O)N1OCC[C@H]1c1ccccc1] | (S)-N-(3-(2-amino-[1,2,4]triazolo[1,5- a]pyridin-7-yl)-2-fluoro-6-methylphenyl)-3- phenylisoxazolidine-2-carboxamide | | 433.2 |
| 155 | 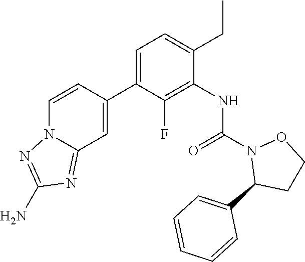 [SMILES: CCc1ccc(-c2ccn3nc(N)nc3c2)c(F)c1NC(=O)N1OCC[C@H]1c1ccccc1] | (S)-N-(3-(2-amino-[1,2,4]triazolo[1,5- a]pyridin-7-yl)-6-ethyl-2-fluorophenyl)-3- phenylisoxazolidine-2-carboxamide | | 447.2 |
| 156 | 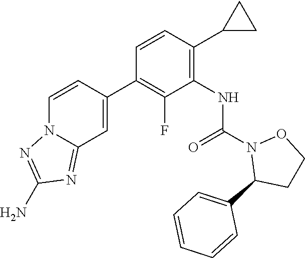 [SMILES: Nc1nc2cc(-c3ccc(C4CC4)c(NC(=O)N4OCC[C@H]4c4ccccc4)c3F)ccn2n1] | (S)-N-(3-(2-amino-[1,2,4]triazolo[1,5- a]pyridin-7-yl)-6-cyclopropyl-2- fluorophenyl)-3-phenylisoxazolidine-2- carboxamide | | 459.2 |
| 157 | 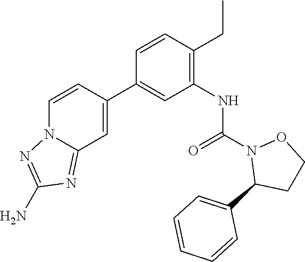 [SMILES: CCc1ccc(-c2ccn3nc(N)nc3c2)cc1NC(=O)N1OCC[C@H]1c1ccccc1] | (S)-N-(5-(2-amino-[1,2,4]triazolo[1,5- a]pyridin-7-yl)-2-ethylphenyl)-3- phenylisoxazolidine-2-carboxamide | | 429.2 |
| 158 | 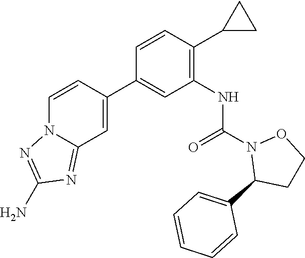 [SMILES: Nc1nc2cc(-c3ccc(C4CC4)c(NC(=O)N4OCC[C@H]4c4ccccc4)c3)ccn2n1] | (S)-N-(5-(2-amino-[1,2,4]triazolo[1,5- a]pyridin-7-yl)-2-cyclopropylphenyl)-3- phenylisoxazolidine-2-carboxamide | | 441.2 |

### <Experimental Example 1> Evaluation of RIPK1 Inhibitory Activity of Compound According to the Present Invention

To evaluate the RIPK1 inhibitory activity of a compound according to the present invention, the following experiment was performed. Specifically, for Examples 3 and 30 selected from the compounds of Examples of the present invention, enzyme (kinase) selectivity was measured by DiscoverX, and the experiment was conducted using a panel for scanMAX™ kinase analysis. Here, the enzyme was treated with a drug having a concentration of 1 μM in DMSO, and a control percentage (% control) was determined in the same manner as Formula 1 below. The results are shown in Table 3 below.

$$( Compound of Example - Positive control ) / ( Negative control - Positive control ) \times 100 \qquad [ Formula 1 ]$$

Here, the positive control refers to a compound showing a control percentage of 0%, and the negative control is DMSO showing a control percentage of 100%. In addition, in terms of the enzyme selectivity of the present invention, when the control percentage with respect to an enzyme is <35% (i.e., less than 35%), it was determined that the compound has activity against the corresponding enzyme.

TABLE 3

| Kinase | Example 3 | Example 30 |
|---|---|---|
| RIPK1 | 1% | 0% |

As confirmed in Table 3, since compounds of Example according to the present invention have a smaller value than the control percentage (35%) with respect to RIPK1, it can be seen that the compounds have activity against RIPK1. This suggests that there is a useful effect when a compound of Example according to the present invention is used for an RIPK1-related disease, and therefore, it can be effectively used as a composition for treating or preventing an RIPK1-related disease.

### <Experimental Example 2> Evaluation of Cell Necrosis Inhibitory Activity of Compound According to the Present Invention Under Necroptosis-Inducing Conditions in HT-29 Cells

To evaluate the cell necrosis inhibitory activity of the compound according to the present invention, an experiment was conducted as follows.

To evaluate the efficacy of inhibiting cell necrosis under TNF-α-mediated cell death-inducing conditions, a HT-29 cell line (Korean Cell Line Bank Accession No. #30038) was cultured in RPMI-1640 (HyClone #SH3027.01) medium containing 10% fetal bovine serum (FBS) and 1% penicillin/streptomycin to analyze cell viability. More specifically, for testing, the cell line was seeded in a 96-well flat bottom plate (Corning #3903) at a concentration of 10,000 cells/well and incubated at 37° C. under a 5% CO₂ condition for 24 hours. Each well was treated with 100 ng/mL TNF-α, 25 μM pan-caspase inhibitor (Z-VAD-FMK, Selleckchem #S8102), and 10 UM Smac mimetic LCL161 (Selleckchem #S7009) and allowed to react for 10 minutes. The compounds of Examples were treated at 11 concentrations (the highest concentration being 0.1 μM or 1 μM) with a 3-fold concentration gradient, and as a control, dimethyl sulfoxide (DMSO) was treated at a concentration of 0.05% (v/v), which is the same as those when the compounds were treated. The treated cells were incubated for 24 hours. To confirm the degree of cell viability, 100 μL of Cell Titer-Glo (Promega #G7573) was added to the medium of the cultured cells and cultured for additional 10 minutes, and then luminescence was measured using a microplate reader. Based on the fluorescence of control cells that were not treated with the compound, the degree of cell necrosis inhibitory activity was calculated according to the treatment concentration of each compound. Here, the concentration at which the cell necrosis inhibitory activity was 50% was determined as an EC₅₀ (nM) value and calculated using Prism (ver. 8.4.3 #GraphPad) software. The results are summarized in Table 4.

TABLE 4

| | HT-29 |
|---|---|
| Example | EC₅₀(nM) |
| 1 | 0.9 |
| 2 | 0.2 |
| 3 | 0.6 |
| 4 | 0.2 |
| 5 | 2.9 |
| 6 | 0.9 |
| 7 | 40.0 |
| 8 | 0.4 |
| 10 | 0.3 |
| 11 | 0.9 |
| 12 | 13.7 |
| 13 | 2.6 |
| 14 | 0.7 |
| 15 | 23.9 |
| 16 | 4.9 |
| 17 | 0.1 |
| 18 | 0.3 |
| 19 | 1.0 |
| 20 | 0.3 |
| 21 | 0.4 |
| 22 | 0.3 |
| 23 | 8.1 |
| 24 | 0.4 |
| 25 | 0.2 |
| 26 | 0.7 |
| 27 | 1.0 |
| 28 | 0.2 |
| 29 | 0.1 |
| 30 | 3.8 |
| 31 | 4.6 |
| 32 | 0.6 |
| 33 | 1.2 |
| 34 | 0.5 |
| 35 | 3.2 |
| 36 | 0.6 |
| 37 | 1.1 |
| 38 | 1.6 |
| 39 | 0.9 |
| 41 | 1.0 |
| 42 | 0.6 |
| 43 | 7.3 |
| 44 | 4.1 |
| 45 | 4.6 |
| 46 | 19.4 |
| 47 | 0.5 |
| 48 | 0.4 |
| 49 | 1.3 |
| 50 | 1.3 |
| 51 | 23.3 |
| 52 | 2.8 |
| 53 | 0.5 |
| 54 | 0.4 |
| 55 | 0.6 |
| 56 | 0.7 |
| 57 | 4.4 |
| 58 | 3.9 |
| 59 | 0.7 |
| 60 | 0.4 |
| 61 | 0.2 |
| 62 | 0.2 |
| 63 | 0.2 |
| 64 | 0.2 |
| 65 | 0.2 |
| 66 | 0.4 |
| 67 | 0.4 |
| 68 | 3.9 |
| 69 | 0.4 |
| 70 | 5.2 |
| 91 | 0.9 |
| 92 | 0.6 |
| 93 | 0.2 |
| 94 | 0.3 |
| 95 | 7.5 |
| 96 | 1.9 |
| 97 | 1.1 |
| 98 | 0.3 |
| 99 | 8.2 |
| 100 | 4.6 |
| 101 | 6.2 |
| 102 | 0.9 |
| 103 | 15.6 |
| 105 | 43.8 |
| 106 | 5.3 |
| 107 | 23.2 |
| 108 | 29.8 |
| 109 | 1.0 |
| 110 | 4.0 |
| 112 | 3.3 |
| 113 | 2.4 |
| 114 | 1.9 |
| 115 | 1.7 |
| 116 | 1.0 |
| 117 | 2.4 |
| 118 | 4.3 |
| 120 | 1.0 |
| 121 | 11.7 |
| 122 | 20.4 |
| 123 | 0.3 |
| 124 | 0.2 |
| 125 | 1.4 |
| 126 | 0.4 |
| 128 | 38.2 |
| 129 | 3.6 |
| 130 | 1.4 |
| 131 | 4.0 |
| 132 | 8.1 |
| 133 | 6.5 |
| 134 | 13.0 |
| 135 | 0.2 |
| 136 | 5.4 |
| 137 | 49.2 |
| 139 | 2.2 |
| 140 | 1.7 |
| 143 | 5.0 |

1. A compound of Chemical Formula 1 below, an isomer thereof, a solvate thereof, a hydrate thereof, or a pharmaceutically acceptable salt thereof: 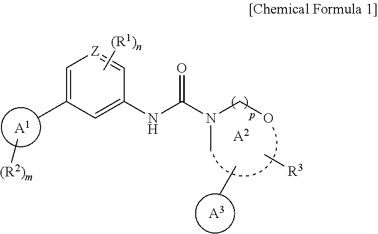
In Chemical Formula 1,
Z is N or CH,
ring A¹ is 5- to 12-membered heteroaryl,
ring A² is 5- to 10-membered heterocycloalkyl, 5- to 10-membered heterocycloalkyl-aryl bicyclic ring, or 5- to 10-membered heterocycloalkyl-heteroaryl bicyclic ring, wherein p is an integer from 0 to 2,
ring A³ is 4- to 12-membered aryl or 4- to 12-membered heteroaryl, and is unsubstituted or substituted with one or more substituents R⁴ selected from halogen, cyano, C₁₋₈ alkoxy, C₁₋₈ alkyl, C₁₋₈ haloalkyl, and C₁₋₈ alkylamino,
R¹ is hydrogen, halogen, C₁₋₈ alkyl, C₁₋₈ alkoxy, cyano, C₃₋₈ cycloalkyl, or C₃₋₈ heterocycloalkyl, and n is an integer from 1 to 3,
R² is halogen, C₁₋₈ alkyl, C₁₋₈ alkoxy, amine, cyano, C₁₋₈ haloalkyl, or C₂₋₈ alkynyl that is unsubstituted or substituted with a hydroxyl group, 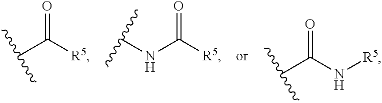
and m is an integer from 0 to 4,
R⁵ is C₁₋₈ alkyl, —C₁₋₈ alkyl-C₁₋₈ alkoxy, —C₁₋₈ alkyl-hydroxy, or C₃₋₈ cycloalkyl, where the C₃₋₈ cycloalkyl of R⁵ is unsubstituted or substituted with C₁₋₈ alkylamino or halogen, and
R³ is hydrogen or C₁₋₈ alkyl.

2. The compound of Chemical Formula 1, the isomer thereof, the solvate thereof, the hydrate thereof, or the pharmaceutically acceptable salt thereof of claim 1, wherein Z is N or CH,
ring A¹ is 8- to 10-membered heteroaryl that includes one or more hetero atoms selected from the group consisting of N, O, and S,
ring A² is 5- to 8-membered heterocycloalkyl or 8- to 10-membered heterocycloalkyl-aryl bicyclic ring, where p is an integer from 0 to 2,
ring A³ is 5- to 10-membered aryl or 4- to 8-membered heteroaryl including one or more hetero atoms selected from N, O, and S, and is unsubstituted or substituted with one or more substituents R⁴ selected from halogen, cyano, C₁₋₆ alkoxy, C₁₋₆ alkyl, C₁₋₆ haloalkyl, and C₁₋₆alkylamino,
R¹ is hydrogen, halogen, C₁₋₆ alkyl, C₁₋₆ alkoxy, cyano, or C₃₋₆ cycloalkyl, and n is an integer from 1 to 3,
R² is halogen, C₁₋₆ alkyl, C₁₋₆ alkoxy, amine, cyano, C₁₋₆ haloalkyl, or C₂₋₆ alkynyl that is unsubstituted or substituted with a hydroxyl group, 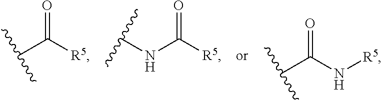
and m is an integer from 0 to 4,
R⁵ is C₁-6 alkyl, —C₁₋₆ alkyl-C₁₋₆ alkoxy, —C₁₋₆ alkyl-hydroxy, or C₃₋₆ cycloalkyl, where the C₃₋₆ cycloalkyl of R⁵ is unsubstituted or substituted with C₁₋₆ alkylamino or halogen, and
R³ is hydrogen or C₁₋₆ alkyl.

3. The compound of Chemical Formula 1, the isomer thereof, the solvate thereof, the hydrate thereof, or the pharmaceutically acceptable salt thereof of claim 1, wherein ring A¹ is imidazo[1,2-b]pyridazine, benzo[d]oxazole, benzo[d][1,2,3]triazole, benzo[d]thiazole, indazole, thiazolo[5,4-b]pyridine, triazolo[1,5-a]pyridine, pyrrolo[2,1-f][1,2,4]triazine, triazolo[4,3-b]pyridazine, imidazo[1,2-a]pyridine, thieno[3,2-d]pyrimidine, pyrazolo[1,5-a]pyridine, thiazolo[4,5-c]pyridine, imidazo[4,5-c]pyridine, or imidazo[4,5-b]pyridine.

4. The compound of Chemical Formula 1, the isomer thereof, the solvate thereof, the hydrate thereof, or the pharmaceutically acceptable salt thereof of claim 1, wherein ring A² is isoxazolidine, morpholine, benzo[d]isoxazolidine, oxa-azaspiro[2.4]heptane, or oxazinane.

5. The compound of Chemical Formula 1, the isomer thereof, the solvate thereof, the hydrate thereof, or the pharmaceutically acceptable salt thereof of claim 1, wherein, when R² is 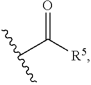
R⁵ is C₃₋₈ cycloalkyl that is unsubstituted or substituted with halogen,
when R² is 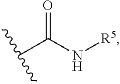
R⁵ is C₁₋₈ alkyl or C₃₋₈ cycloalkyl that is unsubstituted or substituted with C₁₋₈ alkylamino, and
when R² is 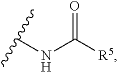
R⁵ may be C₃₋₈ cycloalkyl that is unsubstituted or substituted with C₁₋₈ alkyl, —C₁₋₈ alkyl-C₁₋₈ alkoxy, —C₁₋₈ alkyl-hydroxy, or halogen.

6. The compound of Chemical Formula 1, the isomer thereof, the solvate thereof, the hydrate thereof, or the pharmaceutically acceptable salt thereof of claim 1, wherein the compound represented by Chemical Formula 1 is any one of compounds 1 to 158 below:
<1> (S)—N-(3-(2-acetamidoimidazo[1,2-b]pyridazin-6-yl)phenyl)-3-phenylisoxazolidine-2-carboxamide; <2> (S)—N-(3-(2-(cyclopropanecarboxamido)imidazo[1,2-b]pyridazin-6-yl)phenyl)-3-phenylisoxazolidine-2-carboxamide; <3> (S)—N-(5-(2-acetamidoimidazo[1,2-b]pyridazin-6-yl)-2-methoxypyridin-3-yl)-3-phenylisoxazolidine-2-carboxamide; <4> (S)—N-(5-(2-(cyclopropanecarboxamido)imidazo[1,2-b]pyridazin-6-yl)-2-methoxypyridin-3-yl)-3-phenylisoxazolidine-2-carboxamide; <5> (S)—N-(5-(2-(2-hydroxyacetamido)imidazo[1,2-b]pyridazin-6-yl)-2-methoxypyridin-3-yl)-3-phenylisoxazolidine-2-carboxamide; <6> (S)—N-(2-methoxy-5-(2-(2-methoxyacetamido)imidazo[1,2-b]pyridazin-6-yl)pyridin-3-yl)-3-phenylisoxazolidine-2-carboxamide: <7> (S)—N-(5-(2-aminoimidazo[1,2-b]pyridazin-6-yl)-2-methoxypyridin-3-yl)-3-phenylisoxazolidine-2-carboxamide: <8> (S)—N-(5-(2-acetamidoimidazo[1,2-b]pyridazin-6-yl)-2-methoxyphenyl)-3-phenylisoxazolidine-2-carboxamide: <9> (S)—N-(5-(2-aminoimidazo[1,2-b]pyridazin-6-yl)-2-methoxyphenyl)-3-phenylisoxazolidine-2-carboxamide: <10> (S)—N-(5-(2-(cyclopropanecarboxamido)imidazo[1,2-b]pyridazin-6-yl)-2-methylpyridin-3-yl)-3-phenylisoxazolidine-2-carboxamide: <11> (S)—N-(5-(2-acetamidoimidazo[1,2-b]pyridazin-6-yl)-2-methylpyridin-3-yl)-3-phenylisoxazolidine-2-carboxamide; <12> (S)—N-(5-(1-isopropyl-1H-benzo[d][1,2,3]triazol-6-yl)-2-methoxypyridin-3-yl)-3-phenylisoxazolidine-2-carboxamide: <13> (S)—N-(5-(2-acetamidobenzo[d]oxazol-6-yl)-2-methoxypyridin-3-yl)-3-phenylisoxazolidine-2-carboxamide: <14> (S)—N-(5-(2-acetamidothiazolo[5,4-b]pyridin-5-yl)-2-methoxypyridin-3-yl)-3-phenylisoxazolidine-2-carboxamide; <15> (S)—N-(5-(1-isopropyl-1H-benzo[d][1,2,3]triazol-6-yl)-2-methoxyphenyl)-3-phenylisoxazolidine-2-carboxamide: <16> (S)—N-(5-(2-acetamidobenzo[d]oxazol-6-yl)-2-methoxyphenyl)-3-phenylisoxazolidine-2-carboxamide: <17> (S)—N-(3-(3-(methylcarbamoyl)-1H-indazol-6-yl)phenyl)-3-phenylisoxazolidine-2-carboxamide: <18> (S)—N-(6-methyl-5-(3-(methylcarbamoyl)-1H-indazol-6-yl)pyridin-3-yl)-3-phenylisoxazolidine-2-carboxamide; <19> (S)—N-(5-(3-(methylcarbamoyl)-1H-indazol-6-yl)pyridin-3-yl)-3-phenylisoxazolidine-2-carboxamide: <20> (S)—N-(3-(3-((4-(dimethylamino)cyclohexyl) carbamoyl)-1H-indazol-6-yl)phenyl)-3-phenylisoxazolidine-2-carboxamide; <21> (S)—N-(2-methyl-5-(3-(methylcarbamoyl)-1H-indazol-6-yl)pyridin-3-yl)-3-phenylisoxazolidine-2-carboxamide: <22> (S)—N-(2-methoxy-5-(3-(methylcarbamoyl)-1H-indazol-6-yl)pyridin-3-yl)-3-phenylisoxazolidine-2-carboxamide: <23> (S)—N-(6-methoxy-5-(3-(methylcarbamoyl)-1H-indazol-6-yl)pyridin-3-yl)-3-phenylisoxazolidine-2-carboxamide; <24> (S)—N-(2-methoxy-5-(3-(methylcarbamoyl)-1H-indazol-6-yl)phenyl)-3-phenylisoxazolidine-2-carboxamide: <25> (S)—N-(3-(2-(cyclopropanecarboxamido)benzo[d]thiazol-6-yl)phenyl)-3-phenylisoxazolidine-2-carboxamide: <26> (S)—N-(5-(2-acetamidobenzo[d]thiazol-6-yl)-2-methoxypyridin-3-yl)-3-phenylisoxazolidine-2-carboxamide; <27> (S)—N-(5-(2-acetamidobenzo[d]thiazol-6-yl)-2-methoxyphenyl)-3-phenylisoxazolidine-2-carboxamide: <28> (S)—N-(5-(2-acetamidobenzo[d]thiazol-6-yl)-2-methylpyridin-3-yl)-3-phenylisoxazolidine-2-carboxamide: <29> (S)—N-(5-(2-(cyclopropanecarboxamido)benzo[d]thiazol-6-yl)-2-methylpyridin-3-yl)-3-phenylisoxazolidine-2-carboxamide: <30> (S)—N-(5-(2-amino-[1,2,4]triazolo[1,5-a]pyridin-6-yl)-2-methoxypyridin-3-yl)-3-phenylisoxazolidine-2-carboxamide: <31> (S)—N-(5-(2-amino-[1,2,4]triazolo[1,5-a]pyridin-6-yl)-2-methoxyphenyl)-3-phenylisoxazolidine-2-carboxamide: <32> (S)—N-(5-(2-amino-[1,2,4]triazolo[1,5-a]pyridin-6-yl)-2-methylphenyl)-3-phenylisoxazolidine-2-carboxamide; <33> (S)—N-(5-(2-amino-[1,2,4]triazolo[1,5-a]pyridin-6-yl)-2-methylpyridin-3-yl)-3-phenylisoxazolidine-2-carboxamide: <34> (S)—N-(5-(2-amino-[1,2,4]triazolo[1,5-a]pyridin-6-yl)-4-fluoro-2-methylphenyl)-3-phenylisoxazolidine-2-carboxamide: <35> (S)—N-(5-(2-acetamido-[1,2,4]triazolo[1,5-a]pyridin-6-yl)-2-methylpyridin-3-yl)-3-phenylisoxazolidine-2-carboxamide: <36> (S)—N-(5-(2-(cyclopropanecarboxamido)-[1,2,4]triazolo[1,5-a]pyridin-6-yl)-2-methylpyridin-3-yl)-3-phenylisoxazolidine-2-carboxamide: <37> (S)—N-(5-(2-amino-[1,2,4]triazolo[1,5-a]pyridin-6-yl)-2-methylpyridin-3-yl)-3-(3-fluorophenyl)isoxazolidine-2-carboxamide; <38> (S)—N-(5-(2-amino-[1,2,4]triazolo[1,5-a]pyridin-6-yl)-2-methylpyridin-3-yl)-3-(3,5-difluorophenyl)isoxazolidine-2-carboxamide: <39> (S)—N-(5-(2-amino-[1,2,4]triazolo[1,5-a]pyridin-6-yl)-2-methylpyridin-3-yl)-3-(3-chlorophenyl)isoxazolidine-2-carboxamide: <40> (S)—N-(5-(2-amino-[1,2,4]triazolo[1,5-a]pyridin-6-yl)-2-methylpyridin-3-yl)-3-(pyridin-3-yl) isoxazolidine-2-carboxamide: <41> (S)—N-(5-(2-amino-[1,2,4]triazolo[1,5-a]pyridin-6-yl)-2-methylpyridin-3-yl)-3-(3-(trifluoromethyl)phenyl)isoxazolidine-2-carboxamide: <42> (S)—N-(5-(2-amino-[1,2,4]triazolo[1,5-a]pyridin-6-yl)-2-methylpyridin-3-yl)-3-(3-chloro-4-fluorophenyl)isoxazolidine-2-carboxamide: <43> (S)—N-(5-(2-amino-[1,2,4]triazolo[1,5-a]pyridin-6-yl)-2-methylpyridin-3-yl)-3-(3-fluoro-5-(trifluoromethyl)phenyl)isoxazolidine-2-carboxamide: <44> (S)—N-(5-(2-amino-[1,2,4]triazolo[1,5-a]pyridin-6-yl)-2-methylpyridin-3-yl)-3-(4-chloro-3-(trifluoromethyl)phenyl)isoxazolidine-2-carboxamide: <45> (S)—N-(5-(2-amino-[1,2,4]triazolo[1,5-a]pyridin-6-yl)-2-methylpyridin-3-yl)-3-(3-methoxyphenyl)isoxazolidine-2-carboxamide; <46> (S)—N-(5-(2-amino-[1,2,4]triazolo[1,5-a]pyridin-6-yl)-2-methylpyridin-3-yl)-3-(3-cyanophenyl)isoxazolidine-2-carboxamide: <47> (S)—N-(5-(2-amino-[1,2,4]triazolo[1,5-a]pyridin-7-yl)-2-methoxypyridin-3-yl)-3-phenylisoxazolidine-2-carboxamide: <48> (S)—N-(5-(2-(cyclopropanecarboxamido)-[1,2,4]triazolo[1,5-a]pyridin-7-yl)-2-methylpyridin-3-yl)-3-phenylisoxazolidine-2-carboxamide: <49> (S)—N-(5-(2-acetamido-[1,2,4]triazolo[1,5-a]pyridin-7-yl)-2-methylpyridin-3-yl)-3-phenylisoxazolidine-2-carboxamide: <50> (S)—N-(5-(2-amino-[1,2,4]triazolo[1,5-a]pyridin-7-yl)-2-methylpyridin-3-yl)-3-phenylisoxazolidine-2-carboxamide: <51> (S)—N-(5-(2-amino-[1,2,4]triazolo[1,5-a]pyridin-6-yl)-2-methylpyridin-3-yl)-3-(6-methylpyridin-3-yl) isoxazolidine-2-carboxamide: <52> (S)—N-(5-(2-amino-[1,2,4]triazolo[1,5-a]pyridin-6-yl)-2-methylpyridin-3-yl)-3-(3-chloro-5-fluorophenyl)isoxazolidine-2-carboxamide: <53> (S)—N-(5-(2-amino-[1,2,4]triazolo[1,5-a]pyridin-7-yl)-2-methoxypyridin-3-yl)-3-(3-fluorophenyl)isoxazolidine-2-carboxamide: <54> (S)—N-(5-(2-amino-[1,2,4]triazolo[1,5-a]pyridin-7-yl)-2-methoxypyridin-3-yl)-3-(3,5-difluorophenyl)isoxazolidine-2-carboxamide: <55> (S)—N-(5-(2-amino-[1,2,4]triazolo[1,5-a]pyridin-6-yl)-2-methylpyridin-3-yl)-3-(3,4-difluorophenyl)isoxazolidine-2-carboxamide: <56> (S)—N-(5-(2-amino-[1,2,4]triazolo[1,5-a]pyridin-6-yl)-2-methylpyridin-3-yl)-3-(4-fluorophenyl)isoxazolidine-2-carboxamide: <57> (S)—N-(5-(2-amino-[1,2,4]triazolo[1,5-a]pyridin-6-yl)-2-methoxypyridin-3-yl)-3-(3,5-difluorophenyl)isoxazolidine-2-carboxamide: <58> (S)—N-(5-(2-amino-[1,2,4]triazolo[1,5-a]pyridin-6-yl)-2-methoxypyridin-3-yl)-3-(3-fluorophenyl)isoxazolidine-2-carboxamide: <59> (S)—N-(5-(2-amino-[1,2,4]triazolo[1,5-a]pyridin-6-yl)-2-methylphenyl)-3-(3,5-difluorophenyl)isoxazolidine-2-carboxamide: <60> (S)—N-(5-(2-amino-[1,2,4]triazolo[1,5-a]pyridin-6-yl)-2-methylphenyl)-3-(3-fluorophenyl)isoxazolidine-2-carboxamide: <61> (S)—N-(5-(2-amino-[1,2,4]triazolo[1,5-a]pyridin-7-yl)-2-methylphenyl)-3-phenylisoxazolidine-2-carboxamide: <62> (S)—N-(5-(2-amino-[1,2,4]triazolo[1,5-a]pyridin-7-yl)-2-methylphenyl)-3-(3-fluorophenyl)isoxazolidine-2-carboxamide: <63> (S)—N-(5-(2-amino-[1,2,4]triazolo[1,5-a]pyridin-7-yl)-2-methylphenyl)-3-(3,5-difluorophenyl)isoxazolidine-2-carboxamide; <64> (S)—N-(5-(2-amino-[1,2,4]triazolo[1,5-a]pyridin-7-yl)-2-methylphenyl)-3-(4-fluorophenyl)isoxazolidine-2-carboxamide: <65> (S)—N-(5-(2-amino-[1,2,4]triazolo[1,5-a]pyridin-7-yl)-2-methylphenyl)-3-(4-chlorophenyl)isoxazolidine-2-carboxamide: <66> (S)—N-(5-(2-amino-[1,2,4]triazolo[1,5-a]pyridin-6-yl)-2-methylphenyl)-3-(4-fluorophenyl)isoxazolidine-2-carboxamide: <67> (S)—N-(5-(2-amino-[1,2,4]triazolo[1,5-a]pyridin-6-yl)-2-methylphenyl)-3-(4-chlorophenyl)isoxazolidine-2-carboxamide: <68> (S)—N-(5-(2-amino-[1,2,4]triazolo[1,5-a]pyridin-6-yl)-2-methylpyridin-3-yl)-3-(4-cyanophenyl)isoxazolidine-2-carboxamide; <69> (S)—N-(5-(2-amino-[1,2,4]triazolo[1,5-a]pyridin-6-yl)-2-methylpyridin-3-yl)-3-(4-chlorophenyl)isoxazolidine-2-carboxamide: <70> (S)—N-(5-(2-amino-[1,2,4]triazolo[1,5-a]pyridin-7-yl)-2-methylpyridin-3-yl)-3-(4-cyanophenyl)isoxazolidine-2-carboxamide: <71> (S)—N-(5-(2-amino-[1,2,4]triazolo[1,5-a]pyridin-6-yl)-2-methylpyridin-3-yl)-3-(naphthalen-2-yl) isoxazolidine-2-carboxamide: <72> (S)—N-(5-(2-amino-[1,2,4]triazolo[1,5-a]pyridin-6-yl)-2-methylpyridin-3-yl)-3-(3-(dimethylamino)phenyl)isoxazolidine-2-carboxamide; <73> (S)—N-(5-(2-amino-[1,2,4]triazolo[1,5-a]pyridin-6-yl)-2-methylpyridin-3-yl)-3-(3-chloro-2-methylphenyl)isoxazolidine-2-carboxamide: <74> (S)—N-(5-(2-amino-[1,2,4]triazolo[1,5-a]pyridin-6-yl)-2-methylpyridin-3-yl)-3-(4-methoxyphenyl)isoxazolidine-2-carboxamide: <75> (S)—N-(5-(2-amino-[1,2,4]triazolo[1,5-a]pyridin-6-yl)-2-methylpyridin-3-yl)-3-(4-(trifluoromethyl) thiazol-2-yl) isoxazolidine-2-carboxamide: <76> (S)—N-(5-(2-amino-[1,2,4]triazolo[1,5-a]pyridin-6-yl)-2-methylpyridin-3-yl)-3-(1-methyl-1H-pyrazol-4-yl) isoxazolidine-2-carboxamide: <77> (S)—N-(5-(2-amino-[1,2,4]triazolo[1,5-a]pyridin-6-yl)-2-methylpyridin-3-yl)-3-(furan-2-yl) isoxazolidine-2-carboxamide: <78> (S)—N-(5-(2-amino-[1,2,4]triazolo[1,5-a]pyridin-6-yl)-2-methylpyridin-3-yl)-3-(5-fluoropyridin-3-yl) isoxazolidine-2-carboxamide; <79> (R)—N-(5-(2-amino-[1,2,4]triazolo[1,5-a]pyridin-6-yl)-2-methylpyridin-3-yl)-3-(5-fluoropyridin-3-yl) isoxazolidine-2-carboxamide; <80> (S)—N-(5-(2-(cyclopropanecarboxamido)imidazo[1,2-b]pyridazin-6-yl)-2-methylphenyl)-3-phenylisoxazolidine-2-carboxamide: <81> (S)—N-(5-(2-acetamidoimidazo[1,2-b]pyridazin-6-yl)-2-methylphenyl)-3-phenylisoxazolidine-2-carboxamide; <82> (S)—N-(5-(2-(cyclopropanecarboxamido)imidazo[1,2-b]pyridazin-6-yl)-2-methylphenyl)-3-(4-fluorophenyl)isoxazolidine-2-carboxamide: <83> (S)—N-(5-(2-(cyclopropanecarboxamido) thiazolo[5,4-b]pyridin-5-yl)-2-methylphenyl)-3-(4-fluorophenyl)isoxazolidine-2-carboxamide; <84> (S)—N-(5-(2-((1R,2R)-2-fluorocyclopropane-1-carboxamido)benzo[d]thiazol-6-yl)-2-methylphenyl)-3-phenylisoxazolidine-2-carboxamide: <85> (S)—N-(5-(2-((1S,2S)-2-fluorocyclopropane-1-carboxamido)benzo[d]thiazol-6-yl)-2-methylphenyl)-3-phenylisoxazolidine-2-carboxamide: <86> (S)—N-(5-(2-((1R,2R)-2-fluorocyclopropane-1-carboxamido)imidazo[1,2-b]pyridazin-6-yl)-2-methylphenyl)-3-phenylisoxazolidine-2-carboxamide: <87> (S)—N-(5-(2-((1S,2S)-2-fluorocyclopropane-1-carboxamido)imidazo[1,2-b]pyridazin-6-yl)-2-methylphenyl)-3-phenylisoxazolidine-2-carboxamide: <88> (S)—N-(5-(2-((1S,2S)-2-fluorocyclopropane-1-carboxamido) thiazolo[5,4-b]pyridin-5-yl)-2-methylphenyl)-3-phenylisoxazolidine-2-carboxamide; <89> (S)—N-(5-(2-aminobenzo[d]thiazol-6-yl)-2-methylphenyl)-3-phenylisoxazolidine-2-carboxamide: <90> (S)—N-(5-(2-aminothiazolo[5,4-b]pyridin-5-yl)-2-methylphenyl)-3-(4-fluorophenyl)isoxazolidine-2-carboxamide; <91> (S)—N-(5-(2-amino-[1,2,4]triazolo[1,5-a]pyridin-6-yl)-2-methylphenyl)-3-(4-cyanophenyl)isoxazolidine-2-carboxamide: <92> (S)—N-(5-(2-amino-[1,2,4]triazolo[1,5-a]pyridin-7-yl)-2-methylphenyl)-3-(4-cyanophenyl)isoxazolidine-2-carboxamide: <93> (S)-3-(4-cyanophenyl)-N-(5-(2-(cyclopropanecarboxamido)benzo[d]thiazol-6-yl)-2-methylphenyl)isoxazolidine-2-carboxamide: <94> (S)-3-(4-cyanophenyl)-N-(5-(2-(cyclopropanecarboxamido) thiazolo[5,4-b]pyridin-5-yl)-2-methylphenyl)isoxazolidine-2-carboxamide: <95> (S)—N-(5-(2-aminoimidazo[1,2-b]pyridazin-6-yl)-2-methylphenyl)-3-phenylisoxazolidine-2-carboxamide: <96> (S)—N-(5-(2-amino-[1,2,4]triazolo[1,5-a]pyridin-6-yl)-2-fluorophenyl)-3-phenylisoxazolidine-2-carboxamide: <97> (S)—N-(5-(2-amino-[1,2,4]triazolo[1,5-a]pyridin-6-yl)-2-chlorophenyl)-3-phenylisoxazolidine-2-carboxamide; <98> (S)—N-(5-(2-aminothiazolo[5,4-b]pyridin-5-yl)-2-methylphenyl)-3-phenylisoxazolidine-2-carboxamide: <99> (S)—N-(5-(4-aminopyrrolo[2,1-f][1,2,4]triazin-7-yl)-2-methoxypyridin-3-yl)-3-phenylisoxazolidine-2-carboxamide: <100> (S)—N-(5-(4-aminopyrrolo[2,1-f][1,2,4]triazin-7-yl)-2-methylphenyl)-3-phenylisoxazolidine-2-carboxamide: <101> (S)—N-(5-([1,2,4]triazolo[1,5-a]pyridin-6-yl)-2-methylphenyl)-3-phenylisoxazolidine-2-carboxamide: <102> (S)—N-(5-([1,2,4]triazolo[1,5-a]pyridin-7-yl)-2-methylphenyl)-3-phenylisoxazolidine-2-carboxamide: <103> (S)—N-(3-(2-amino-[1,2,4]triazolo[1,5-a]pyridin-7-yl)-2-fluoro-6-methoxyphenyl)-3-phenylisoxazolidine-2-carboxamide: <104> (S)—N-(3-(2-amino-[1,2,4]triazolo[1,5-a]pyridin-6-yl)-2-fluoro-6-methoxyphenyl)-3-phenylisoxazolidine-2-carboxamide; <105> (S)—N-(3-(2-amino-[1,2,4]triazolo[1,5-a]pyridin-6-yl)-6-chloro-2-fluorophenyl)-3-phenylisoxazolidine-2-carboxamide: <106> (S)—N-(5-([1,2,4]triazolo[4,3-b]pyridazin-6-yl)-2-methylphenyl)-3-phenylisoxazolidine-2-carboxamide: <107> (S)—N-(2-methyl-5-(3-methyl-[1,2,4]triazolo[4,3-b]pyridazin-6-yl)phenyl)-3-phenylisoxazolidine-2-carboxamide; <108> (S)—N-(2-methyl-5-(3-(trifluoromethyl)-[1,2,4]triazolo[4,3-b]pyridazin-6-yl)phenyl)-3-phenylisoxazolidine-2-carboxamide: <109> (S)—N-(5-(2-aminobenzo[d]oxazol-6-yl)-2-methylphenyl)-3-phenylisoxazolidine-2-carboxamide; <110> (S)—N-(5-(2-aminobenzo[d]oxazol-6-yl)-2-methoxypyridin-3-yl)-3-phenylisoxazolidine-2-carboxamide; <111> (S)—N-(5-(2-aminoimidazo[1,2-a]pyridin-6-yl)-2-methoxypyridin-3-yl)-3-phenylisoxazolidine-2-carboxamide: <112> (S)—N-(5-(imidazo[1,2-b]pyridazin-6-yl)-2-methylphenyl)-3-phenylisoxazolidine-2-carboxamide: <113> (S)—N-(5-(imidazo[1,2-a]pyridin-6-yl)-2-methylphenyl)-3-phenylisoxazolidine-2-carboxamide: <114> (S)—N-(5-(3-cyanoimidazo[1,2-a]pyridin-6-yl)-2-methylphenyl)-3-phenylisoxazolidine-2-carboxamide: <115> (S)—N-(5-(3-(3-hydroxy-3-methylbut-1-yn-1-yl)imidazo[1,2-a]pyridin-6-yl)-2-methylphenyl)-3-phenylisoxazolidine-2-carboxamide: <116> (S)—N-(5-(3-ethynylimidazo[1,2-a]pyridin-6-yl)-2-methylphenyl)-3-phenylisoxazolidine-2-carboxamide; <117> (S)—N-(5-(3-(3-hydroxy-3-methylbut-1-yn-1-yl)imidazo[1,2-b]pyridazin-6-yl)-2-methylphenyl)-3-6-yl)-2-methylphenyl)-3-phenylisoxazolidine-2-carboxamide; <119> (S)—N-(3-(4-aminopyrrolo[2,1-f][1,2,4]triazin-7-yl)phenyl)-3-phenylisoxazolidine-2-carboxamide; <120> (S)—N-(5-(4-aminopyrrolo[2,1-f][1,2,4]triazin-7-yl)-2-methylpyridin-3-yl)-3-phenylisoxazolidine-2-carboxamide; <121> (S)—N-(5-(4-aminothieno[3,2-d]pyrimidin-7-yl)-2-methylpyridin-3-yl)-3-phenylisoxazolidine-2-carboxamide; <122> (S)—N-(5-(4-aminothieno[3,2-d]pyrimidin-7-yl)-2-methylphenyl)-3-phenylisoxazolidine-2-carboxamide: <123> (S)—N-(5-(2-amino-[1,2,4]triazolo[1,5-a]pyridin-7-yl)-2-fluorophenyl)-3-phenylisoxazolidine-2-carboxamide: <124> (S)—N-(5-(2-amino-[1,2,4]triazolo[1,5-a]pyridin-7-yl)-2-chlorophenyl)-3-phenylisoxazolidine-2-carboxamide: <125> (S)—N-(5-(3-ethynylimidazo[1,2-b]pyridazin-6-yl)-2-methylphenyl)-3-phenylisoxazolidine-2-carboxamide: <126> (S)—N-(5-(2-aminopyrazolo[1,5-a]pyridin-5-yl)-2-methylphenyl)-3-phenylisoxazolidine-2-carboxamide: <127> (S)—N-(3-(4-aminopyrrolo[2,1-f][1,2,4]triazin-7-yl)-4-methylphenyl)-3-phenylisoxazolidine-2-carboxamide: <128> (S)—N-(5-(4-aminothieno[3,2-d]pyrimidin-7-yl)-2-methoxypyridin-3-yl)-3-phenylisoxazolidine-2-carboxamide: <129> (S)—N-(5-(4-amino-5-(trifluoromethyl)pyrrolo[2,1-f][1,2,4]triazin-7-yl)-2-methoxypyridin-3-yl)-3-phenylisoxazolidine-2-carboxamide; <130> (S)—N-(2-methyl-5-(thiazolo[4,5-c]pyridin-6-yl)phenyl)-3-phenylisoxazolidine-2-carboxamide; <131> (S)—N-(2-methyl-5-(thiazolo[5,4-b]pyridin-5-yl)phenyl)-3-phenylisoxazolidine-2-carboxamide: <132> (S)—N-(3-(2-aminobenzo[d]thiazol-6-yl)-2,6-difluorophenyl)-3-phenylisoxazolidine-2-carboxamide: <133> (S)—N-(2-methyl-5-(1-methyl-1H-imidazo[4,5-c]pyridin-6-yl)phenyl)-3-phenylisoxazolidine-2-carboxamide: <134> (S)—N-(3-(2-amino-[1,2,4]triazolo[1,5-a]pyridin-7-yl)-2,6-difluorophenyl)-3-phenylisoxazolidine-2-carboxamide: <135> (S)—N-(5-(2-amino-3-((1S,2S)-2-fluorocyclopropane-1-carbonyl)imidazo[1,2-a]pyridin-6-yl)-2-methylphenyl)-3-5-yl)-2-methylphenyl)-3-phenylmorpholine-4-carboxamide: <137> (S)—N-(5-(2-aminopyrazolo[1,5-a]pyridin-5-yl)-2-methylphenyl)-2-phenylmorpholine-4-carboxamide: <138> (S)—N-(5-(2-aminopyrazolo[1,5-a]pyridin-5-yl)-2-methylphenyl)-3-phenylbenzo[d]isooxazole-2 (3H)-carboxamide; <139> (R)—N-(5-(2-aminopyrazolo[1,5-a]pyridin-5-yl)-2-methylphenyl)-3-phenylbenzo[d]isoxazole-2 (3H)-carboxamide; <140> (R)—N-(5-(2-aminopyrazolo[1,5-a]pyridin-5-yl)-2-methylphenyl)-7-phenyl-5-oxa-6-azaspiro[2.4]heptane-6-carboxamide; <141> (S)—N-(5-(2-aminopyrazolo[1,5-a]pyridin-5-yl)-2-methylphenyl)-3-methyl-3-phenylisoxazolidine-2-carboxamide: <142> (R)—N-(5-(2-aminopyrazolo[1,5-a]pyridin-5-yl)-2-methylphenyl)-3-methyl-3-phenylisoxazolidine-2-carboxamide: <143> (S)—N-(2-methyl-5-(3-methyl-3H-imidazo[4,5-b]pyridin-5-yl)phenyl)-3-phenylisoxazolidine-2-carboxamide; <144> (S)—N-(5-(2-amino-[1,2,4]triazolo[1,5-a]pyridin-6-yl)-2-cyanophenyl)-3-phenylisoxazolidine-2-carboxamide: <145> (S)—N-(5-(2-amino-[1,2,4]triazolo[1,5-a]pyridin-6-yl)-2-methylphenyl)-3-phenyl-1,2-oxazinane-2-carboxamide: <146> (S)—N-(5-(2-amino-[1,2,4]triazolo[1,5-a]pyridin-6-yl)-2-methylphenyl)-3-(4-fluorophenyl)-1,2-oxazinane-2-carboxamide; <147> (S)—N-(5-(2-amino-[1,2,4]triazolo[1,5-a]pyridin-6-yl)-2-methylphenyl)-3-(3,5-difluorophenyl)-1,2-oxazinane-2-carboxamide: <148> (S)—N-(5-(2-amino-[1,2,4]triazolo[1,5-a]pyridin-7-yl)-2-methylphenyl)-3-phenyl-1,2-oxazinane-2-carboxamide: <149> (S)—N-(3-(2-amino-[1,2,4]triazolo[1,5-a]pyridin-6-yl)-2-fluoro-6-methylphenyl)-3-phenylisoxazolidine-2-carboxamide: <150> (S)—N-(3-(2-amino-[1,2,4]triazolo[1,5-a]pyridin-6-yl)-6-ethyl-2-fluorophenyl)-3-phenylisoxazolidine-2-carboxamide; <151> (S)—N-(3-(2-amino-[1,2,4]triazolo[1,5-a]pyridin-6-yl)-6-cyclopropyl-2-fluorophenyl)-3-phenylisoxazolidine-2-carboxamide: <152> (S)—N-(5-(2-amino-[1,2,4]triazolo[1,5-a]pyridin-6-yl)-2-ethylphenyl)-3-phenylisoxazolidine-2-carboxamide: <153> (S)—N-(5-(2-amino-[1,2,4]triazolo[1,5-a]pyridin-6-yl)-2-cyclopropylphenyl)-3-phenylisoxazolidine-2-carboxamide; <154> (S)—N-(3-(2-amino-[1,2,4]triazolo[1,5-a]pyridin-7-yl)-2-fluoro-6-methylphenyl)-3-phenylisoxazolidine-2-carboxamide; <155> (S)—N-(3-(2-amino-[1,2,4]triazolo[1,5-a]pyridin-7-yl)-6-ethyl-2-fluorophenyl)-3-phenylisoxazolidine-2-carboxamide; <156> (S)—N-(3-(2-amino-[1,2,4]triazolo[1,5-a]pyridin-7-yl)-6-cyclopropyl-2-fluorophenyl)-3-phenylisoxazolidine-2-carboxamide; <157> (S)—N-(5-(2-amino-[1,2,4]triazolo[1,5-a]pyridin-7-yl)-2-ethylphenyl)-3-phenylisoxazolidine-2-carboxamide; and <158> (S)—N-(5-(2-amino-[1,2,4]triazolo[1,5-a]pyridin-7-yl)-2-cyclopropylphenyl)-3-phenylisoxazolidine-2-carboxamide.

7. A method of preparing a compound of Chemical Formula 1, comprising:
preparing a compound of Chemical Formula 3 from a compound of Chemical Formula 2 (Step 1); and
preparing a compound of Chemical Formula 1 from the compound of Chemical Formula 3 (Step 2), 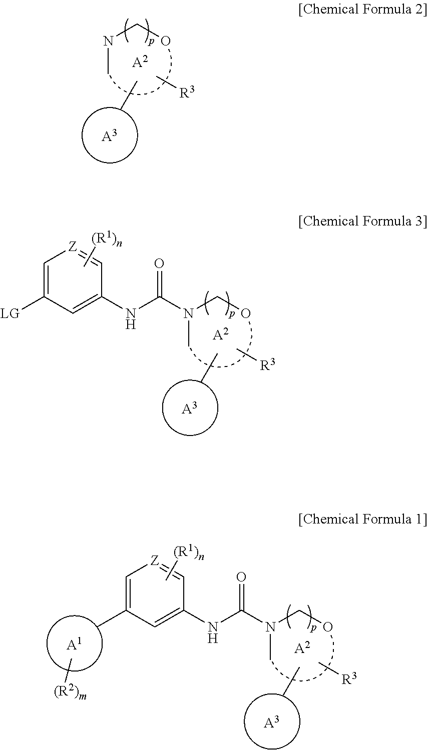
In the above formulas, LG is a leaving group, and Z, rings A¹ to A³, R¹ to R³, m, n, and p are the same as defined in claim 1.

8. A pharmaceutical composition comprising the compound of Chemical Formula 1 according to claim 1, an isomer thereof, a solvate thereof, a hydrate thereof, or a pharmaceutically acceptable salt thereof as an active ingredient, for preventing or treating a disease selected from the group consisting of inflammatory bowel disease, Crohn's disease, ulcerative colitis, psoriasis, retinal detachment, retinitis pigmentosa, macular degeneration, pancreatitis, atopic dermatitis, rheumatoid arthritis, spondyloarthritis, gout, Sjogren's syndrome, systemic scleroderma, anti-phospholipid syndrome, vasculitis, osteoarthritis, non-alcoholic steatohepatitis, alcoholic steatohepatitis, primary sclerosing cholangitis, nephritis, celiac disease, transplant rejection, sepsis, systemic inflammatory response syndrome, myocardial infarction, Huntington's disease, Alzheimer's disease, Parkinson's disease, allergic disease, asthma, atopic dermatitis, multiple sclerosis, type I diabetes, Wegener's granulomatosis, pulmonary sarcoidosis, Behcet's disease, motor neurone disease, chronic obstructive pulmonary disease, and periodontitis.

9. The pharmaceutical composition of claim 8, which inhibits the expression or activity of RIPK1 kinase.

10. A method of preventing or treating an RIPK1 kinase-related disease, comprising:
administering the compound of Chemical Formula 1 according to claim 1, an isomer thereof, a solvate thereof, a hydrate thereof, or a pharmaceutically acceptable salt thereof to a subject in need thereof at a pharmaceutically effective amount.

11. The method of claim 10, wherein the RIPK1 kinase-related disease is selected from the group consisting of inflammatory bowel disease, Crohn's disease, ulcerative colitis, psoriasis, retinal detachment, retinitis pigmentosa, macular degeneration, pancreatitis, atopic dermatitis, rheumatoid arthritis, spondyloarthritis, gout, Sjogren's syndrome, systemic scleroderma, anti-phospholipid syndrome, vasculitis, osteoarthritis, non-alcoholic steatohepatitis, alcoholic steatohepatitis, primary sclerosing cholangitis, nephritis, celiac disease, transplant rejection, sepsis, systemic inflammatory response syndrome, myocardial infarction, Huntington's disease, Alzheimer's disease, Parkinson's disease, allergic disease, asthma, atopic dermatitis, multiple sclerosis, type I diabetes, Wegener's granulomatosis, pulmonary sarcoidosis, Behcet's disease, motor neurone disease, chronic obstructive pulmonary disease, and periodontitis.

12. (canceled)

13. (canceled)
